(19) United States
(12) Patent Application Publication
(10) Pub. No.: US 2019/0295713 A1
(43) Pub. Date: Sep. 26, 2019

(54) Health Care Information Management Platform

(71) Applicant: MDout Inc., Livermore, CA (US)

(72) Inventors: Michael Sarwari, Livermore, CA (US); Arch Phang, Livermore, CA (US)

(73) Assignee: MDout Inc., Livermore, CA (US)

(21) Appl. No.: 16/361,001

(22) Filed: Mar. 21, 2019

Related U.S. Application Data
(60) Provisional application No. 62/646,117, filed on Mar. 21, 2018.

(51) Int. Cl. G16H 40/20; G16H 10/60

(52) U.S. Cl. CPC G16H 40/20; G16H 10/60

(57) ABSTRACT

A web-based health care information management system is provided, comprising at least one computer server connected to a network. The server comprises a processor and a memory unit accessible to the processor. The processor is configured to provide an interactive website accessible to at least one patient of at least one clinic, at least one staff member of at least one clinic, and at least one administrator of at least one clinic, and at least one system administrator.

### CROSS-REFERENCES TO RELATED APPLICATIONS

The present application claims priority from U.S. Provisional Application Ser. No. 62/646,117 filed on Mar. 21, 2018, which is hereby incorporated herein by reference in its entirety.

### FIELD OF INVENTION

The present invention relates to the field of information systems for the health industry.

### BACKGROUND

On Oct. 6, 2015 an article in Money Magazine written by Brad Tuttle indicated that research was published in the American Journal of Managed Care estimating that on average it took 124 minutes each time a person seeks medical care. The total time included about 37 minutes of travel time, and approximately 87 minutes at the doctor's office or clinic. Of that time on average only 20 minutes was spent with the doctor. The rest of the time was spent doing paperwork and waiting for the doctor.

The Opportunity: According to Advertising Age, a recent healthcare marketing report indicates that in 2016 the advertising spending for the U.S. healthcare industry hit a record $9.7 billion which was up 11 percent from the previous year. The report also indicates that consumers are increasingly looking online for answers to health-related questions.

### BRIEF SUMMARY OF THE INVENTION

There is therefore a need to improve the patients' experience at clinics by speeding up form filling and educating the patients about health-related topics relevant to the patients, while the patients wait for the doctor or at any time before and after their doctor's appointments.

The platform of the present invention also enables clinics to post content relevant to their patients, thereby educating their patients about health-related topics. In some embodiments the present invention, the platform helps medical and pharmaceutical companies reach specific targeted consumers with ads about products relevant to the patients.

Therefore, an aspect of some embodiments of the present invention relates to a web-based health care information management system comprising at least one computer server connected to a network, the server comprising a processor and a memory unit accessible to the processor. The processor is configured to generate an interactive website accessible to at least one patient of at least one clinic, at least one staff member of at least one clinic, and at least one administrator of at least one clinic, and at least one system administrator. The processor is configured to provide via the interactive web site a system management tool to each system administrator, the processor being configured to: generate one or more clinic administrator profiles in a first database of clinic administrators, in response to an input from the system administrator; receive from the system administrator informative videos and articles in a second database of content and receive a tag for each video and article with a category selected from a predetermined list of categories; receive from the system administrator advertisement videos in a third database of advertisements and receive a tag for each advertisements video with a category selected from the list of categories. The processor is configured to provide via the interactive website a clinic management tool to each clinic administrator, the processor being configured to: generate one or more profiles of medical staff associated with the clinic to a fourth database of staff members, in response to an input from the clinic administrator; generate one or more profiles of patients associated with the clinic in a fifth database of patients, in response to an input from the clinic administrator; receive from the clinic administrator one or more informative videos and articles in the second database and a tag for each video and article with a category selected from the list of categories; generate questionnaires from scratch or from predetermined questionnaire templates in response to an input from the clinic's administrator, and add the questionnaire to a sixth database of questionnaires; assign one or more patients to one or more staff members of the clinic in response to an input from the clinic's administrator; assign one or more of the questionnaires to one or more patients in response to an input from the clinic's administrator; display to the clinic administrator a screen indicative of a completion status of the assigned questionnaires. The processor is configured to provide via the interactive web site a staff management tool to each staff member, the processor being configured to: display a list of one or more patients assigned to the staff member; display a list of surveys assigned to the one or more patients assigned to the staff member; receive from the staff member notes associated with any survey assigned to a patient assigned to the staff member; display notes previously added to any survey assigned to a patient assigned to the staff member. The processor is configured to provide via the interactive website a patient management tool to each patient, the processor being configured to: display to the patient a list of surveys assigned to the patient; receive from the patient answers to one or more of the surveys assigned to the patient; display one or more informative videos and/or articles from the second database, such that, when the patient views a video or reads an article tagged with a category from the list of categories, at least one advertisement tagged with a category which is the same as the category of the video viewed by the patient or the article read by the user is shown to the user.

In a variant, the processor is configured, via the system management tool, to generate one or more of the questionnaire templates and add the templates to the sixth database of questionnaires, in response to an input from the system administrator.

In another variant, each video and each article added to the system via the clinic management tool is designated as public or private. A private video is a video viewable by medical staff and patients associated with the clinic. A public video is a video viewable by all users of the server, pending review by a system administrator. A private article is an article viewable by medical staff and patients associated with the clinic. A public article is a video viewable by all users of the server, pending review by a system administrator.

In yet another variant, when a new private video or a new private article is received in the second database by a clinic's administrator, the server is configured to send a message to each patient associated with the clinic's administrator's clinic to inform each patient that the new private video or the new private article has been added.

In a further variant, when a questionnaire is assigned to a patient by a clinic administrator, the server is configured to send a message to the patient to inform the patient that the questionnaire has been assigned to the patient.

Another aspect of some embodiments of the present invention relates to a web-based health care information management system comprising at least one computer server connected to a network. The server includes a processor and a memory unit accessible to the processor. The processor is configured to provide an interactive website accessible to at least one patient of at least one clinic, at least one staff member of at least one clinic, and at least one administrator of at least one clinic, and at least one system administrator. The processor is configured to provide via the interactive website a system management tool to each system administrator, to enable the system administrator to: add one or more clinic administrator profiles to a first database of clinic administrators; add informative videos and articles to a second database of content and tag each videos and articles with a category selected from a predetermined list of categories; add advertisement videos to a third database of advertisements and tag the advertisements with a category selected from the list of categories. The processor is configured to provide via the interactive website a clinic management tool to each clinic administrator, to enable the clinic administrator to: add one or more profiles of medical staff associated with the clinic to a fourth database of staff members; add one or more profiles of patients associated with the clinic to a fifth database of patients; add one or more informative videos and articles to the second database and tag each videos and articles with a category selected from the list of categories; compose questionnaires from scratch or from predetermined questionnaire templates, and add the questionnaire to a sixth database of questionnaires; assign one or more patients to one or more staff members of the clinic; assign one or more of the questionnaires to one or more patients; monitor completion of the assigned questionnaires by the patients. The processor is configured to provide via the interactive web site a staff management tool to each staff member, to enable the staff member to: view a list of one or more patients assigned to the staff member; view a list of surveys assigned to the one or more patients assigned to the staff member; add notes to any survey assigned to a patient assigned to the staff member, and view notes previously added to the survey. The processor is configured to provide via the interactive website a patient management tool to each patient, to enable the patient to: view a list of surveys assigned to the patient; fill one or more of the surveys assigned to the patient; view one or more informative videos and/or articles from the second database, such that, when the patient views a video or reads an article tagged with a category from the list of categories, at least one advertisement tagged with a category which is the same as the category of the video viewed by the patient or the article read by the user is shown to the user.

In a variant, the processor is configured, via the system management tool, to enable the system administrator to compose one or more of the questionnaire templates and add the templates to the sixth database of questionnaires.

In another variant, each video and each article added to the system via the clinic management tool is designated as public or private. A private video is a video viewable by medical staff and patients associated with the clinic A public video is a video viewable by all users of the server, pending review by a system administrator. A private article is an article viewable by medical staff and patients associated with the clinic. A public article is a video viewable by all users of the server, pending review by a system administrator.

In yet another variant, when a new private video or a new private article is added to the second database by a clinic's administrator, the server sends a message to each patient associated with the clinic's administrator's clinic to inform each patient that the new private video or the new private article has been added.

In a further variant, when a questionnaire is assigned to a patient by a clinic administrator, the server sends a message to the patient to inform the patient that the questionnaire has been assigned to the patient.

### BRIEF DESCRIPTION OF DRAWINGS

FIG. 1 is a block diagram illustrating a health information system, according to some embodiments of the present invention;

FIG. 2 is a block diagram illustrating data stored in the server of a health information system of the present invention;

FIG. 3 is a screenshot of images displayed by a communication device connected to the system of FIGS. 1 and 2, to enable a registered user to log in, according to some embodiments of the present invention;

FIGS. 4a and 4b are screenshots of images displayed by a communication device connected to the system of FIGS. 1 and 2, to enable a patient of a registered clinic to register in the system, according to some embodiments of the present invention;

FIG. 5 is a screenshot of images displayed by a communication device connected to the system of FIGS. 1 and 2, to enable an administrator of an unregistered clinic to register the clinic in the system, according to some embodiments of the present invention;

FIGS. 6-38 are screenshots of images displayed by a communication device connected to the system of FIGS. 1 and 2 running the clinic management tool, according to some embodiments of the present invention;

FIGS. 39-45 are screenshots of images displayed by a communication device connected to the system of FIGS. 1 and 2 running the staff management tool, according to some embodiments of the present invention;

FIGS. 46-51 are screenshots of images displayed by a communication device connected to the system of FIGS. 1 and 2 running the patient management tool, according to some embodiments of the present invention; and

FIGS. 52-70 are screenshots of images by a communication device connected to the system of FIGS. 1 and 2 running the system management tool, according to some embodiments of the present invention.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

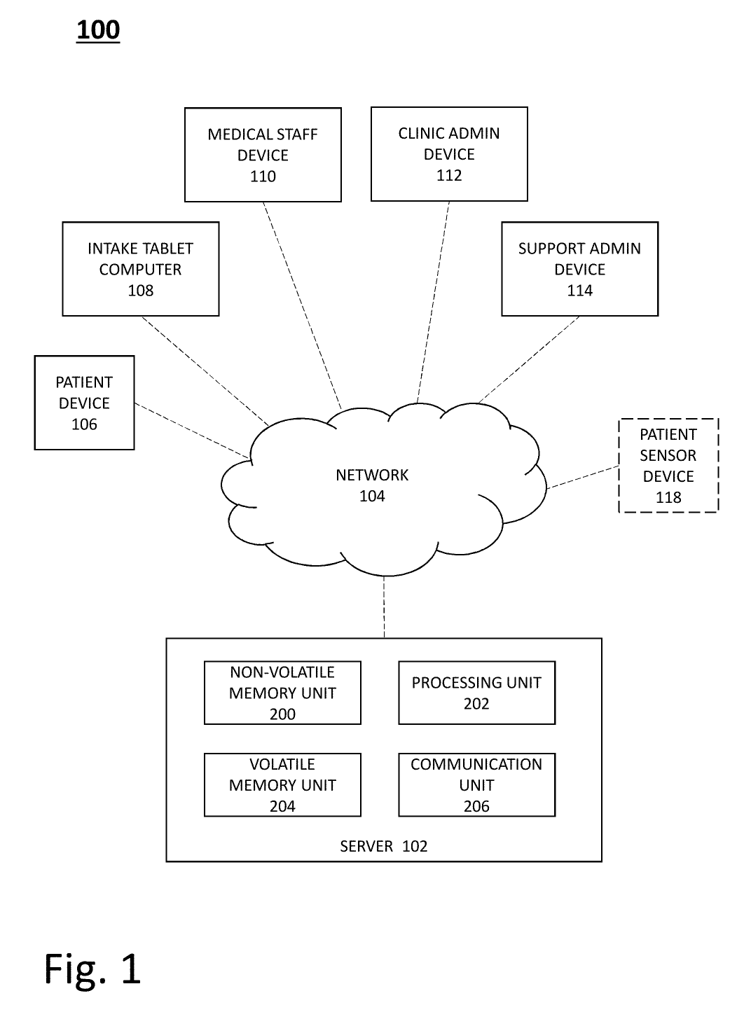

FIG. 1 is a block diagram illustrating a health information system, according to some embodiments of the present invention.

The information system 100 of the present invention includes a server 102 configured for communicating with a plurality of communication devices across a network 104. The network may be a private network or a public network, such as the internet. The server 102 includes a non-volatile memory unit 200, a processing unit 202, a volatile memory unit 204, and a communication unit 206. The units of the server 102 may be located at a single location or may be located remotely from each other while being in communication with each other.

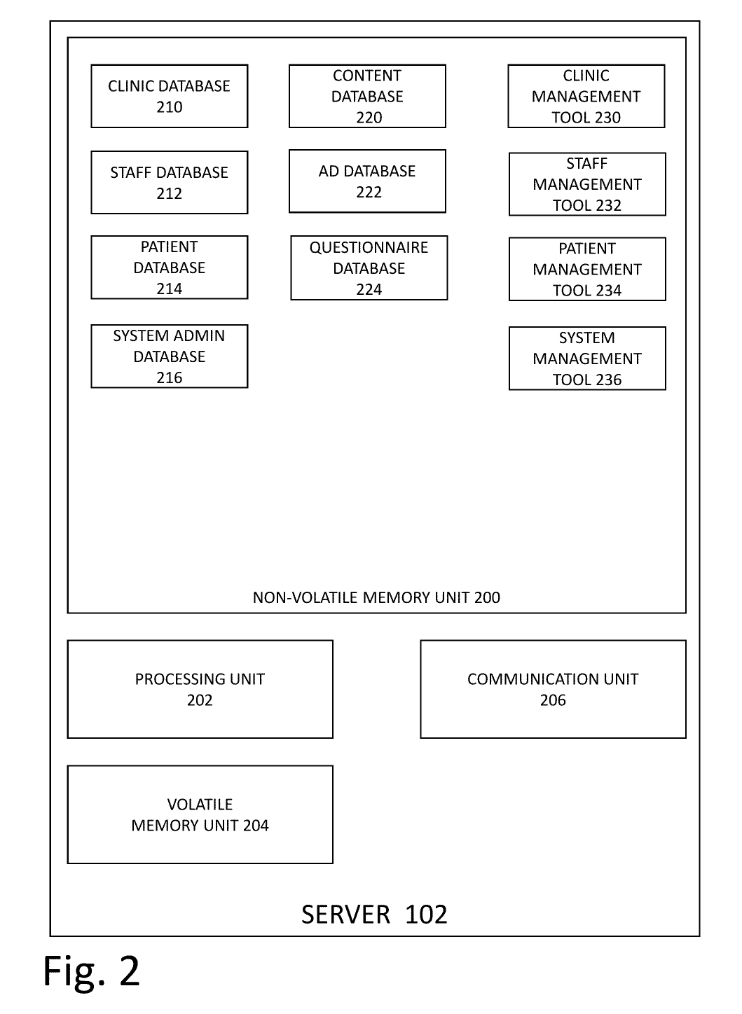

As will be shown in FIG. 2, the non-volatile memory unit 200 is configured for storing information configured for enabling interaction of users with the server, instructions for the operation of the server, and data uploaded by the users. The processing unit 202 is configured for processing and implementing the instructions and information stored in the non-volatile memory unit 200. The volatile memory unit 204 is configured for loading information, instructions, and data from the non-volatile memory unit 200 and feeding the information, instructions, and data to the processing unit 202 for fast processing. The communication unit 206 is configured for enabling communication between the server 102 and the communication devices via the network 104.

The communication devices are computing units in communication with the network 104. The communication devices may include smartphones, tablet computers, laptop computers, desktop computers, for example. Each user may access the server 102 via respective credentials (e.g., a username/email address and a password).

A patient may use a patient device 106 to access information relevant to the patient or assign to the patient. The patient may use an intake tablet computer 108 provided by the clinic to fill a form, read an article about a relevant health topic, and view a video about a relevant health clinic while waiting for a doctor's appointment at the clinic. Medical staff may use medical staff device 110 to access patient data, send content to the patients, create forms, and assign forms to patients. Medical staff can optionally complete after-visit reports about the patients and fill out prescription forms for their patients. A clinic administrator may use clinic administration device 112 to assign patients to medical staff members, add medical staff members to the clinic's database, add patients to the clinic's database, and add videos and articles to the clinic's database. A support administrator may use a support administrator device 114 to add videos and articles, view videos and articles added by clinic administrators (and optionally mark the videos and articles as suitable or unsuitable), add clinics to the system, manage advertisements.

It should be noted that the same device may be used by different types of the users to access the server. In fact once a request to access the server is made by any communication device, a login page is displayed to the user to prompt the user to enter the user's credentials. Once the user enters the credentials, the communication device communicates with the server and enables the user to do perform activities permitted to the user via the communication device.

In some embodiments of the present invention, sensor devices 118 associated with a patient may measure parameters associated with the patient's health (such as heartbeat, number of steps taken, body temperature etc.), and send the parameters to the server. The measured parameters may be accessible to the medical staff assigned to the patient, to enable the medical staff to better understand the patient's lifestyle and advise the patient accordingly. Sensor devices may include smart watches, and activity tracking devices.

FIG. 2 is a block diagram illustrating data stored in the server 102 of the health information system 100 of the present invention.

The non-volatile memory unit 200 is configured to store the following user databases: a clinic database 210, a staff database 212, a patient database 214, and a support administrator database 216. The non-volatile memory unit 200 is also configured to store a content database 220, an ad database 222, a questionnaire database 224. The non-volatile memory unit 200 is also configured to store instructions for the processing unit 202 to run: a clinic management tool 230, a staff management tool 232, a patient management tool 234, and a system management tool 236.

The clinic database 210 is a database of each clinic and credentials of clinic administrators assigned to the clinics. The staff database 212 is a database of all staff members of all clinics (except for clinic administrators) and credentials of staff members. Each staff member is assigned to one clinic (and optionally more than one clinic) in which the staff member works. The patient database 214 is a database of all patients and their credentials. Each patient is associated with one or more clinics at which the patient receives health care. Each patient is associated with one or more clinic staff members who provide health care to the patient. The support administrator database 216 is a database of all support administrators and their credentials.

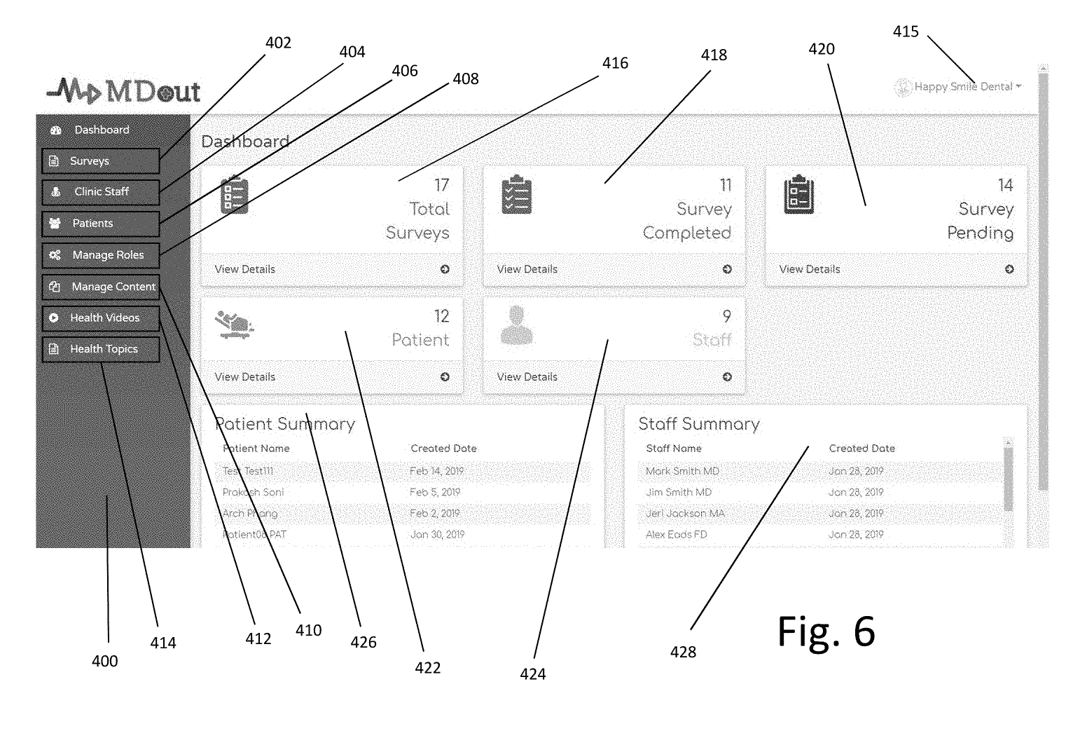

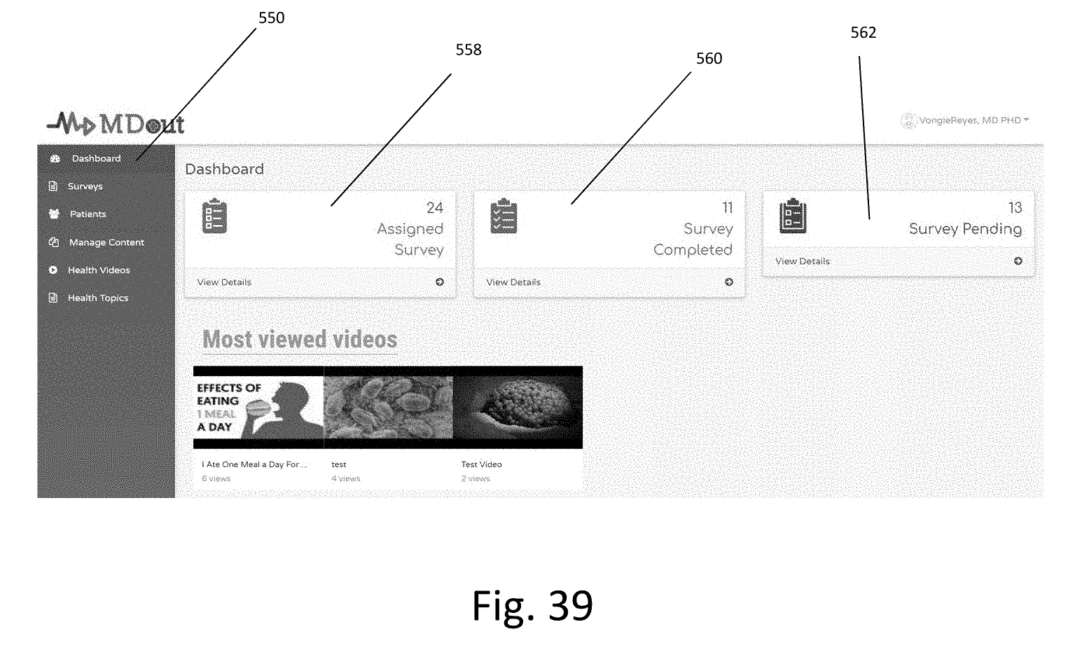

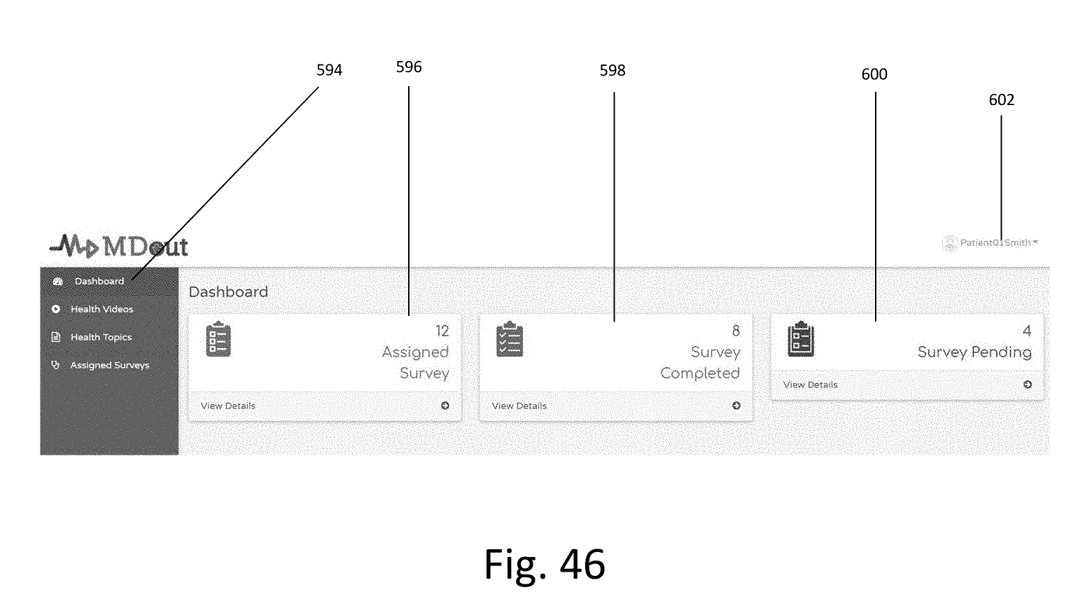

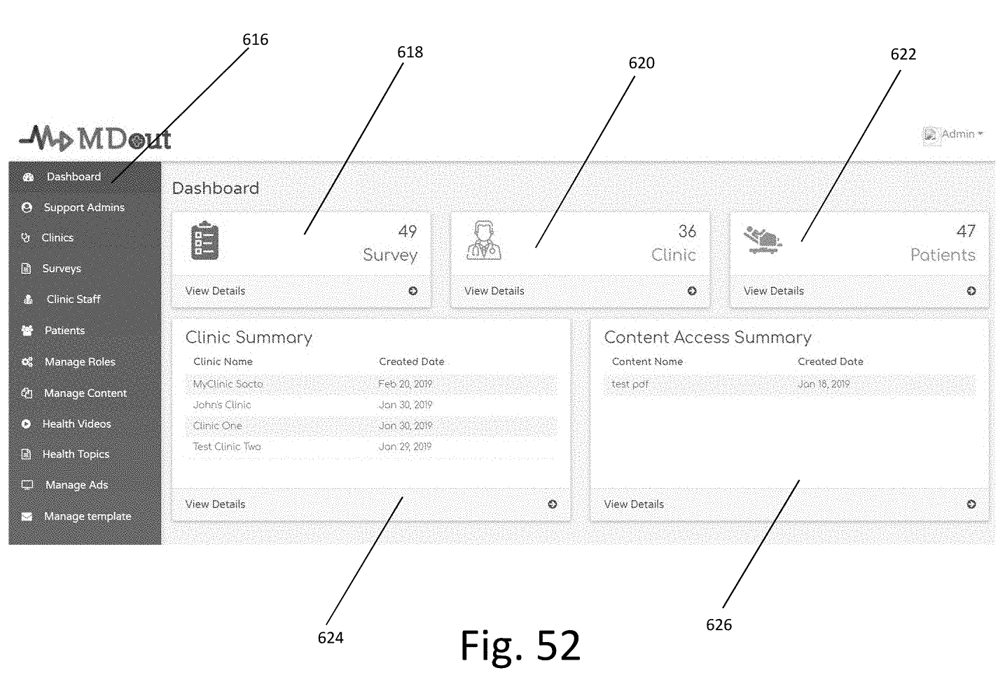

The processing unit 202 runs the clinic management tool 230 by causing a user's communication device to display images, text, links, and prompts which enable a clinic administrator to enter inputs to manage and view information relating the clinic, the clinic's medical staff, and the clinic's patients, as will be explained in FIGS. 6-38. The processing unit 202 runs the staff management tool 232 by causing a user's communication device to display images, text, links, and prompts which enable a medical staff member to enter inputs to manage and view information relating to the medical staff member and to patients assigned to the medical staff member, as will be explained in FIGS. 39-45. The processing unit 202 runs the patient management tool 234 by causing a user's communication device to display images, text, links, and prompts which enable a patient to enter inputs to view and manage information relating to the patient, as will be explained in FIGS. 46-51. The processing unit 202 runs the system management tool 236 by causing a communication device of a support administrator to display images, text, links, and prompts which enable an administrator of the system 100 to enter inputs to view and manage information relating to the system 100, as will be explained in FIGS. 52-70. The support administrator is an individual acting for the owner of the system for maintaining and managing the system 100. The support administrator is optionally privy to some information about clinics, medical staff, patients.

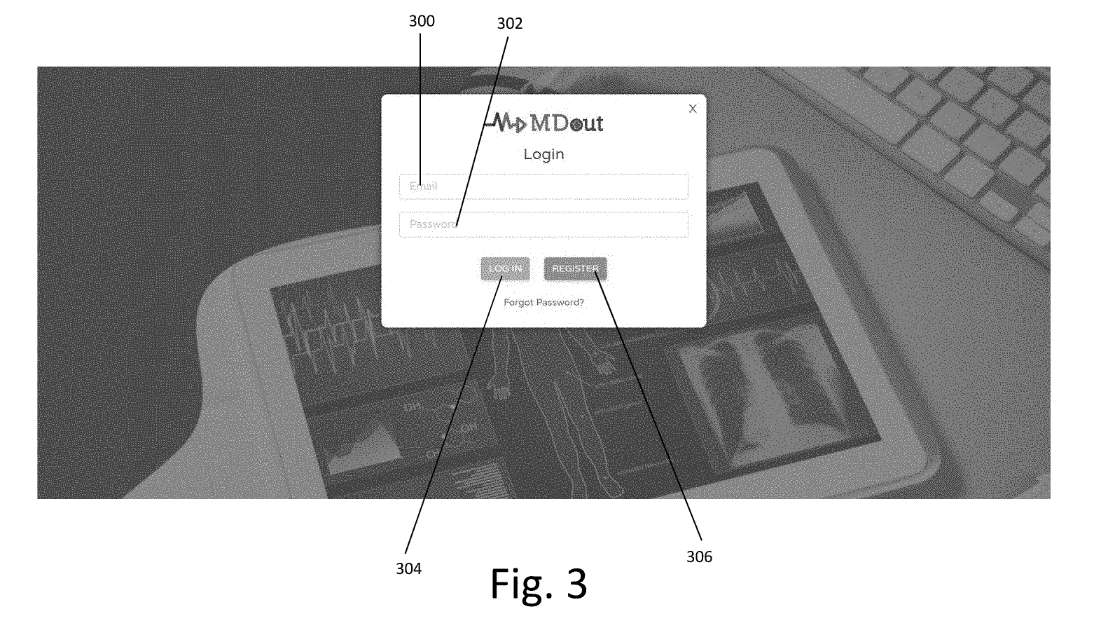

FIG. 3 is a screenshot of images displayed by a communication device connected to the system of FIGS. 1 and 2, to enable a registered user to log in, according to some embodiments of the present invention.

A user generally may request access to the server via a web portal. When a user (patient, medical staff member, clinic administrator, support administrator) wishes to access the server 102, the server causes the user's browser or application to display a page having a first text box 300, a second text box 302, a log-in button 304, and a register button 306.

If the user is a registered user, the user may type a first part of the user's credentials (e.g., email address, username, phone number, etc.) in the first text box 300, type a second part of the user's credentials (generally a password) in the second text box 302, and send the typed credentials to the server 102 by interacting with the log-in button 304. The interaction may with the log-in button (or any button or link that will be described hereafter) may be made, for example, by moving a pointer onto the button and clicking with a mouse or other pointing device, by touching an area of the screen with a user's finger or another object held by the user (if the user communication device has a touchscreen).

If the credentials are correct, the server 102 causes the user device to display an introduction page relevant to the user, as will be described below in FIGS. 6, 39, 46, and 52. Otherwise, the server 102 cause the user device to display a message informing the user that the credentials are not correct and that access to the server 102 has been denied.

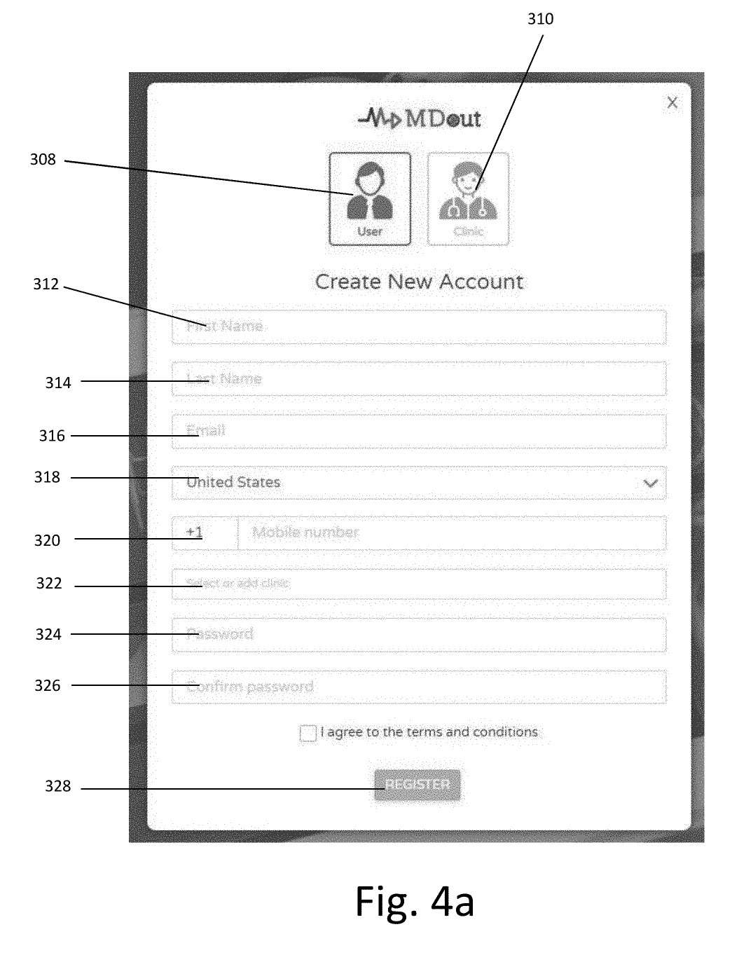

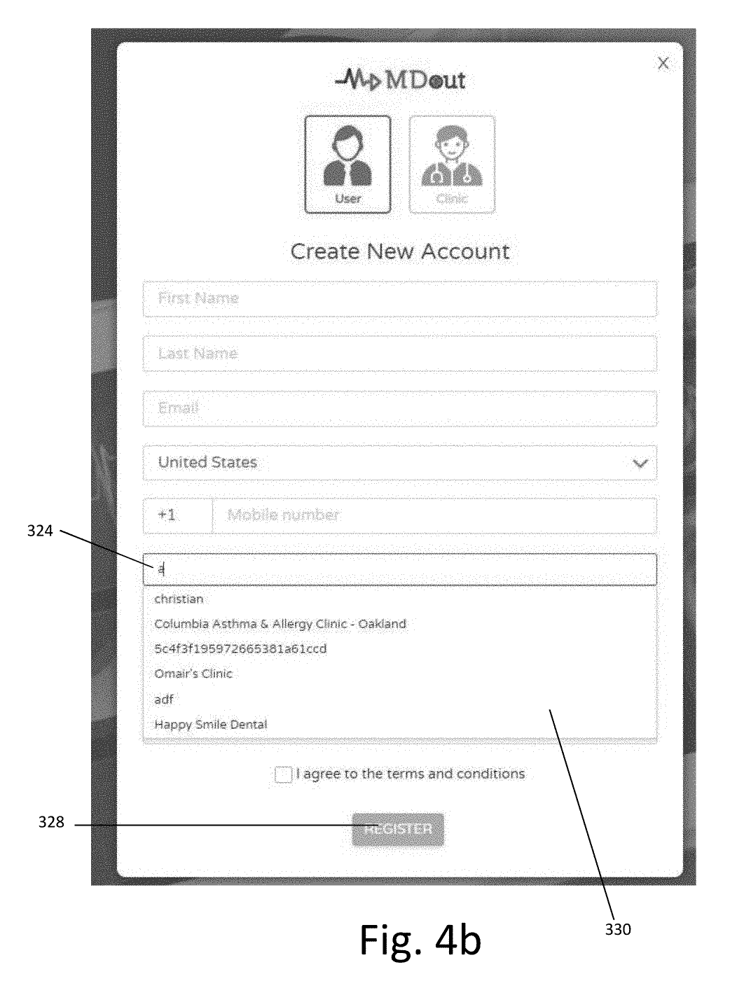

If the user is not yet registered, and wishes to do so, the user interacts with the register button 306. Interaction with the register button 306 causes the server 102 to cause the user device to display the images of FIG. 4a.

FIGS. 4a ad 4b are screenshots of images displayed by a communication device connected to the system of FIGS. 1 and 2, to enable a patient of a registered clinic to register in the system, according to some embodiments of the present invention.

If the user is a patient of a registered clinic, the user clicks on a first icon 308, and is prompted to fill basic information to send to the server. The user's basic information includes the user's first name to be typed in the text box 312, the user's last name to be typed in the text box 314, the country in which the user lives (to be selected from drop-down menu 318), the user's telephone number (to be typed in the text box 320), the name of the registered clinic (to be typed in a text box 322), and a password (to be typed in text boxes 324 and 326).

In some embodiments of the present invention, once the user starts typing the name of clinic in the text box 322, a drop-down menu 330 appears with names of clinics which include the character or characters typed. The user may select one of the clinics by interacting with the name of the relevant clinic.

Once the user has finished filling in the basic information, the user interacts with the Register button 328 in FIGS. 4a and 4b to send the basic information to the server 102. Once the basic information is sent to the server 102 and verified by the server 102 (e.g., via communication with the user's email and/or phone, as known in the art), the user's information is placed in the patient database 214. The patient may log in with the patient's credentials as shown in FIG. 3 and explained above.

If the user is the administrator of a clinic that is still not registered with the system, then the user interacts with the button 310.

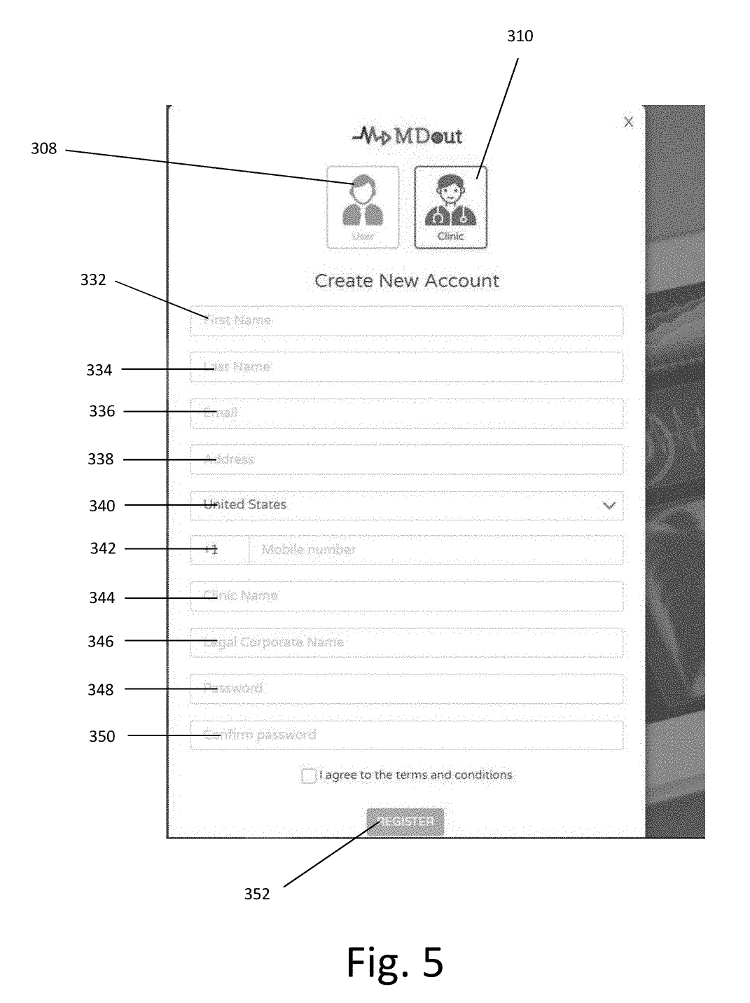

FIG. 5 is a screenshot of images displayed by a communication device connected to the system of FIGS. 1 and 2, to enable an administrator of an unregistered clinic to register the clinic in the system, according to some embodiments of the present invention.

After interacting with the button 310, the user is prompted to enter basic information of the user and the clinic. The basic information may include the first name of the clinic's administrator (to be typed in a text box 332), a last name of the clinic's administrator (to be typed in a text box 334), an email address of the clinic's administrator (to be typed in a text box 336), a physical address of the clinic (to be typed in a text box 338), a country in which the clinic is located (to be selected via a drop down box 340), a phone number of the clinic administrator (to be typed in a text box 342), a name of the clinic (to be typed in a text box 344), a name of the legal entity which owns the clinic (to be typed in a text box 346), and a password (to be typed in text boxes 348 and 350).

Once the clinic administrator has finished filling in the basic information, the user interacts with the Register button 352 to send the basic information to the server 102. Once the basic information is sent to the server 102 and verified by the server 102 (e.g., via communication with the user's email and/or phone, as known in the art, or via manual verification by a staff member of the entity that maintains the system), the information of the clinic and of the clinic's administrator is placed in the clinic database 210. The clinic's administrator may log in with the clinic administrator's credentials as shown in FIG. 3 and explained above.

FIGS. 6-38 are screenshots of images displayed by a communication device connected to the server of FIGS. 1 and 2 running the clinic management tool, according to some embodiments of the present invention. When a clinic administrator signs in, the server 102 runs the clinic management tool. All of the screenshots displayed to the clinic administrator include a first screen portion 400 designated as a dashboard, which includes links to different screens which enable the user to perform different action.

The dashboard 400 of the clinic administrator includes a first link 402, a second link 404, a third link 406, a fourth link 408, a fifth link 410, a sixth link 412, and seventh link 414. Interaction with the first link 402 directs the clinic administrator to a page which allows the clinic advisor to handle survey information. Interaction with the second link 404 directs the clinic administrator to a page which allows the clinic advisor to handle information regarding the staff of the clinic. Interaction with the third link 406 directs the clinic administrator to a page which allows the clinic advisor to handle information regarding the patients of the clinic. Interaction with the fourth link 408 directs the clinic administrator to a page which allows the clinic advisor to manage the permissions given to different types of staff members of the clinic. Interaction with the fifth link 410 directs the clinic administrator to a page which allows the clinic advisor to create and edit content (videos and articles). Interaction with the sixth link 412 allows the clinic administrator to view information relating to videos available to the clinic's patient, which may have been added by the clinic's administrator or by a different user (e.g., the support administrator). Interaction with the seventh link 414 allows the clinic administrator to view information relating to articles available to the clinic's patient, which may have been added by the clinic's administrator or by a different user (e.g., the support administrator).

FIG. 6 is a screenshot of an introduction screen displayed to the clinic administrator. The introduction screen has the first (dashboard) portion 400, as described above. The introduction screen also includes a clinic link 415 (which may also be present in all the screens shown to the clinic administrator). Interaction with the clinic link allows the clinic administrator to view and edit information relating to the clinic.

In some embodiments of the present invention, the introduction screen includes one or more summary windows. Each window occupies a respective portion of the screen distinct from other windows. A survey summary window 416 displays the number of surveys available. A survey completion summary window 418 displays the number of surveys that have been completed by patients. A pending survey window 420 displays the number of surveys that have been assigned to patients of the clinic and have not been completed yet. A patient summary window 422 displays the number of patients of the clinic registered in the system. A staff summary window 424 displays the number of staff members of the clinic registered in the system.

In some embodiments of the present invention, the introduction screen includes a patient list window 426 and a staff list window 428. The patient list window 426 displays a list of at least some of the patients of the clinic. The order in which the patients are listed may be random, alphabetical (by last name), according to the latest log in of the patient (e.g., latest patient to log in is showed ahead of patients who have logged in at an earlier time), according to the latest creation of the patient (e.g., latest patient to be added to the system is showed ahead of patients who have been added at an earlier time).

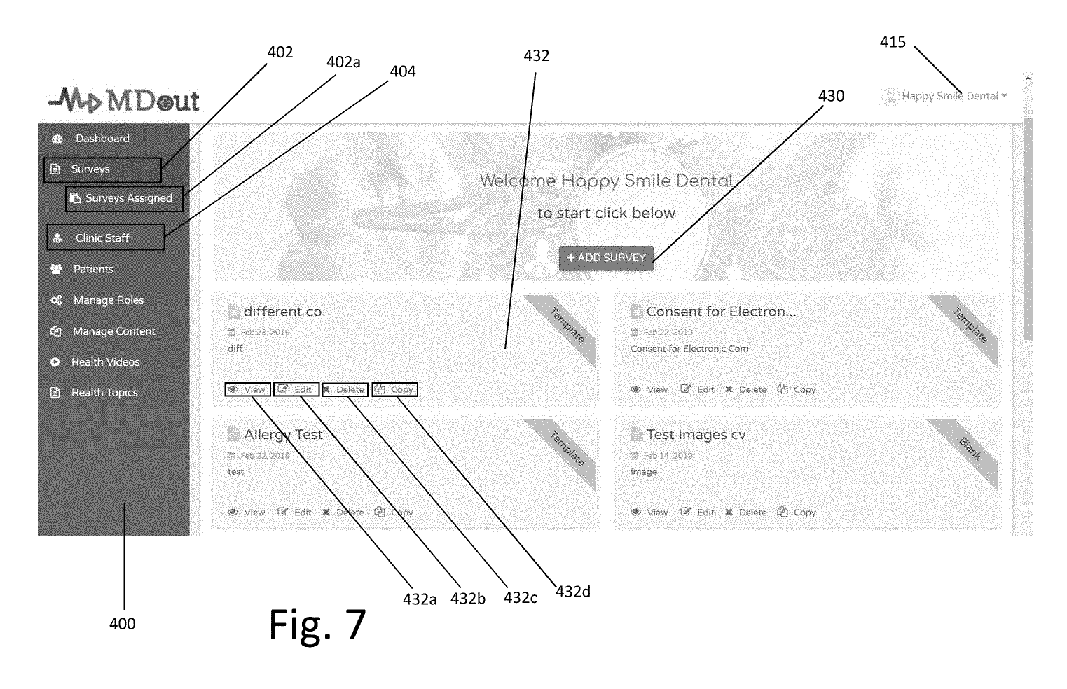

FIG. 7 is a screenshot of a survey page displayed to the clinic administrator after the clinic administrator has interacted with the first link 402. In the page of FIG. 7, a sub-link 402a appears in the dashboard 400. The sub-link 402a is optionally located between the first link 402 and the second link 404.

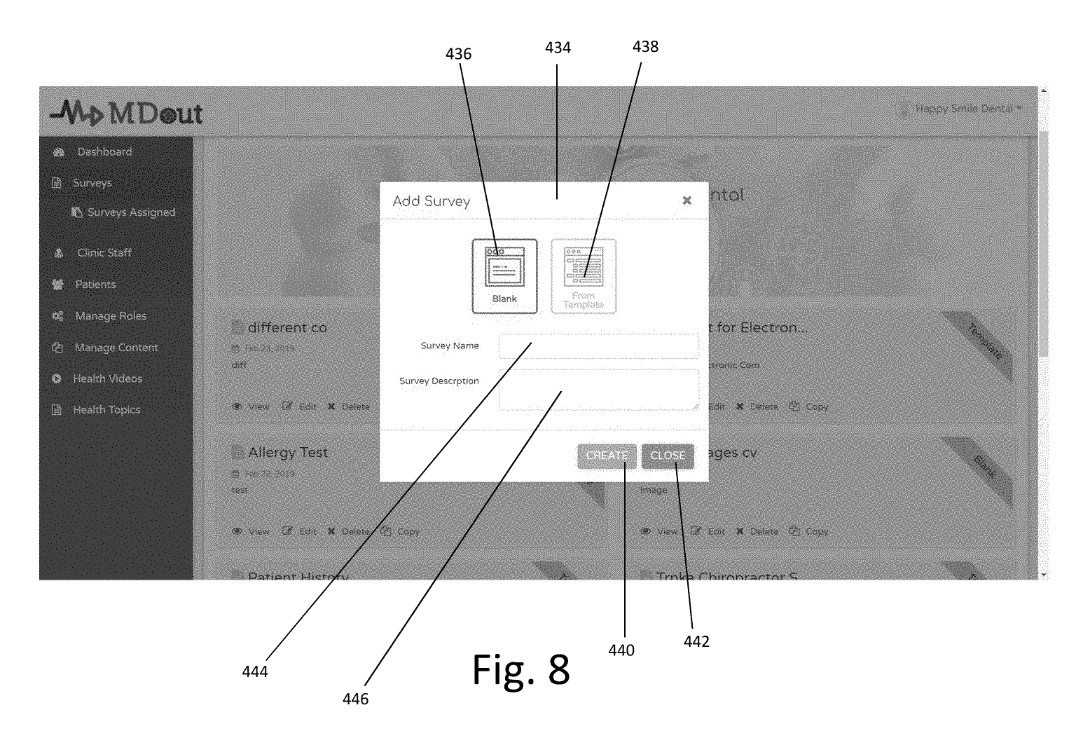

The survey page includes the dashboard 400 and optionally the clinic link as 415 described above. The survey page further includes an “add survey” button 430. Interacting with “add survey” button directs the clinic administrator to a series of pages with tools configured to enable the clinic administrator to input instructions and information necessary for creating a survey, as will be seen further below, in the description of FIGS. 8-13.

In some embodiments of the present invention, the survey page includes survey summary windows 432. Each survey summary window 432 is associated with a respective survey and includes a title of the survey, a short description of the survey, and at least three buttons: a view button 432a, and edit button 432b, and a delete button 432c. Optionally, the survey summary window 432 also includes a copy button 434d.

By interacting with the view button 432a in the survey summary window 432 associated with a desired survey, the clinic administrator may view the survey as it would be viewed by a patient filling the survey. By interacting with the edit button 432b, the clinic administrator is directed to one or more pages with tools configured to enable the clinic administrator to edit the survey. By interacting with the delete button 432c in the survey summary window 432 associated with a desired survey, the clinic administrator may delete the survey. By interacting with the copy button 432d in the survey summary window 432 associated with a desired survey, the clinic administrator may create a new survey that is a copy of the desired survey.

In FIG. 8, a pop-up window appears 434 above the previous screen, after the clinic administrator has interacted with the “add survey” button 430 of FIG. 7.

The pop-up window 434 includes a “blank” button 436, a “template” button 438, a “create” button 440, and a “close” button 442. The clinic administrator can select the “blank” button 436 to create a survey from scratch or the “template” button 438 to create a survey from an existing template. If the “blank” button 436 is selected, two text boxes appear in the pop-up window 434: a first text box 444 for enabling the user to type a name of the survey, and a second text box 446 for enabling the user to type a description of the survey.

Interacting with the “create” button causes the text input by the user in the text boxes 444 and 446 to be received by the server, and causes the clinic administrator to be directed to one or more pages configured to enable the clinic administrator to create and customize a survey. Interacting with the “close” button 442 causes the pop-up window 434 to close and returns to the page behind the pop-up window 434.

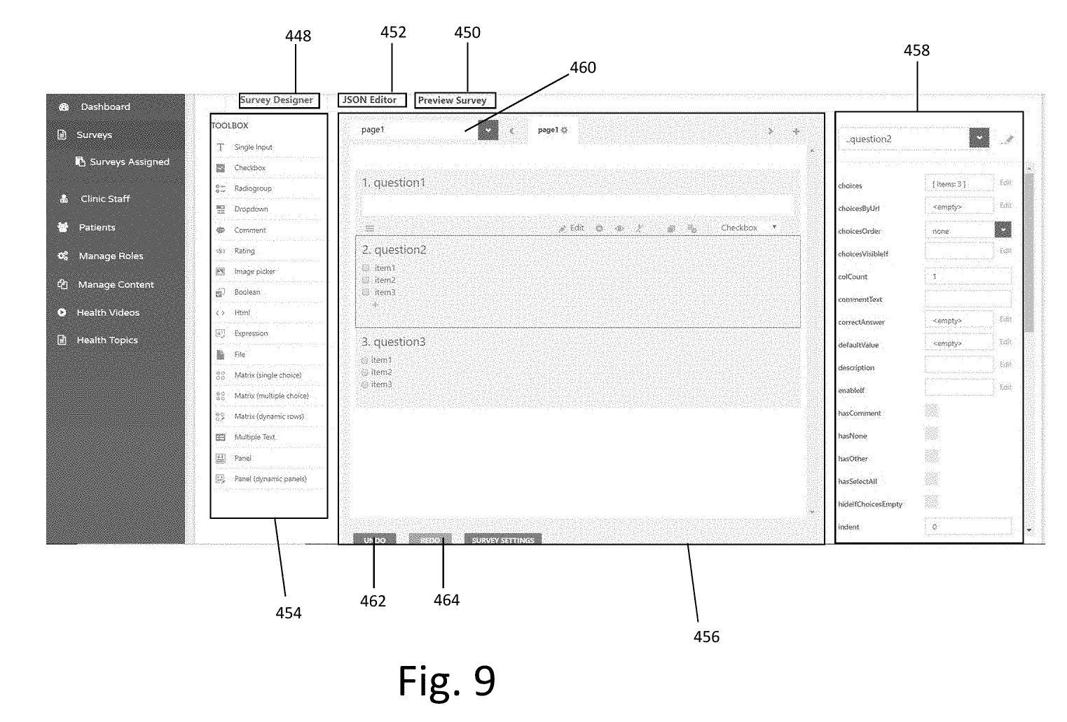

After interacting with the “create” button of FIG. 8, the survey creation screen is displayed, as shown in FIG. 9. The survey creation screen includes a first tab 448, a second tab 450, and optionally a third tab 452. When the first tab 448 is interacted with, a toolbox window 454, a survey body window 456, and optionally a question format window 458 are displayed.

The toolbox window 454 includes a plurality of portions associated with respective types of inputs that questions may be answered with, such a single (text) input question, a checkbox, a radio group, selection from a dropdown menu, etc. Interaction (e.g., by clicking, touching, etc.) with a portion of the toolbox associated with a desired answer type causes a question and answers of the desired type to appear (be displayed) in the survey body window 456. The clinic administrator may enter more questions into the survey body window 456, by interacting again with the toolbox window 454. In the survey body window, the clinic administrator may type the text of each question and of each answer (if the answers are predetermined) and may add answers to different types of multiple-choice answers.

A specific question may be selected in the survey body window 456 by interacting with the section of the display in which the question is displayed. When a specific question is selected in the survey body window 456, a question format window specific 458 to the selected question may be displayed. In the question format window, different choices about the characteristic of the selected question and the questions answer are displayed and may be selected, by interaction with the display sections in which the choices are displayed. Selecting any specific choice varies the question and/or the question's answers in the survey body window 456.

Optionally, the survey body window includes a page selection tool 460. A survey may be generated to have many pages, and by selecting the appropriate pages in the page selection tool 460 before interacting with the toolbox window 454, the clinic administrator can place a desired question on a desired page of the survey.

Optionally, the survey body window includes an “undo” button 462 and a “redo” button 464. Interaction with the “undo” button 462 causes the server to undo the last action performed on the screen of FIG. 9. Interaction with the “redo” button 464 causes the server to redo the last undone action performed on the screen of FIG. 9.

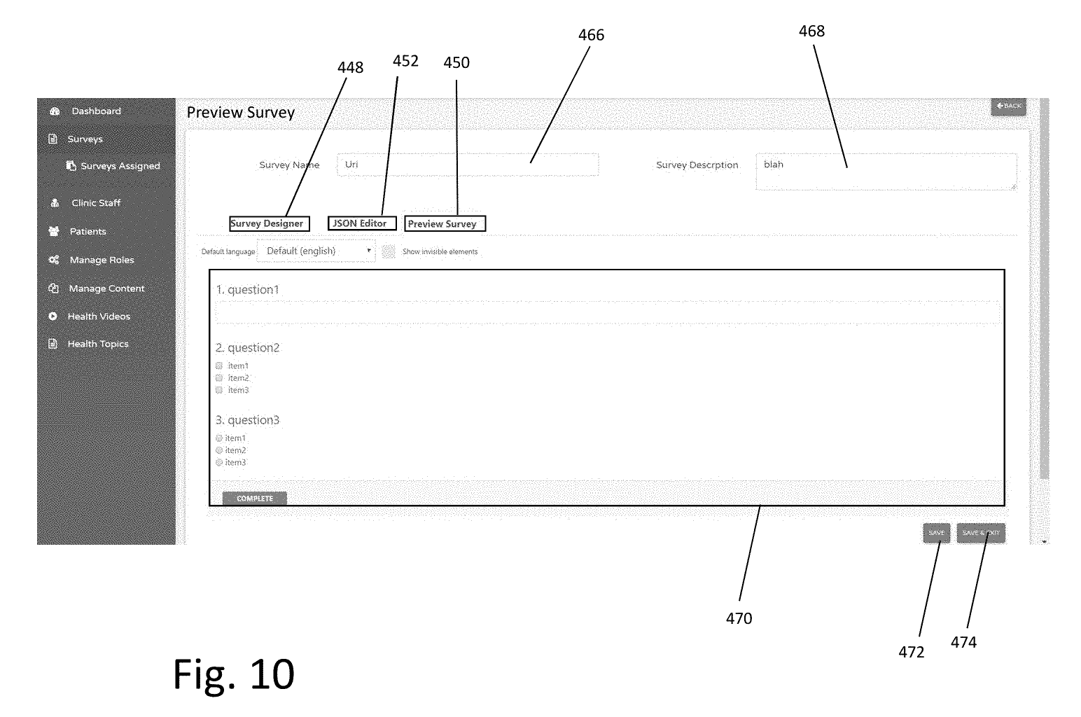

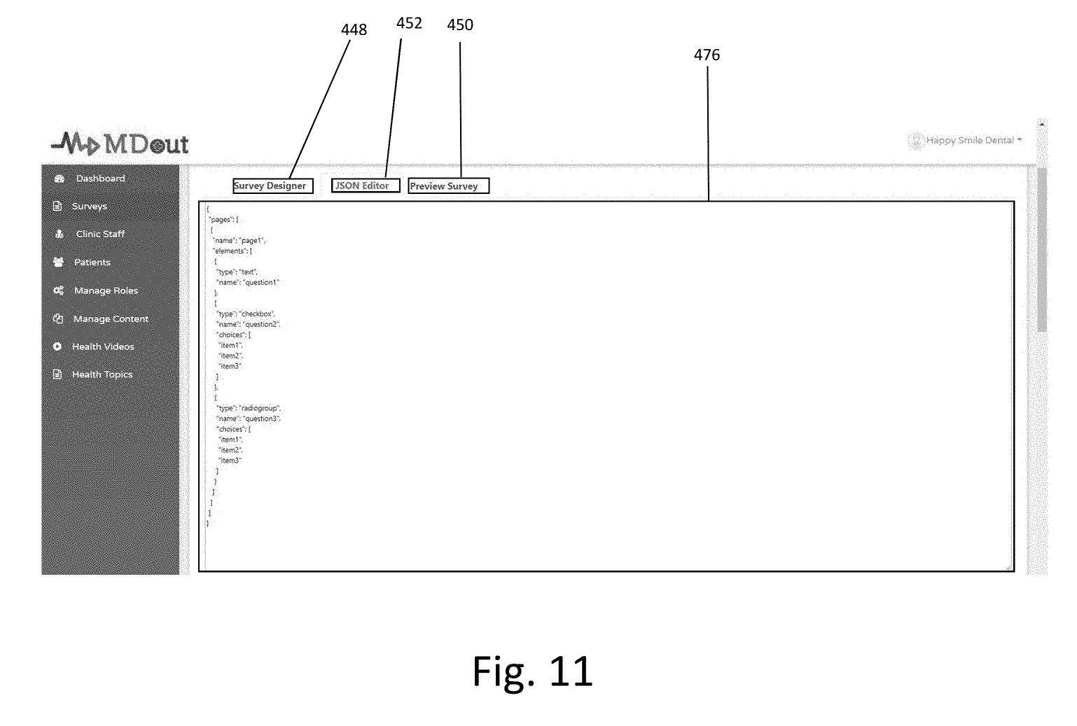

Interacting with the second tab 450 causes the server to display a preview of the survey on a preview page, as seen in FIG. 10. Interacting with the third tab 452 causes the server to display a programming editor page in which the clinic administrator may type commands in a computer language, for causing the server to create the survey, as seen in FIG. 11. In the screens of FIGS. 10 and 11, the tabs 448, 450, and optionally 452, are displayed, to enable the user to go back and forth between the pages of the FIGS. 9, 10, and 11 as needed.

The preview page of FIG. 10 includes a text box 466 with the name of the survey as entered in FIG. 8, a text box 468 with the description of the survey as entered in FIG. 8, a survey preview window 470 which displays the survey as it would be seen by a patient, a “save” button 472, and a “save and exit” button 474. Interaction with the “save” button 472 causes the survey to be saved in the server for later use. Interaction with the “save and exit” button 474 causes the survey to be saved in the server for later use and returns to the survey page of FIG. 7.

The programming editor page of FIG. 11 includes a code box 476 in which the clinic administrator may type commands in a programming language, for causing the server to create the survey, as seen in FIG. 11. In some embodiments of the present invention, the programming language is JSON. Other languages such as HTML, C, etc., may be used instead.

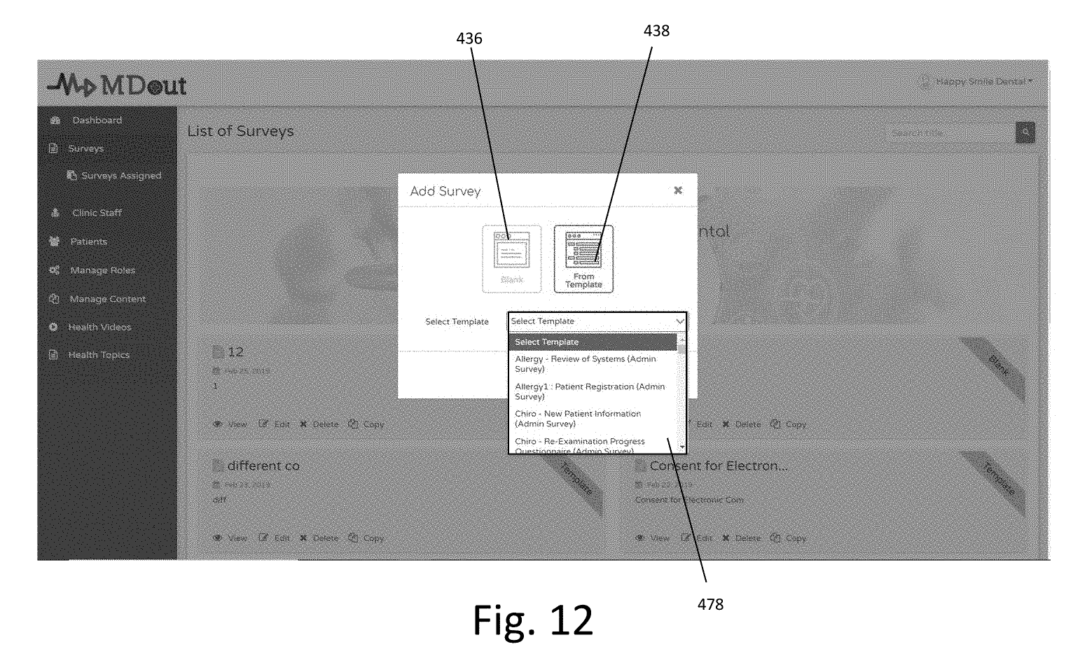

FIG. 12 is a screen shot of a survey creation pop-up window that is displayed if the template button 438 is selected in FIG. 8.

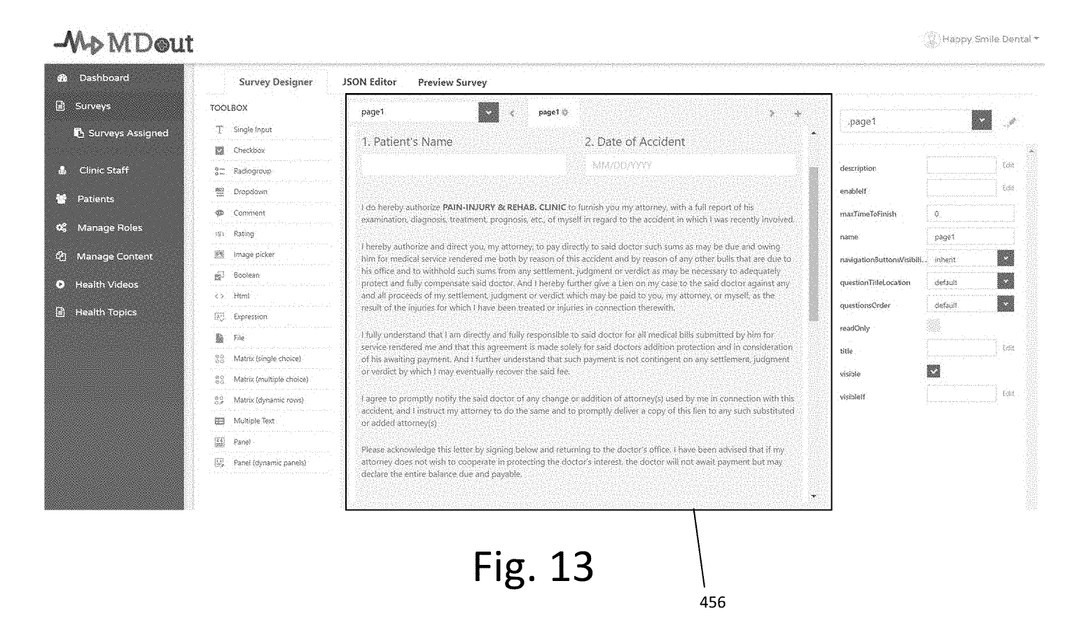

A list 478 of pre-determined templates is displayed (optionally as drop down menu), so the clinic administrator can choose the template by selecting a desired template. Once the desired template is selected, a survey creation page akin to the one of FIG. 9 is displayed, with the survey body 456 pre-filled with text, questions, and answers from the survey template. This may be seen in FIG. 13. The template may serve as a starting point for adding more questions and answers to the new survey being created. It should be noted that the survey creation page of FIG. 13 associated with an existing survey is also displayed when the when the clinic's administrator interacts with the “edit” button 432b of FIG. 7.

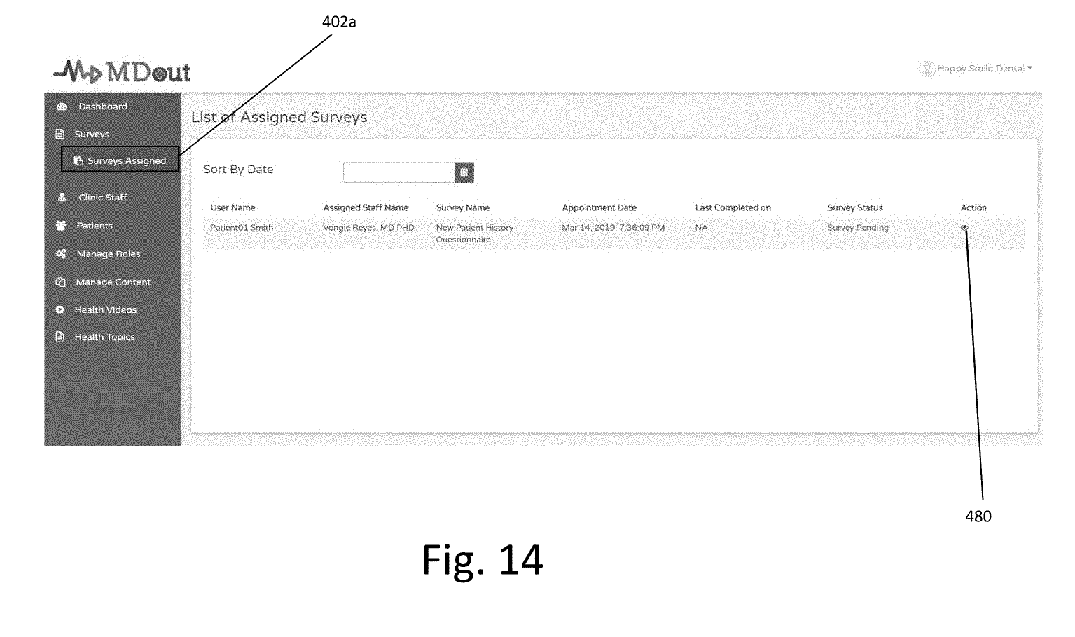

FIG. 14 is a screen shot of a list of surveys assigned to patients that have not been completed. In some embodiments of the present invention, some data relating to each survey in the list is displayed near the survey. The data may include the name of the patient, the name of the survey, the name of the clinic's staff member assigned to the patient, the date of the patient's appointment at the clinic, and the status of the survey (completed or pending). Optionally, the clinic administrator may view the survey by interacting with the eye icon.

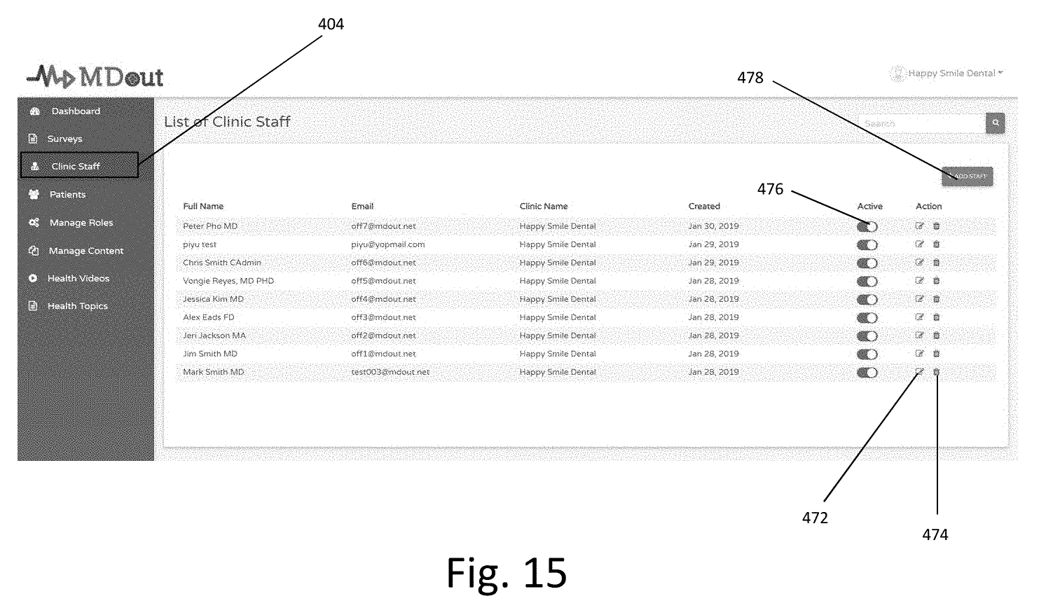

FIG. 15 is a screenshot of a list of staff members of the clinic. The list is displayed when the clinic's administrator interacts with the link 404.

A respective line is assigned to each staff member. In the list, at least the name of each staff member is listed along with an “edit” icon 472 and a “delete” icon 474. Other information may be displayed, such as the email address of the staff members, and the date in which the staff members were entered into the system (created).

In some embodiments of the present invention, an activity icon 476 may be interacted with to designate the staff member as active or passive. The activity icon has two configurations: an “on” configuration indicating that the staff member is active and an “off” configuration indicating that the staff member is passive. Interacting with the activity icon switches the icon from one configuration to the other.

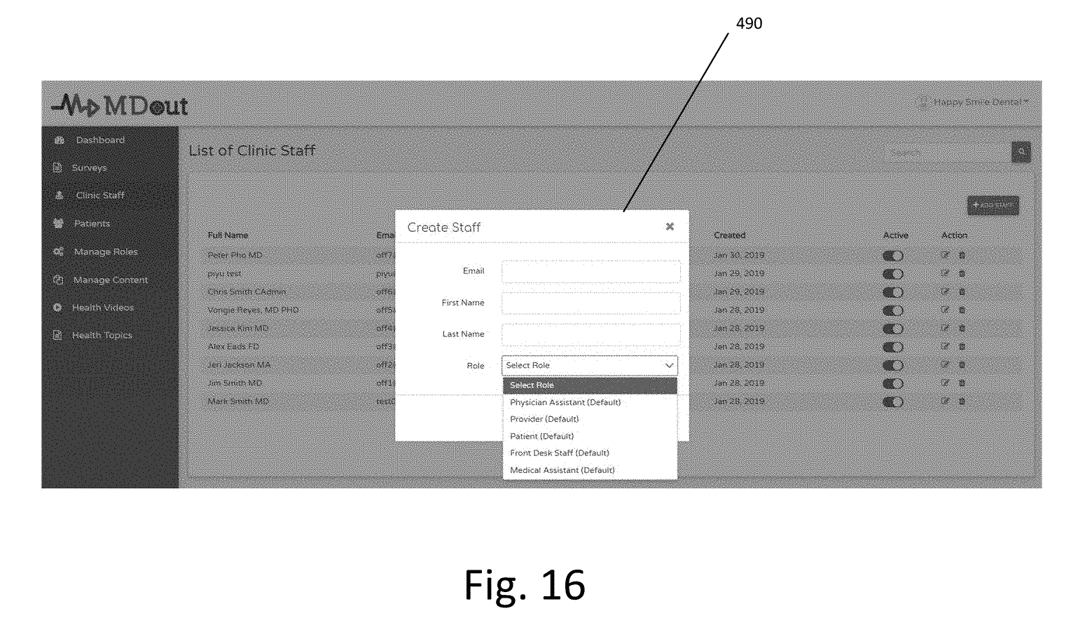

A “create staff” button 478 is also displayed. Interaction with the “create staff” button 478 causes the server to display one or more pages with prompts that enable the user to enter a staff member in the staff database, as will be seen in FIG. 16.

FIG. 16 is a screenshot of a prompt 490 that enables the user to create a new staff member in the database. The prompt is displayed in the center of the screen, thereby reducing the list of staff members to the background.

The prompt 490 for creating a staff member allows the user to enter the new member's email address, first name, last name, and role. There is a drop down list comprising various roles that the user may select from. For example, physician assistant, provider, patient, front desk staff, and medical assistant. Each role is associated with respective permissions to perform different actions.

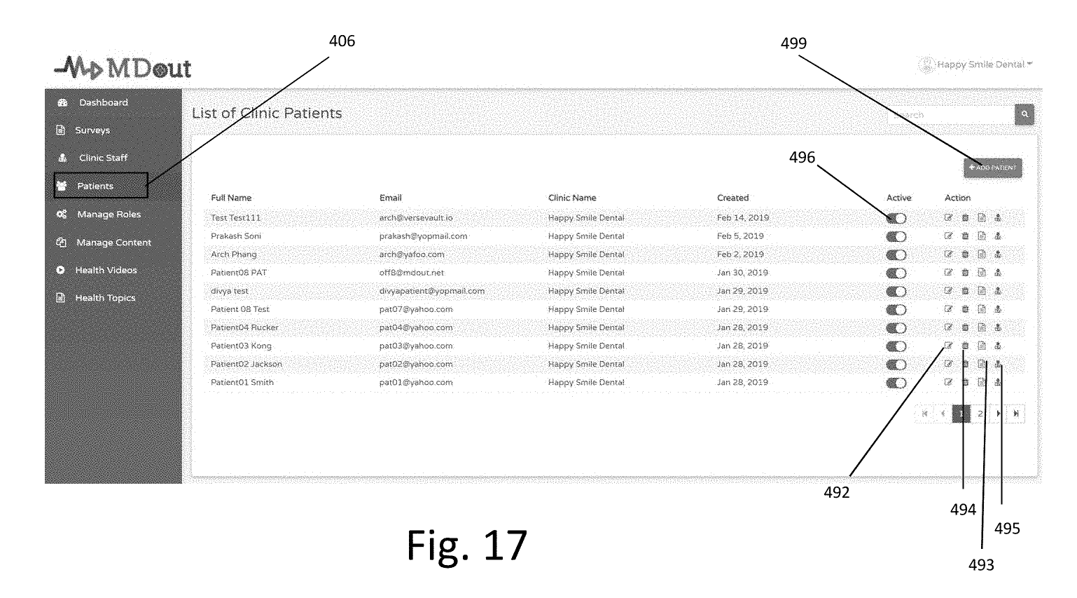

FIG. 17 is a screenshot of a list of patients at the clinic. The list is displayed when the clinic's administrator interacts with the “patients” link 406 on the left side of the screen.

A respective line is assigned to each patient. In the list, at least the name of each patient is listed along with an “edit” icon 492 a “delete” icon 494, an “assign survey” icon 493, and an “assign provider” icon 495. Other information may be displayed, such as the email address of the patients, and the date in which the staff members were entered into the system (created).

In some embodiments of the present invention, an activity icon 496 may be interacted with to designate the patient as active or passive. The activity icon has two configurations: an “on” configuration indicating that the patient is active and an “off” configuration indicating that the patient is passive. Interacting with the activity icon switches the icon from one configuration to the other.

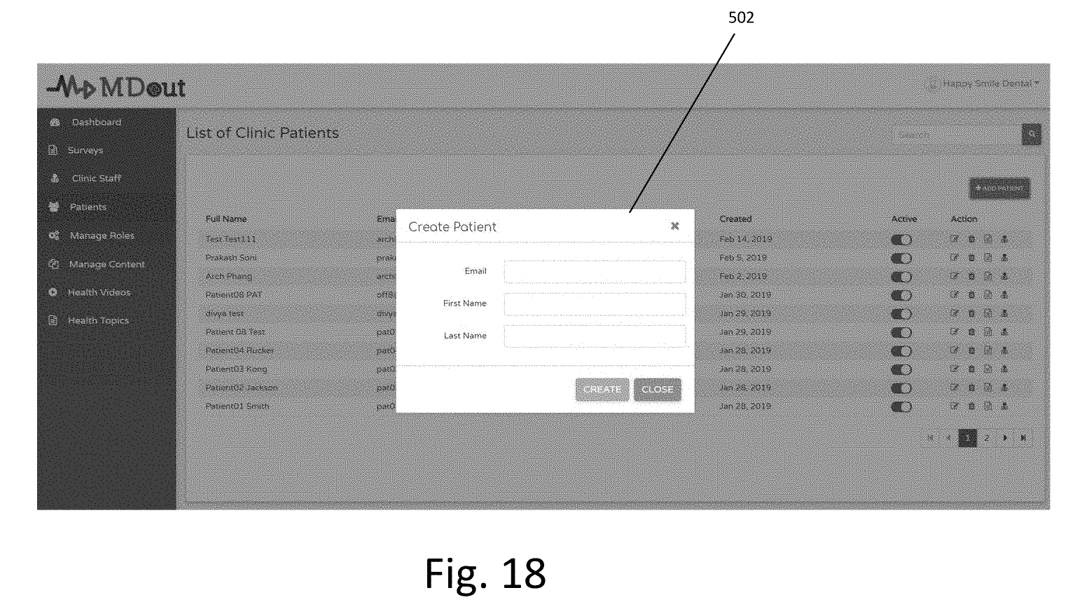

An “add patient” button 499 is also displayed. Interaction with the “add patient” button 499 causes the server to display one or more pages with prompts that enable the user to enter a patient in the patient database, as will be seen in FIG. 18.

FIG. 18 is a screenshot of a prompt 502 that enables the user to create a new patient in the database. The prompt is displayed in the center of the screen, thereby reducing the list of patients to the background.

The prompt 502 for creating a patient allows the user to enter the new patient's email address, first name, and last name.

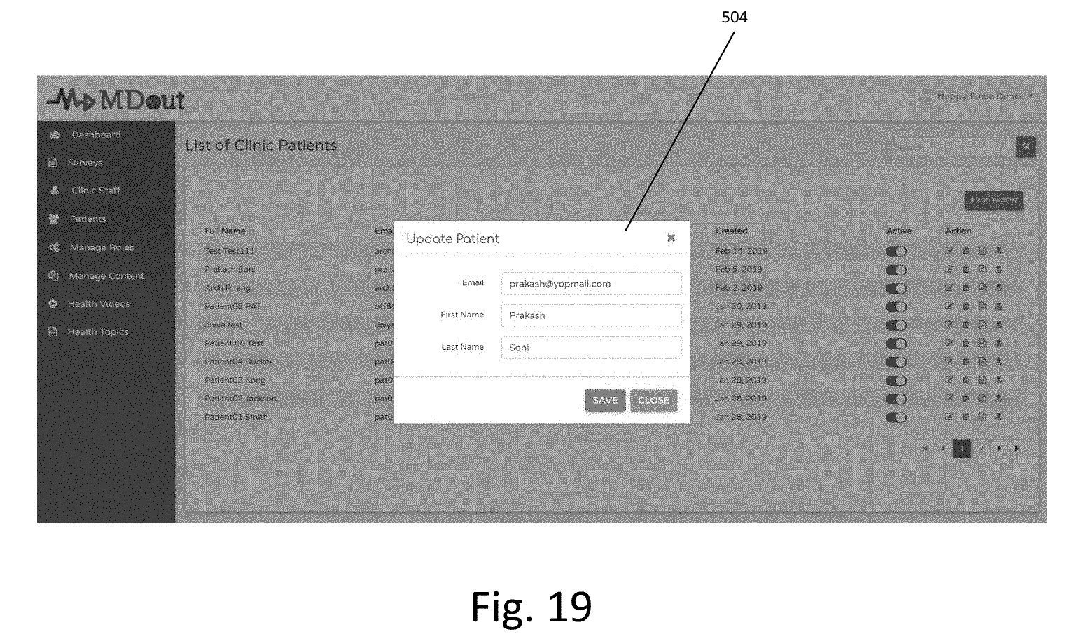

FIG. 19 is a screenshot of a prompt 504 that enables the user to update a patient's information. Interaction with the “edit” icon 492 of FIG. 17 causes the server to display this prompt 504. The prompt 504 is displayed in the center of the screen, thereby reducing the list of patients to the background.

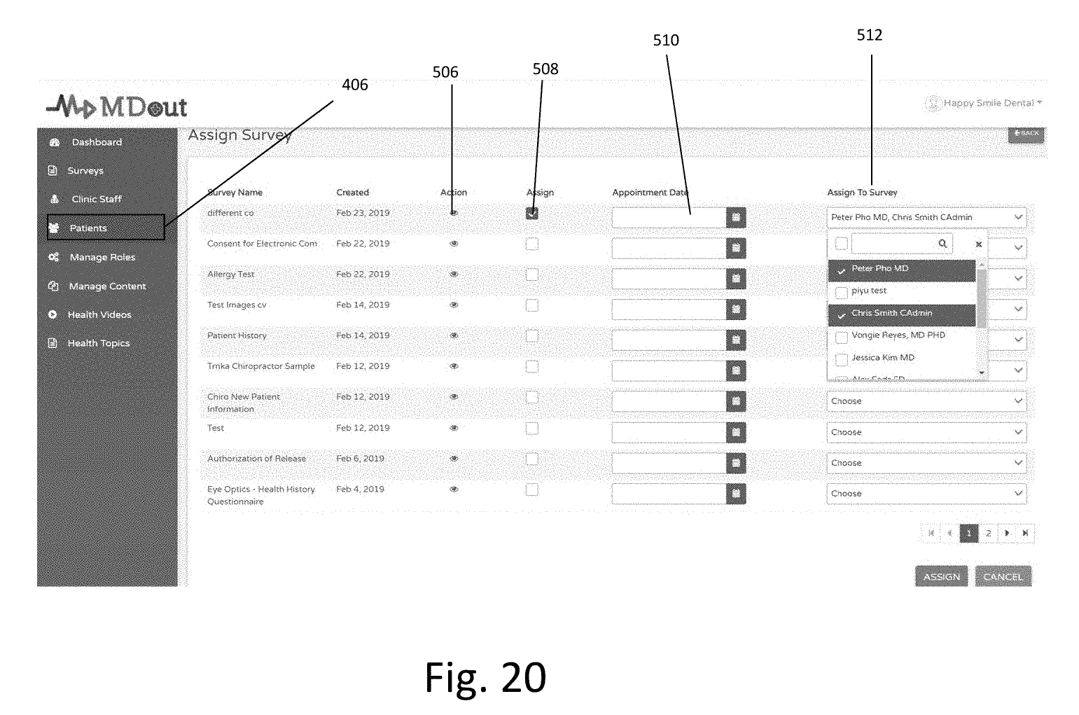

FIG. 20 is a screenshot of a list of surveys at the clinic. The list is displayed when the clinic's administrator interacts with the “assign survey” icon 493 next to a patient's name in the list of patients of FIG. 17.

A respective line is assigned to each survey. In the list, at least the name of each survey is listed along with an “action” icon 506, an “assign” icon 508, an “appointment date” icon 510, and an “assign to survey” icon 512. Other information may be displayed, such as the date in which the surveys were entered into the system (created).

Interaction with the “action” icon 498 displays the survey to the clinic's administrator. The “appointment date” icon 510 enables the clinic's administrator to select a date from a calendar. The date specifies the patient's next appointment date with the provider (staff member of the clinic). The “assign to survey” icon 502 enables the clinic administrator to select from a drop down list of providers (staff members of the clinic), to assign the survey for one or more of the providers.

In some embodiments of the present invention, each time a new survey is assigned to a patient, the server sends a message to the patient via the patient's contact information (e.g., email, text message to a phone, etc.). The message informs the patient that a new survey/questionnaire has been assigned to the patient.

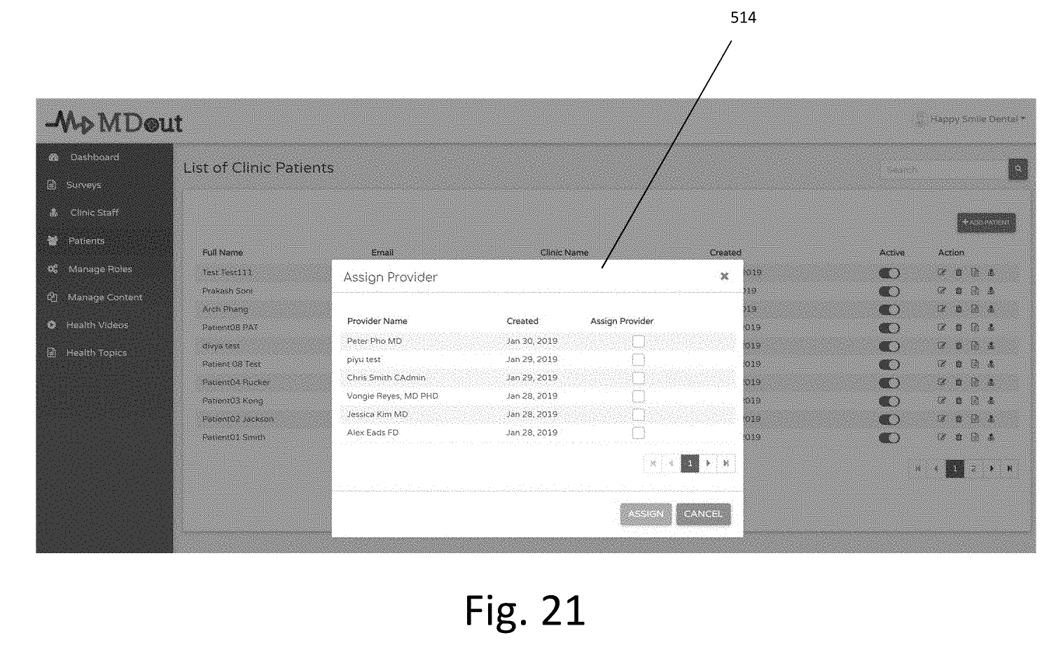

FIG. 21 is a screenshot of a prompt 514 that enables the clinic's administrator to select one or more providers (staff members of the clinic) from a list of providers to be associated with a selected patient. Interaction with the “assign provider” icon 495 of FIG. 17 causes the server to display this prompt 514. The prompt 514 is displayed in the center of the screen, thereby reducing the list of patients to the background.

A respective line is assigned to each provider. In the list, at least the name of each provider is listed along with an option to select a provider and the date in which the provider was entered into the system (created).

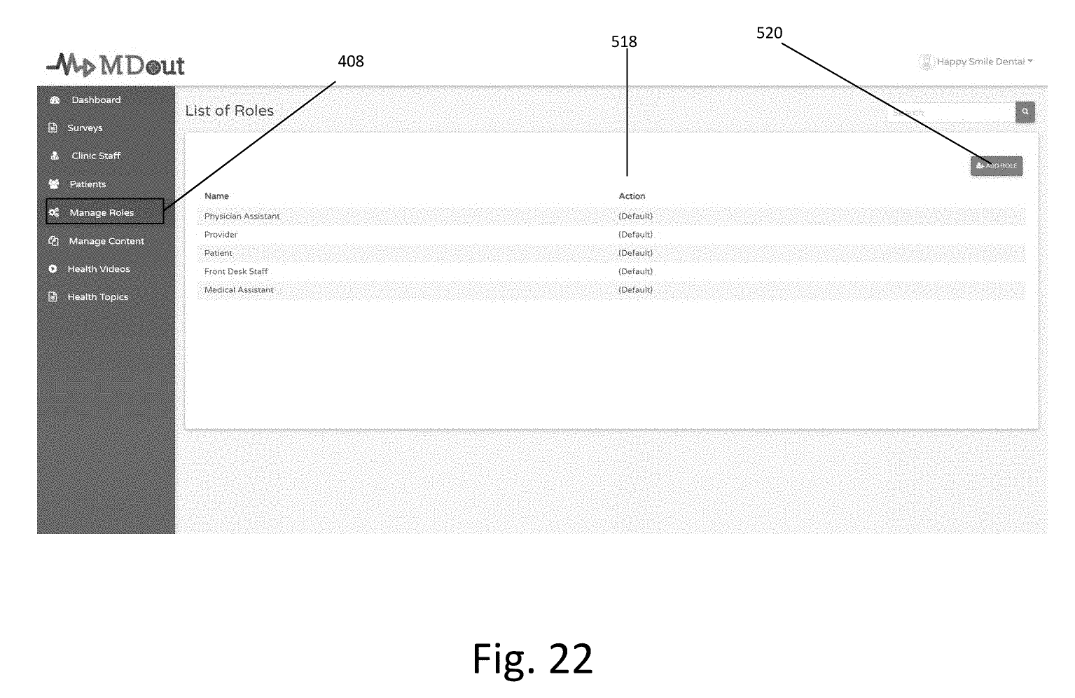

FIG. 22 is a screenshot of a list of roles at the clinic. The list is displayed when the clinic's administrator interacts with the “manage roles” link 408 on the left side of the screen.

A respective line is assigned to each role. In the list, at least the name of each role is listed along with an “action” icon 518.

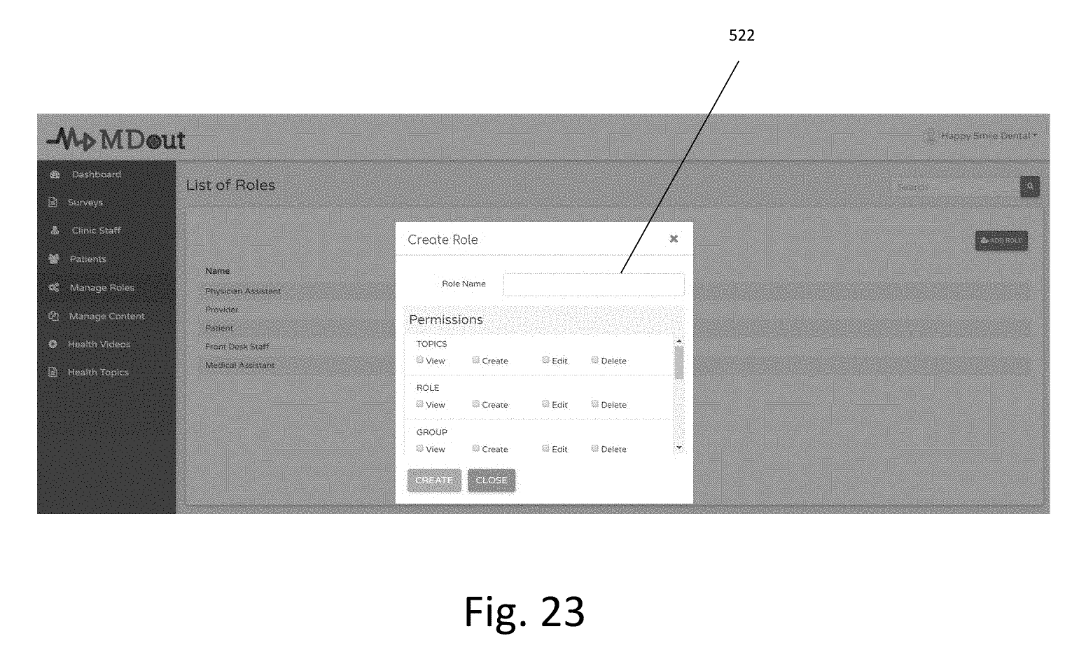

An “add role” button 520 is also displayed. Interaction with the “add role” button 520 causes the server to display one or more pages with prompts that enable the user to enter a role in the database, as will be seen in FIG. 23.

FIG. 23 is a screenshot of a prompt 522 that enables the user to create a role in the database. Interaction with the “add role” button 520 of FIG. 22 causes the server to display this prompt 522. The prompt 512 may be displayed in the center of the screen, thereby reducing the list of roles to the background.

The prompt 522 enables the user to select the extent of access to management of the database that the new role has. The permission access comprises topics, role, group, video, settings, clinic staff, survey, and patient. Within each category, the user further selects whether the new role has access to view, create, edit, and/or delete.

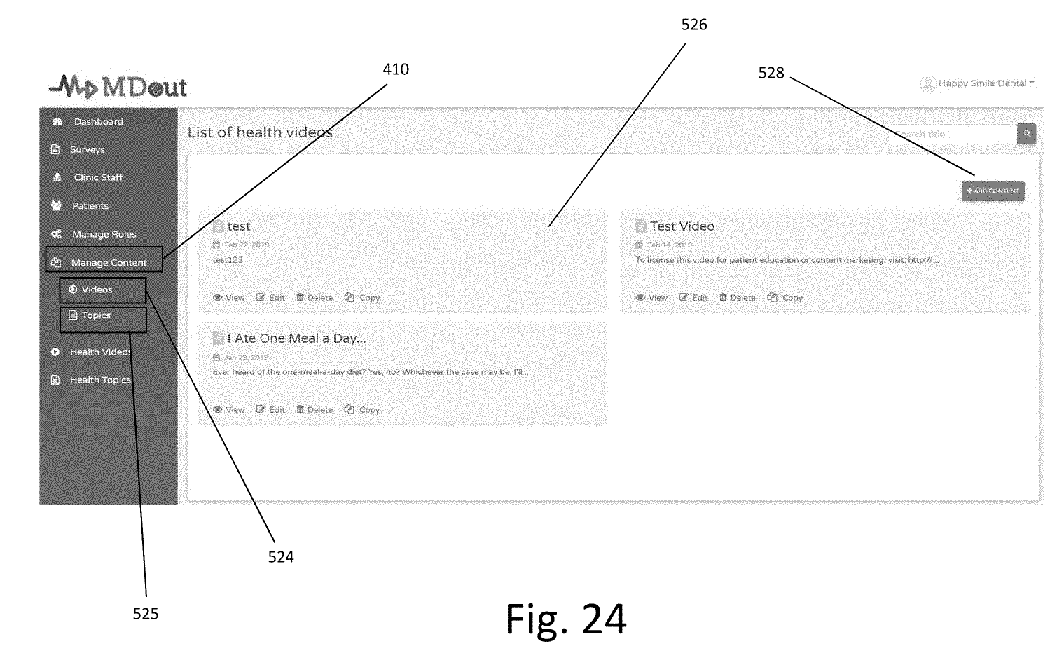

FIG. 24 is a screenshot of a list of health videos. When the “manage content” link 410 is interacted with, a “videos” sublink 524 and “topics” 525 are displayed under the “manage content” link 410 of the dashboard window. The list is health videos is displayed when the clinic's administrator interacts with the “videos” sublink 524.

A respective video summary window 526 is assigned to each video. In the video summary window 526, at least the name of each video is listed along with icons such as “view,” “edit,” “delete,” and “copy.” Other information may be displayed, such as the date in which the video was entered into the system (created) and the beginning of the description of the video.

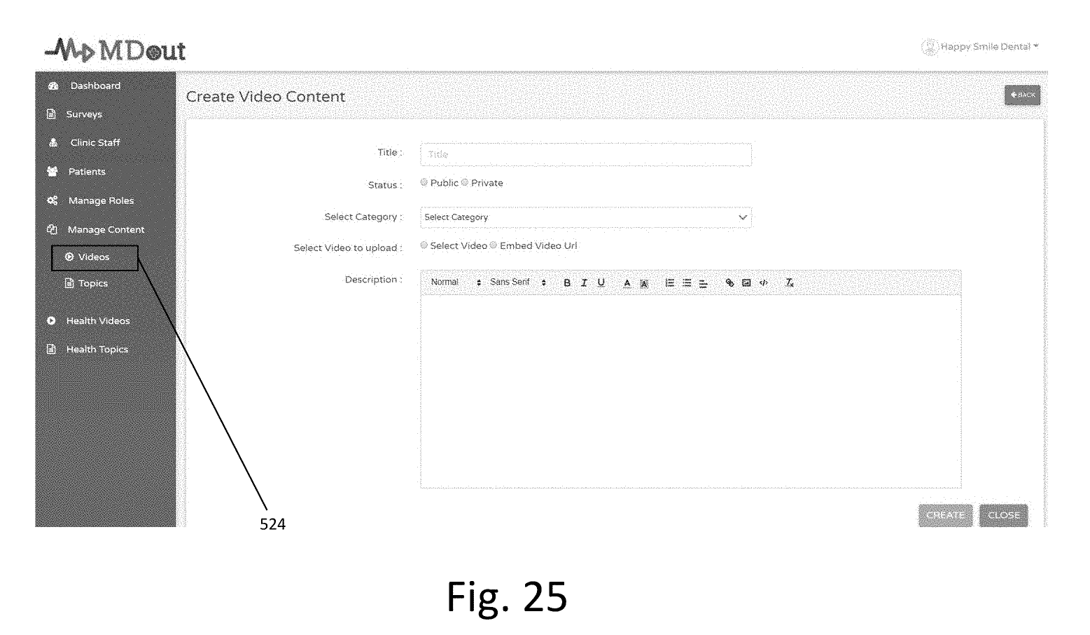

An “add content” button 528 is also displayed. Interaction with the “add content” button 528 causes the server to display one or more pages that enable the user to create a video in the database, as will be seen in FIG. 25.

FIG. 25 is a screenshot of a page that enables the user to create a video to add to the video database. Interaction with the “add content” button 528 of FIG. 24 causes the server to display this page.

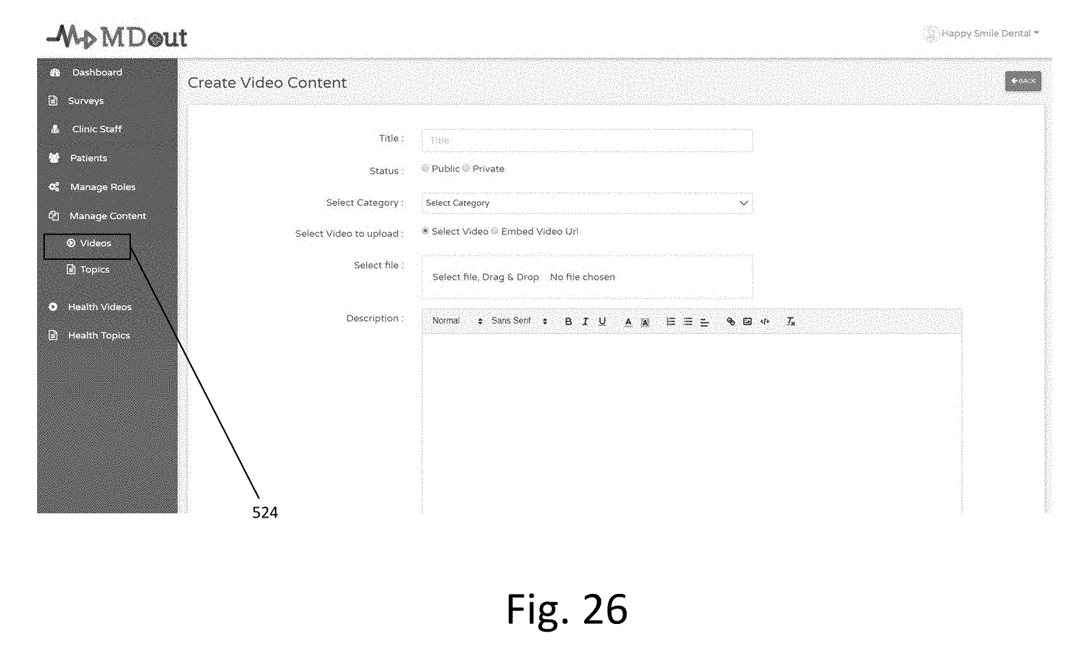

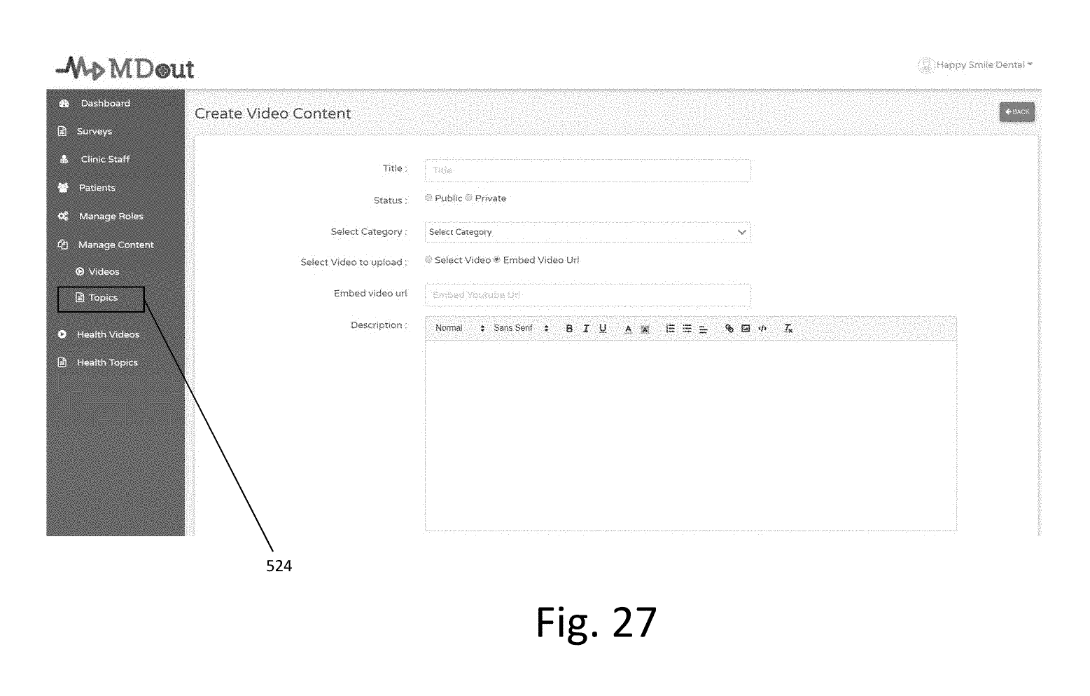

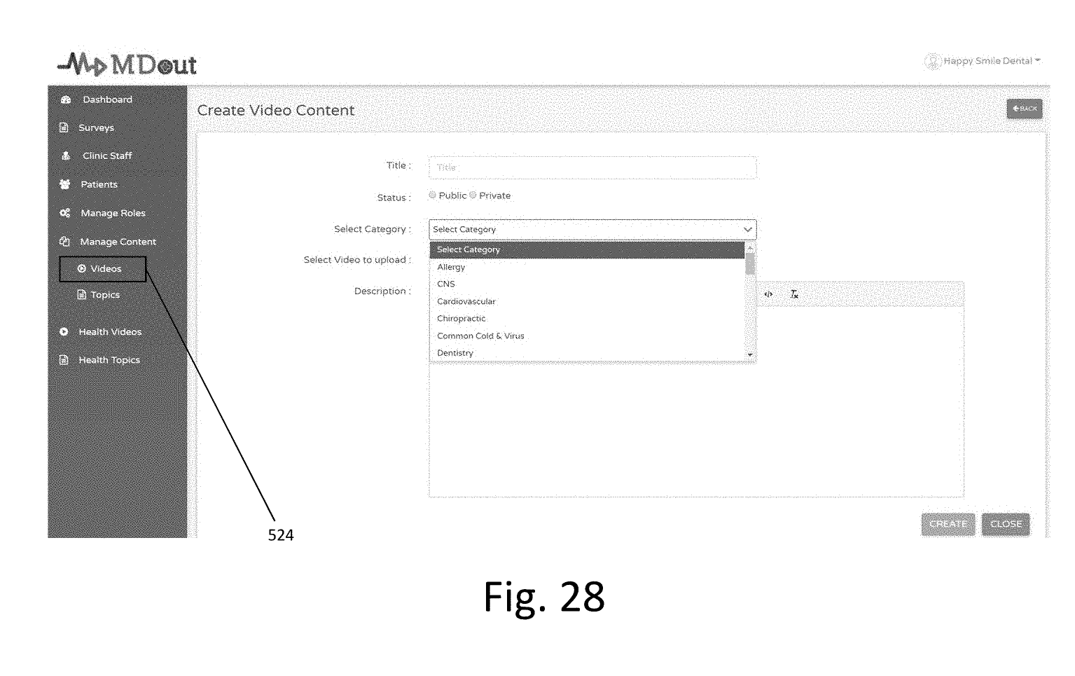

The page enables the clinic's administrator to create a title for the video and to select whether the video is public or private. A private video is viewable by patients and staff of the clinic. A public video is meant to be viewable by all users registered in the server, and needs to be approved by a system's administrator. The clinic's administrator may also select a category in which to place the video and provide a description of the video, as further seen in FIG. 28. The page enables the user to further choose whether to “select video” (i.e. upload a video stored on the clinic administrator's device, or “embed video URL,” as will be seen in FIGS. 26-27.

In some embodiments of the present invention, each time a private video is added by the clinic's administrator, the server sends a message to all patients associated with the clinic that a new video has been added.

FIG. 26 is a screenshot of the page after the user has chosen the option to “select video.” Interaction with the “select video” option causes the server to display a “select file” category, with an option to select a file or to drag and drop a file into the resulting box.

FIG. 27 is a screenshot of the page after the user has chosen the option to “embed video URL.” Interaction with the “embed video URL” option causes the server to display an “embed video URL” category, with an option to insert the URL into the resulting box.

FIG. 28 is a screenshot of the page after the user has selected the drop down menu for “select category.” Some of the categories from which the user may choose from include allergy, CNS, cardiovascular, chiropractic, common cold and virus, and dentistry.

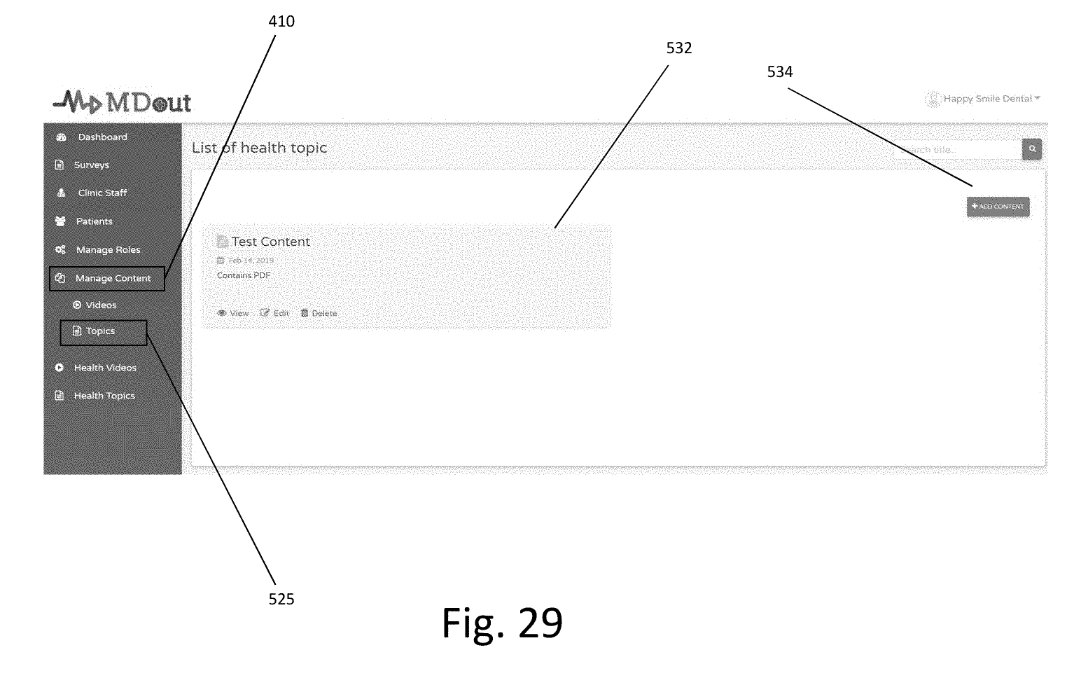

FIG. 29 is a screenshot of a list of health topics. The list is displayed when the clinic's administrator interacts with the “topics” link 525 under the “manage content” link 410 on the dashboard window. Topics are written articles.

A respective summary window 532 is assigned to each topic. In the summary window 532, at least the name of each topic is listed along with icons such as “view,” “edit,” and “delete.” Other information may be displayed, such as the date in which the topic was entered into the system (created) and a description of the topic.

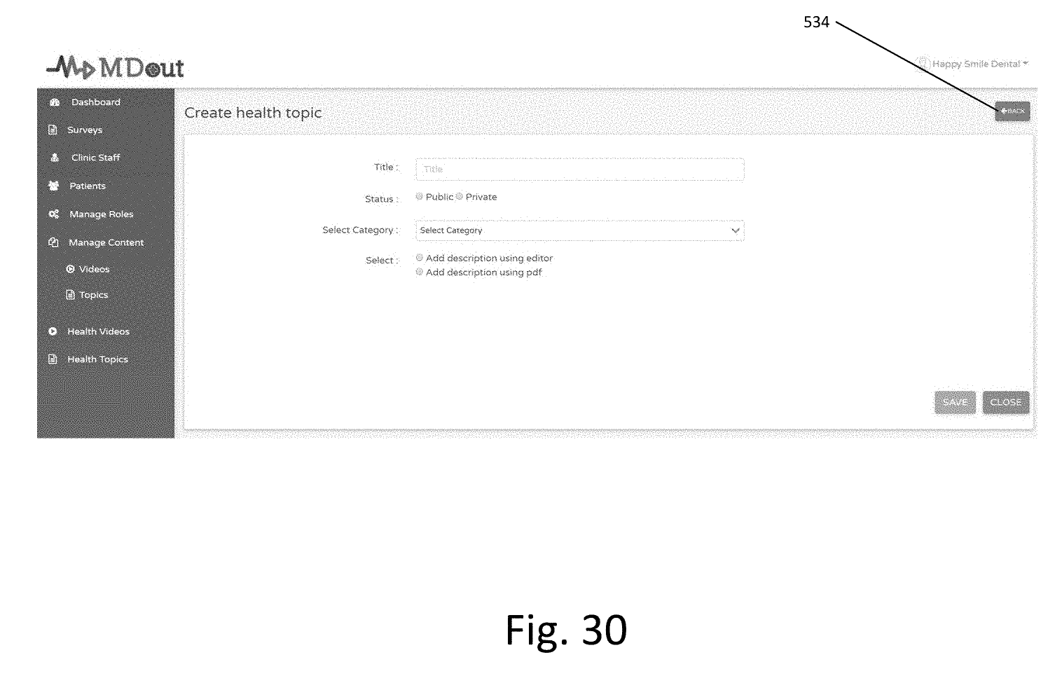

An “add content” button 534 is also displayed. Interaction with the “add content” button 534 causes the server to display one or more pages that enable the user to create a topic in the database, as will be seen in FIG. 30.

FIG. 30 is a screenshot of a page that enables the clinic administrator to create a health topic in the database. Interaction with the “add content” button 534 of FIG. 29 causes the server to display this page on the screen of clinic administrator's device.

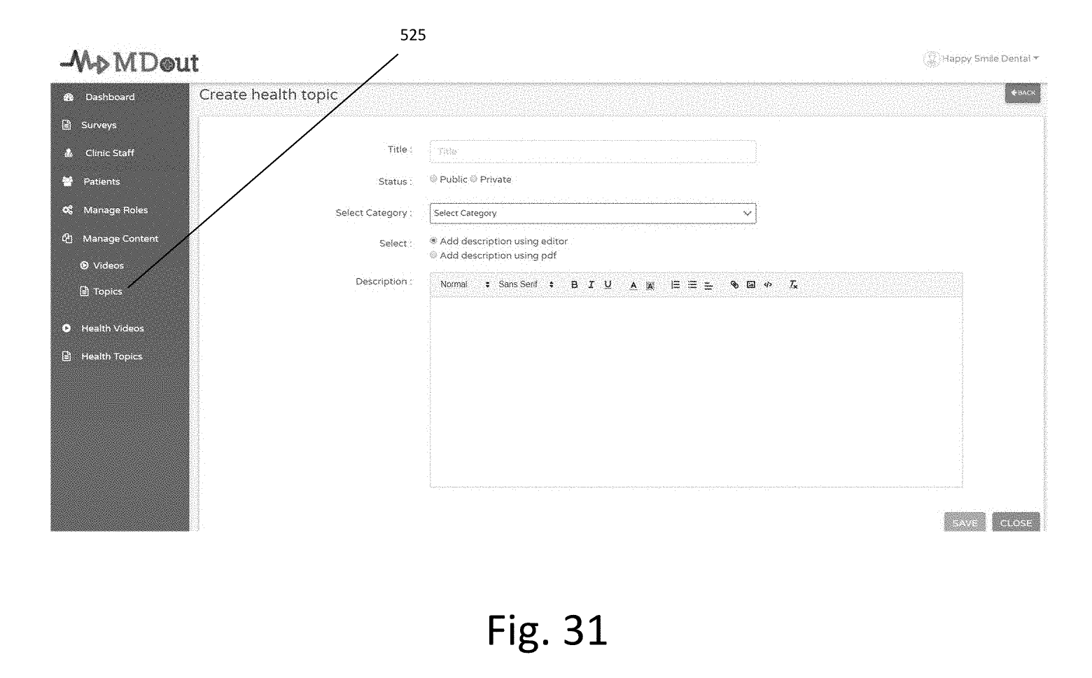

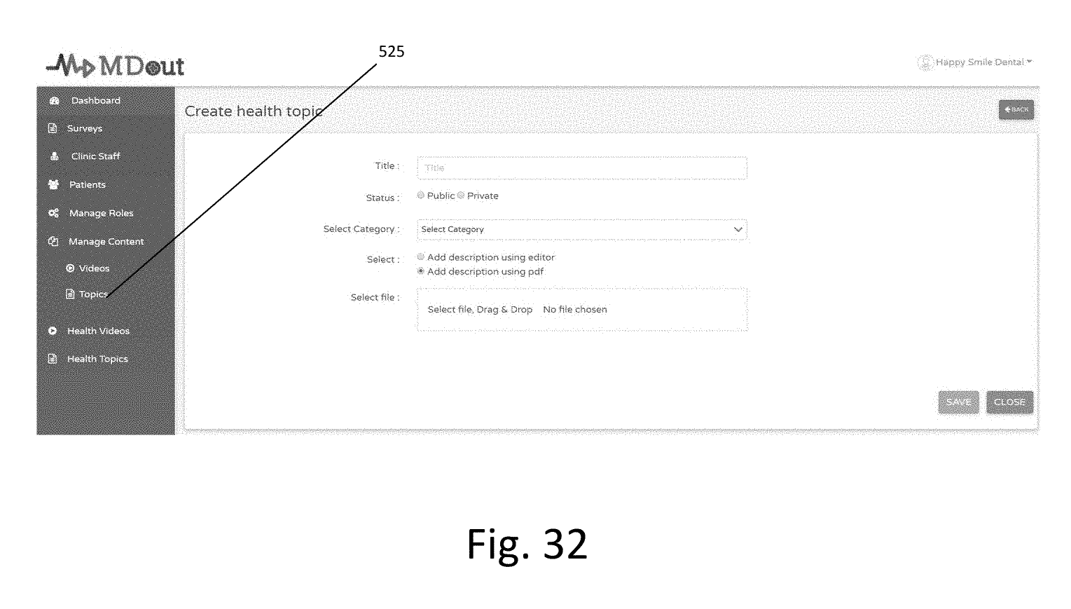

The page enables the clinic's administrator to create a title for the topic and to select whether the topic is public or private. The clinic's administrator can also select a category in which to place the topic. The page enables the user to further select whether to “add description using editor” or “add description using pdf,” as will be seen in FIGS. 31-32.

In some embodiments of the present invention, each time a private topic is added by the clinic's administrator, the server sends a message to all patients associated with the clinic that a new topic has been added.

FIG. 31 is a screenshot of the page after the clinic's administrator has chosen the option to “add description using editor” in FIG. 30. Interaction with the “add description using editor” option causes the server to display a “description” box which allows the user to type in a description for the topic.

FIG. 32 is a screenshot of the page after the clinic's administrator has chosen the option to “add description using pdf” in FIG. 30. Interaction with the “add description using pdf” option causes the server to display a “select file” category, with an option to select a file or to drag and drop a file into the resulting box.

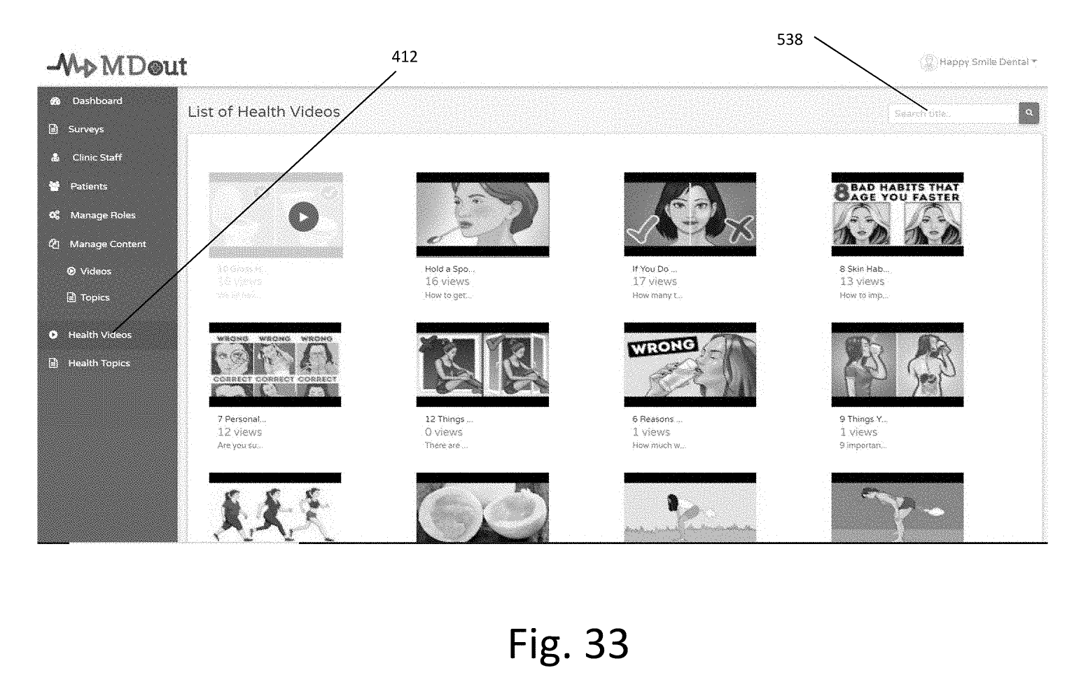

FIG. 33 is a screenshot of a list of health videos. The list is displayed when the clinic's administrator interacts with the “health videos” link 412 in the dashboard window.

Each video is represented by a picture from the respective video. Other information, such as at least the beginning of the title of the video, the number of views, and the beginning of the description of the video is included with each picture.

A “search” box 538 enables the user to search for a specific video via its title.

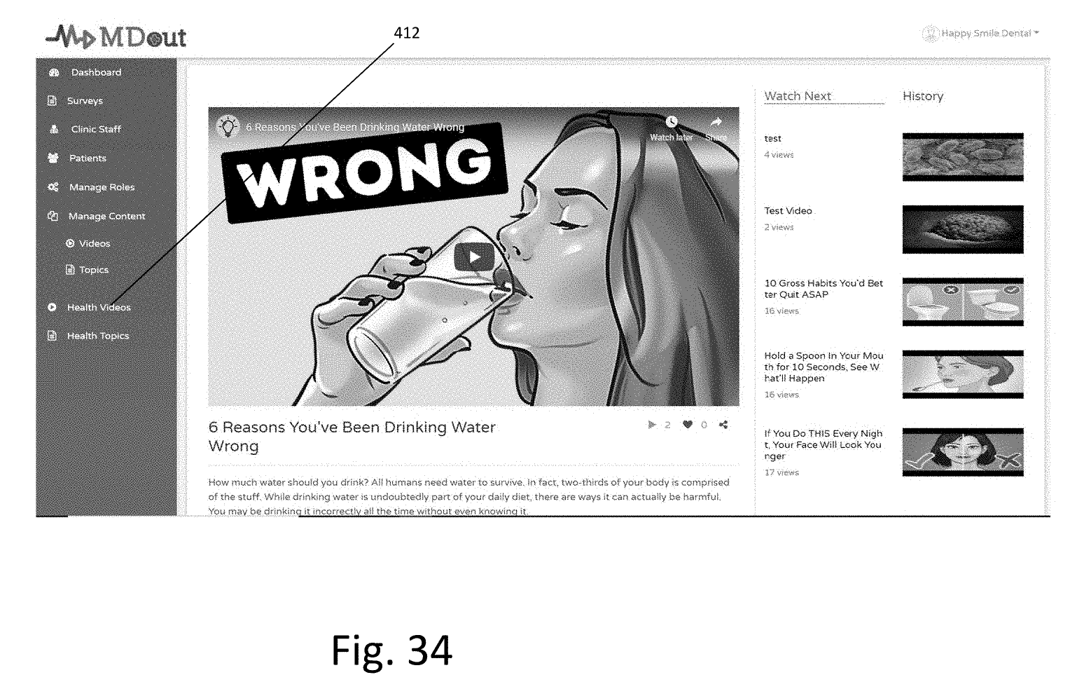

FIG. 34 is a screenshot of a video. Interaction with one of the videos in FIG. 33 causes the server to display the selected video.

The page also provides the entire description of the video below the video. The right side of the page provides a list of other videos under the “watch next” tab and a “history” tab for the user to view the history of videos watched.

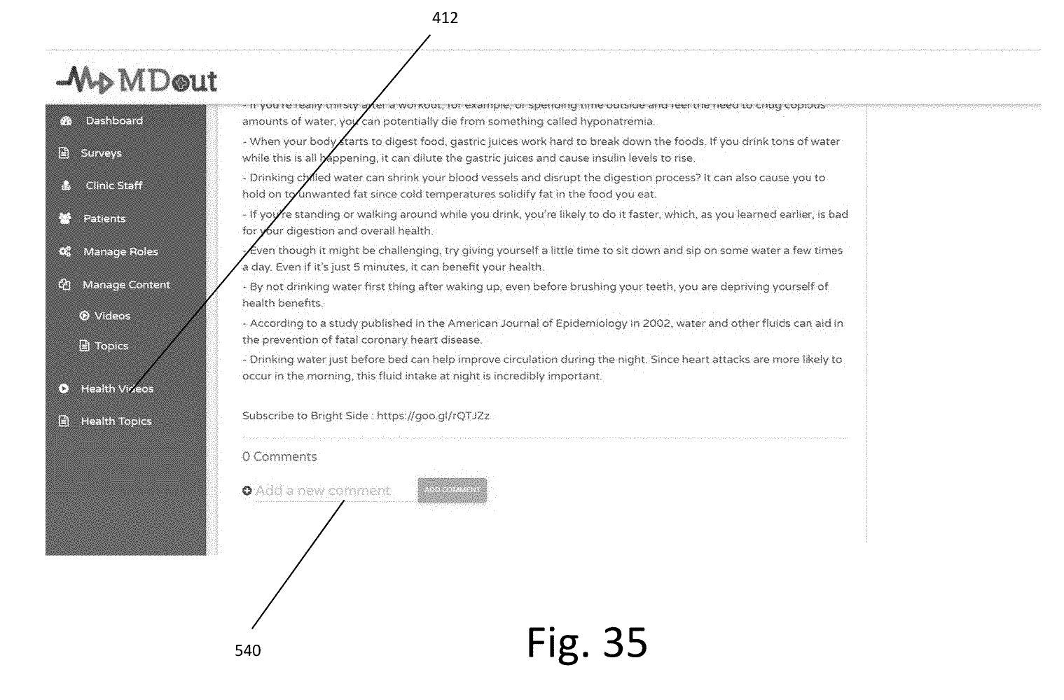

FIG. 35 is a screenshot of the continued description under the selected video in FIG. 34. An “add comment” box 540 enables the user to type in a new comment to be posted under the selected video.

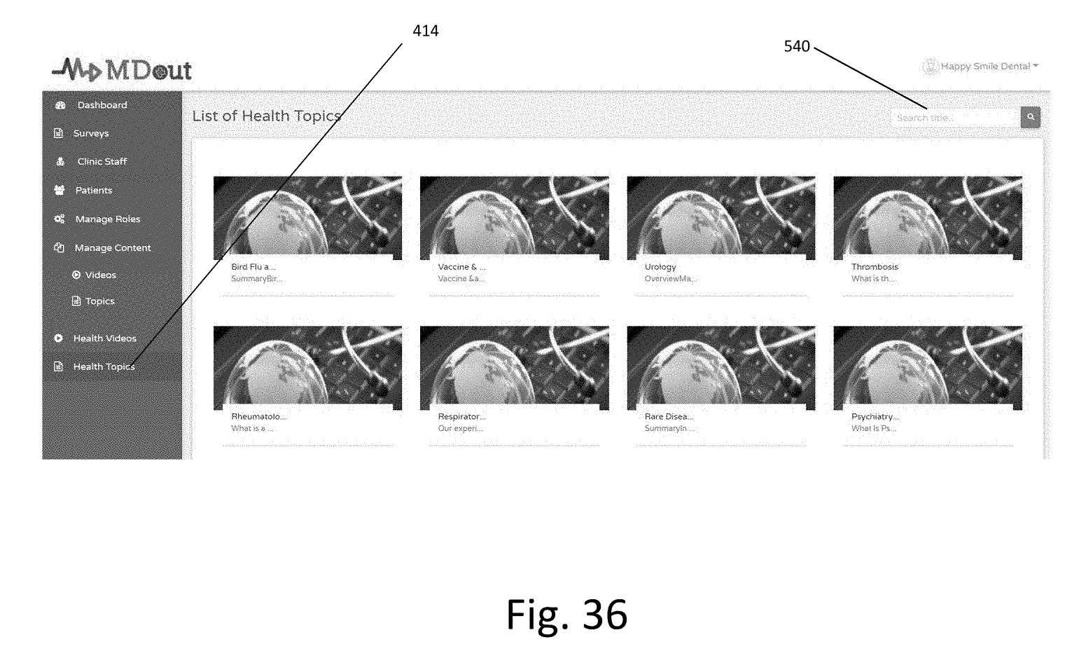

FIG. 36 is a screenshot of a list of health topics. The list is displayed when the clinic's administrator interacts with the “health topics” link 414 on the left side of the screen.

Each topic is represented by a picture and at least, a title and beginning of description. A “search” box 540 enables the user to search for a specific topic via its title.

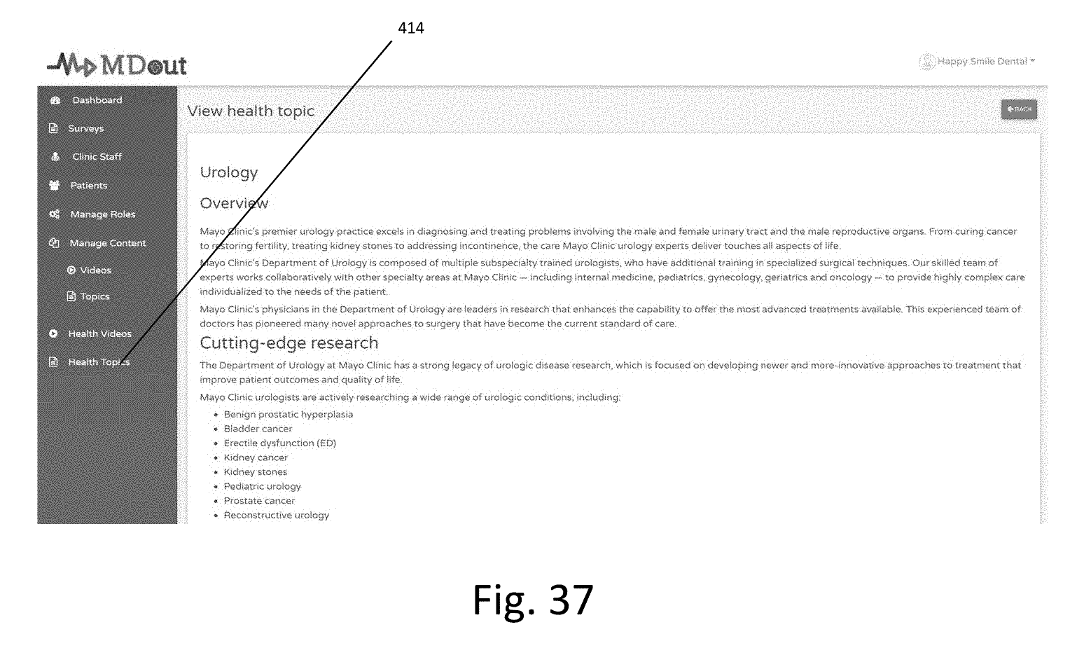

FIG. 37 is a screenshot of a selected health topic. Interaction with one of the topics in FIG. 36 causes the server to display the selected topic.

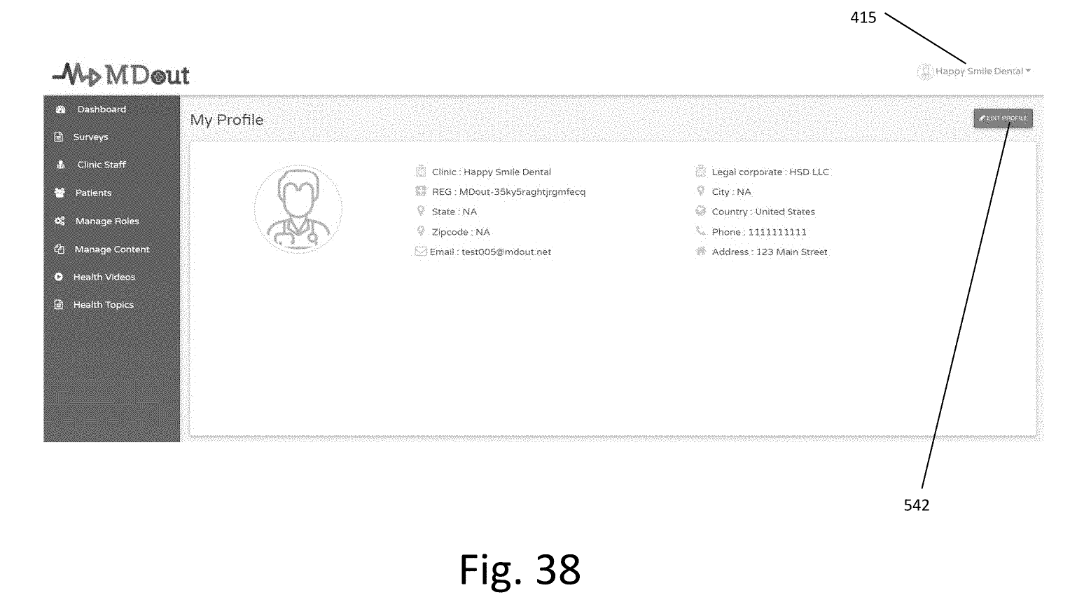

FIG. 38 is a screenshot of a clinic administrator's profile. Interaction with the “profile” option from the drop down menu of the clinic link 415 causes the server to display the administrator's profile. An “edit profile” option 542 enables the user to edit information such as at least clinic name, registration code, address, and email address.

FIGS. 39-45 are screenshots of images displayed by a communication device connected to the server of FIGS. 1 and 2 running the staff management tool, according to some embodiments of the present invention. The staff management tool is configured to enable a staff member to performed specific actions, as described below.

FIG. 39 is a screenshot of the dashboard of a provider user (staff member). Interaction with the “dashboard” 550 option on the left side of the screen causes the server to display the provider user's dashboard.

In some embodiments of the present invention, the dashboard screen includes one or more summary windows. Each window occupies a respective portion of the screen distinct from other windows. An assigned survey summary window 558 displays the number of surveys assigned to patients associated with the staff member. A survey completion summary window 560 displays the number of surveys that have been completed by patients associated with the staff member. A pending survey summary window 562 displays the number of surveys that have been assigned to patient associated with the staff member and have not been completed yet. Each summary window enables the staff to view more details by selecting the “view details” option under each summary window. The dashboard also includes the user's most viewed videos.

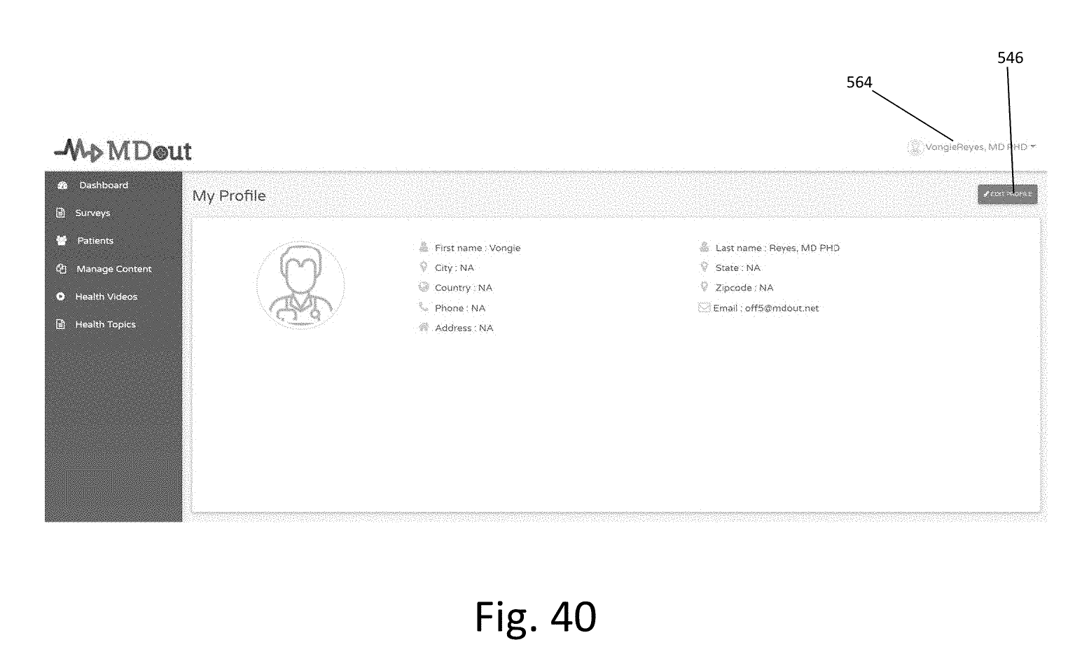

FIG. 40 is a screenshot of a provider user's profile. Interaction with the “profile” option from the drop down menu of the provider user link 564 causes the server to display the profile of the staff member. An “edit profile” option 546 enables the staff member to edit information such as at least the user name, phone number, address, and email address of the provider (i.e., staff member).

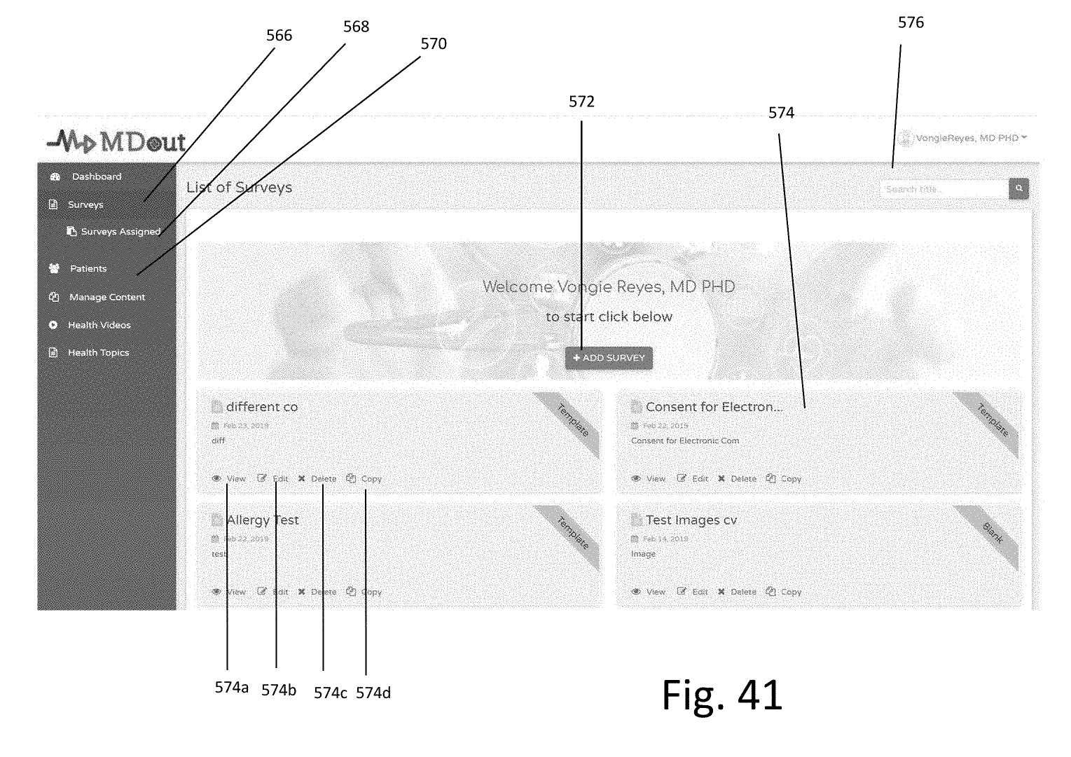

FIG. 41 is a screenshot of a survey page displayed to the medical staff member after the medical staff member has interacted with the “surveys” link 566. In the page of FIG. 41, a “surveys assigned” sub-link 568 appears under the “surveys” link 566. The sub-link 568 is optionally located between the “surveys” link 566 and the “patients” link 570.

The survey page includes an “add survey” button 572. Interacting with the “add survey” button enables the medical staff member to add a survey, in the same manner explained above for the clinic's administrator. The survey page also includes a “search” box 576 which enables the medical staff member to search for a survey via its title.

In some embodiments of the present invention, the survey page includes survey summary windows 574. Each survey summary window 574 is associated with a respective survey and includes a title of the survey, a short description of the survey, and at least three buttons: a view button 574a, and edit button 574b, and a delete button 574c. Optionally, the survey summary window 574 also includes a copy button 574d.

By interacting with the view button 574a in the survey summary window 574 associated with a desired survey, the medical staff member may view the survey as it would be viewed by a patient filling out the survey. By interacting with the edit button 574b, the medical staff member is directed to one or more pages with tools configured to enable the provider user to edit the survey, as shown in FIG. 13. By interacting with the delete button 574c in the survey summary window 574 associated with a desired survey, the medical staff member may delete the survey. By interacting with the copy button 574d in the survey summary window 574 associated with a desired survey, the medical staff member may create a new survey that is a copy of the desired survey.

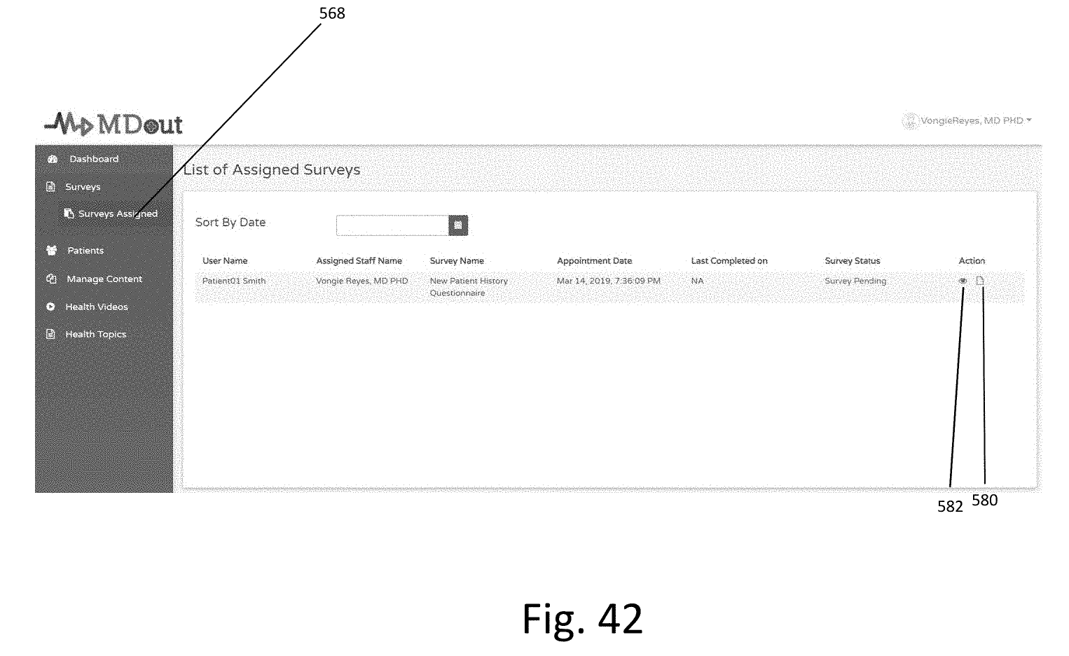

FIG. 42 is a screen shot of a list of surveys assigned to the medical staff member. Interacting with the “surveys assigned” sub-link 568 causes the server to display the list of surveys assigned to the patients assigned to respective medical staff member.

In some embodiments of the present invention, some data relating to each survey in the list is displayed near the survey. The data may include the user name of the patient, the name of the survey, the name of the medical staff member assigned to the patient, the date of the patient's appointment at the clinic, the date the survey was completed, and the status of the survey (completed or pending). Optionally, the provider user may view the survey by interacting with the eye icon 582, and add notes to the survey by interacting with the paper icon 580.

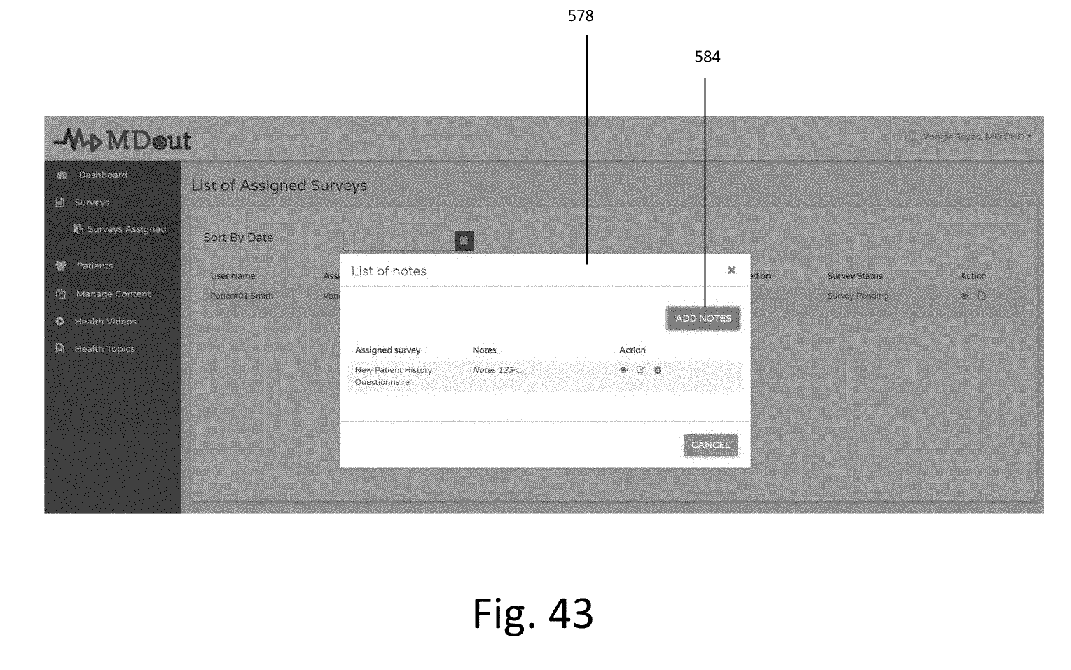

FIG. 43 is a screenshot of a prompt 578 that enables the medical staff member to view a list of notes for a particular survey. Interaction with the paper icon 580 of FIG. 42 causes the server to display this prompt 578. The prompt 578 may be displayed in the center of the screen, thereby reducing the list of assigned surveys to the background.

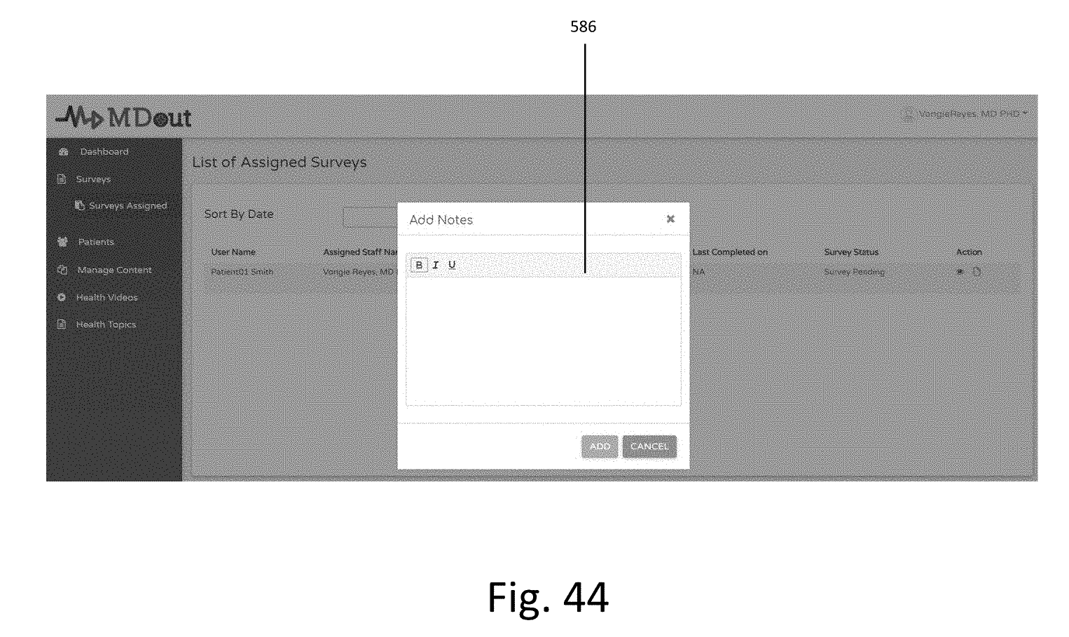

The prompt 578 displays a list of notes entered into the database for a particular survey. Within each note, the medical staff member may view, edit, or delete the note. Interacting with the eye icon enables the medical staff member to view the note, interacting with the pen and pad icon enables the medical staff member to edit the note, and interacting with the trash can enables the medical staff member to delete the note. An “add notes” button 584 enables the medical staff member to enter a note into the database for a specific survey. Interacting with the “add notes” button 584 causes the server to display a prompt 586 to create a note, as will be seen in FIG. 44.

FIG. 44 is a screenshot of a prompt window 586 that enables the provider user to create a note for a particular survey. Interaction with the “add notes” button 584 of FIG. 43 causes the server to display this prompt window 586.

The prompt window 586 comprises a text box for the provider user to enter a note into the database for the survey. At the least, the prompt window 586 enables the provider user to select a bold font, italic font, and underlined font. If a medical staff member wishes to edit an existing note by interacting with the pen and pad icon of FIG. 43, a prompt window similar to the prompt window 586 is displayed, with the text of the note in the text box.

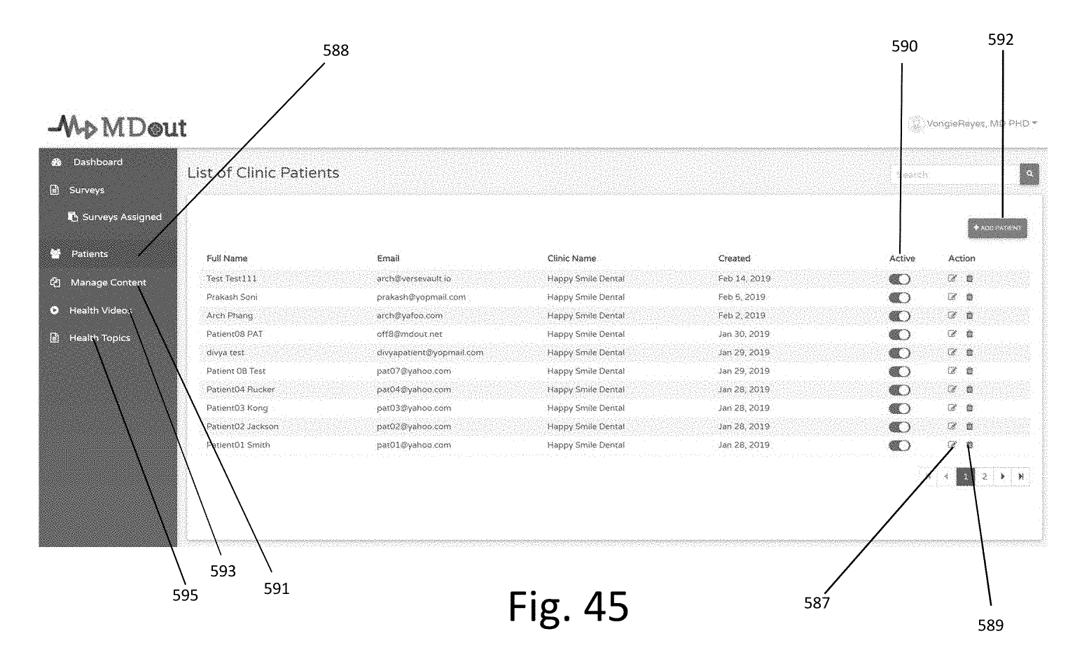

FIG. 45 is a screenshot of a list of patients assigned to the medical staff member. The list is displayed when the medical staff member user interacts with the “patients” link 588 on the left side of the screen.

A respective line is assigned to each patient. In the list, at least the name of each patient is listed along with other information, such as the email address of the patients, the clinic name, and the date in which the staff members were entered into the system (created).

In some embodiments of the present invention, an activity icon 590 may be interacted with to designate the patient as active or passive. The activity icon has two configurations: an “on” configuration indicating that the patient is active and an “off” configuration indicating that the patient is passive. Interacting with the activity icon switches the icon from one configuration to the other. In some embodiments of the present invention, the medical staff member may edit a patient's information or delete a patient from the database, as explained above with reference to the clinic's administrator by interaction with the appropriate “edit” icon 587 and the “delete” icon 589 associated with the desired patient.

An “add patient” button 592 is also displayed. Interaction with the “add patient” button 592 causes the server to display one or more pages with prompts that enable the medical staff member to enter a patient in the patient database, as explained above referring to the same action by the clinic's administrator.

It should be noted that, as described for the clinic's administrator above, the medical staff member may also manage content (by interacting with the “manage content” link 591), view videos (by interacting with the “health videos” link 593), and view articles (by interacting with the “health topics” link 595).

FIGS. 46-51 are screenshots of images displayed by a communication device connected to the server of FIGS. 1 and 2 running the patient management tool, according to some embodiments of the present invention.

FIG. 46 is a screenshot of the dashboard screen of a patient, which is displayed to the patient when the patient logs in. Also, interaction with the “dashboard” 594 option on the left side of the screen at any time causes the server to display the patient's dashboard screen.

In some embodiments of the present invention, the dashboard screen includes one or more summary windows. Each window occupies a respective portion of the screen distinct from other windows. An assigned survey summary window 596 displays the number of surveys assigned to the patient. A survey completion summary window 598 displays the number of surveys that have been completed by the patient. A pending survey summary window 600 displays the number of surveys that have been assigned to the patient and have not been completed yet. Each summary window enables the patient user to view more details by selecting the “view details” option under each summary window. The dashboard page also includes a patient link 602, which will be described below.

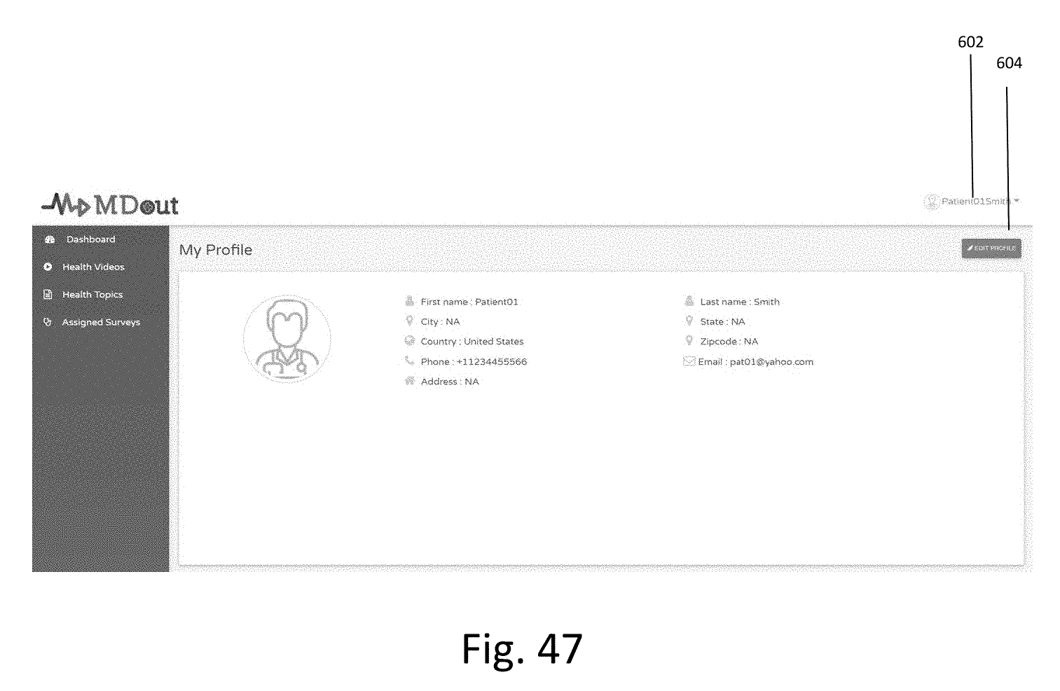

FIG. 47 is a screenshot of a patient's profile. Interaction with the “profile” option from the drop down menu of the patient link 602 causes the server to display the profile of the patient. An “edit profile” option 604 enables the patient to edit the patient's information, such as at least the user's name, phone number, address, and email address.

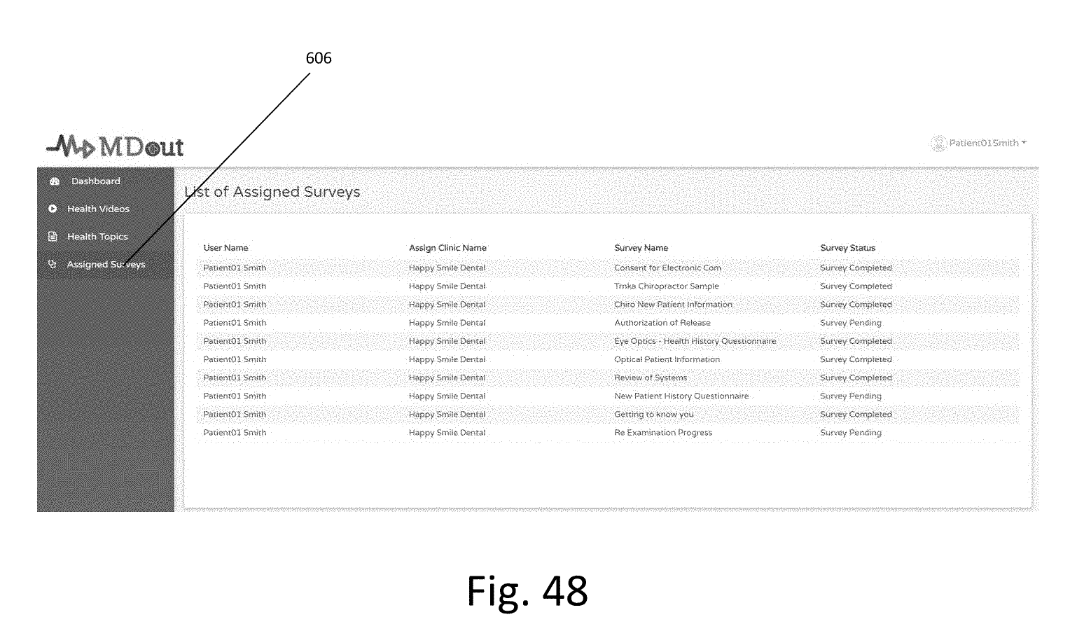

FIG. 48 is a screen shot of a list of surveys assigned to the patient user. Interacting with the “assigned surveys” link 606 on the left side of the screen causes the server to display the list of surveys assigned to the respective patient user.

In some embodiments of the present invention, some data relating to each survey in the list is displayed near the survey. The data may include the user name of the patient, the name of the survey, the name of the assigned clinic, and the status of the survey (completed or pending).

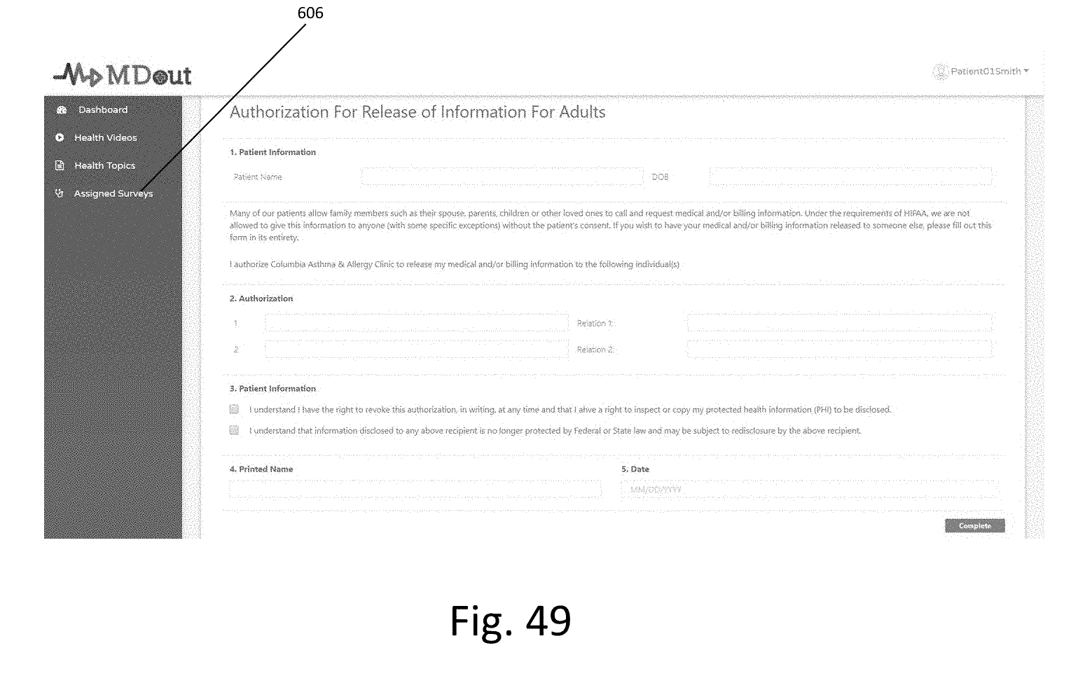

FIG. 49 is a screenshot of a survey assigned to a patient. Interacting with one of the surveys listed in FIG. 48 causes the server to display the survey.

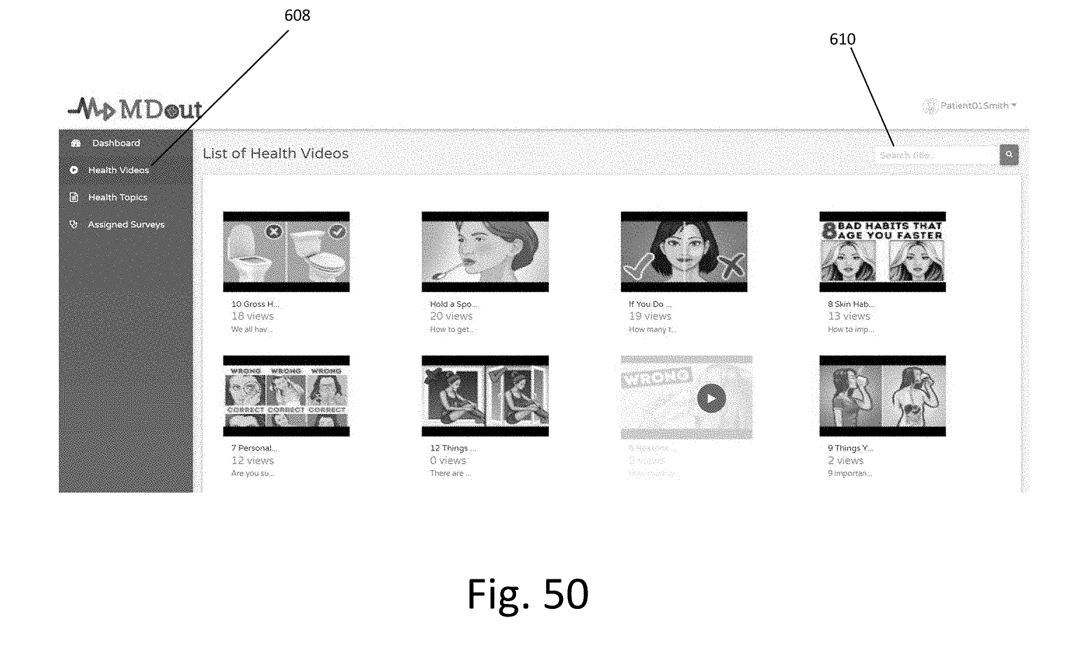

FIG. 50 is a screenshot of a list of health videos. The list is displayed when the patient interacts with the “health videos” link 608 in the dashboard window. The health videos are videos posted by the clinic's administrator of a clinic associated with the patient, a staff member of a clinic associated with the patient, or a support administrator.

Each video is represented by a picture from the respective video. Other information, such as at least the beginning of the title of the video, the number of views, and the beginning of the description of the video is included with each picture. By selecting a video, the patient may view a video, as described above with reference to the clinic's administrator. In some embodiments of the present invention, the patient may also post a comment about the video, as described above with reference to the clinic's administrator.

A “search” box 610 enables the patient user to search for a specific video via its title.

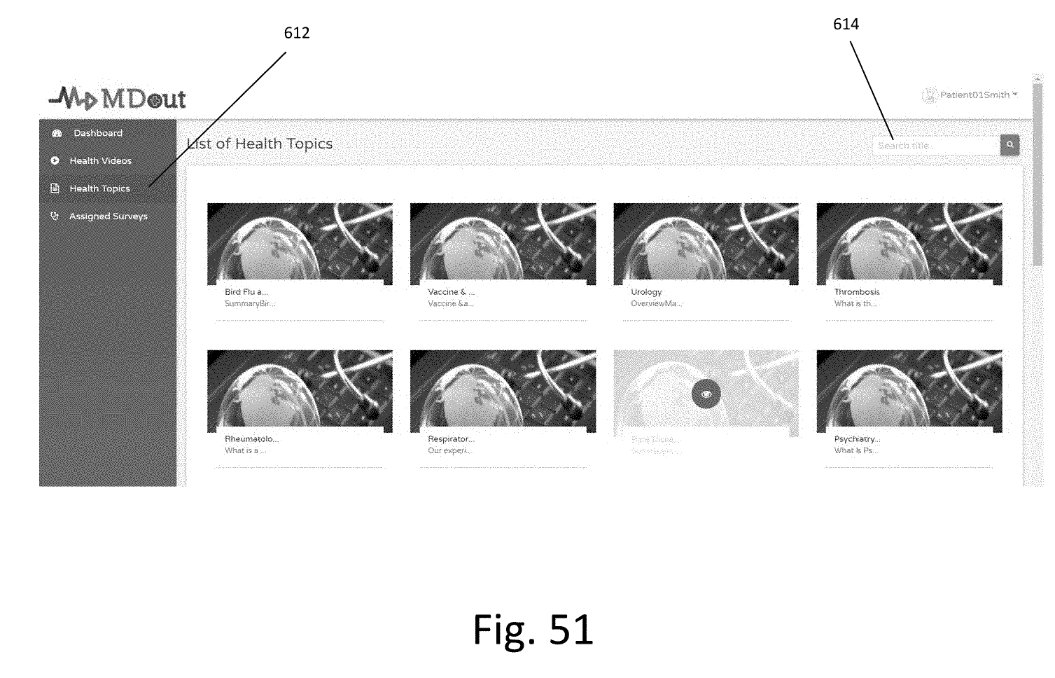

FIG. 51 is a screenshot of a list of health topics (articles). The list is displayed when the patient user interacts with the “health topics” link 612 on the left side of the screen. The health topics are articles posted by the clinic's administrator of a clinic associated with the patient, a staff member of a clinic associated with the patient, or a support administrator.

Each topic is represented by a picture and at least, a title and beginning of description. By selecting an article from the list, the patient may view the selected article, as described above with reference to the clinic's administrator. In some embodiments of the present invention, the patient may also post a comment about the video, as described above with reference to the clinic's administrator. A “search” box 614 enables the patient user to search for a specific topic via its title.

FIGS. 52-70 are screenshots of images by a communication device connected to the server of FIGS. 1 and 2 running the system management tool, according to some embodiments of the present invention.

FIG. 52 is a screenshot of a support administrator's dashboard screen which is displayed to the patient when the patient logs in. Also interacting with the “dashboard” link 616 on the left side of the screen causes the server to display the dashboard.

In some embodiments of the present invention, the dashboard screen includes one or more summary windows. Each window occupies a respective portion of the screen distinct from other windows. A survey summary window 618 displays the number of surveys available. A first clinic summary window 620 displays the number of clinics on the clinic database. A patient summary window 622 displays the number of patients on the patient database. A second clinic summary window 624 displays a list of clinics registered in the system. A content access summary window 626 displays a list of content (videos and articles) created in the content database.

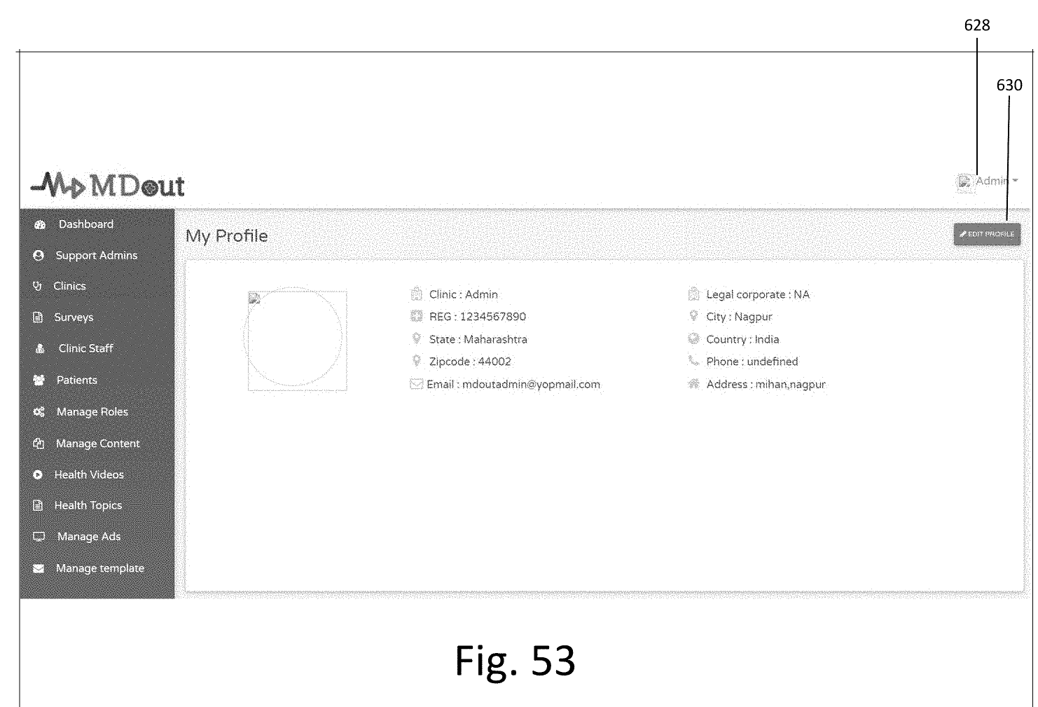

FIG. 53 is a screenshot of a support administrator's profile. Interacting with the “profile” option from the drop down menu of the support administrator link 628 causes the server to display the profile of the support administrator. An “edit profile” option 630 enables the support administrator to edit information such as the support administrator's name, phone number, address, and email address.

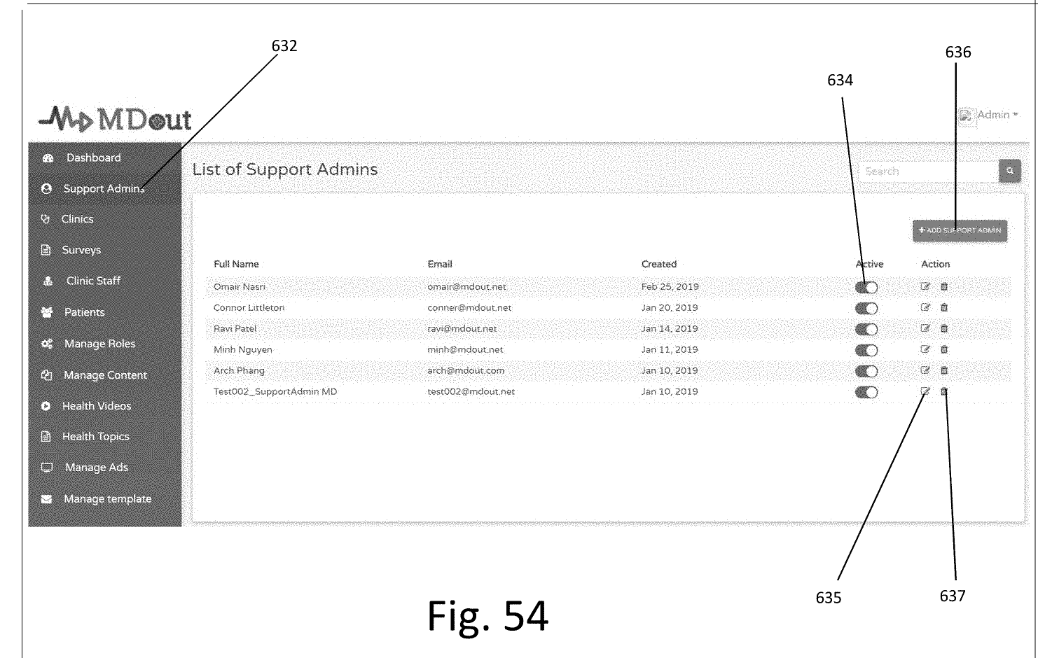

FIG. 54 is a screenshot of a list of support administrators. The list is displayed when the support administrator interacts with the “support admins” link 632 on the left side of the screen.

A respective line is assigned to each support administrator. In the list, at least the name of each administrator is listed along with other information, such as the email address and the date in which the administrators were entered into the system (created).

In some embodiments of the present invention, an activity icon 634 may be interacted with to designate the administrator as active or passive. The activity icon has two configurations: an “on” configuration indicating that the administrator is active and an “off” configuration indicating that the administrator is passive. Interacting with the activity icon switches the icon from one configuration to the other.

Interaction with an “edit” button and 635 a “delete” button 637 enable the support administrator to respectively edit information relating to corresponding support administrator and delete a support administrator from the support administrator database. This is done in a manner similar to editing and deleting patient data, as explained above with reference to clinic administrators.

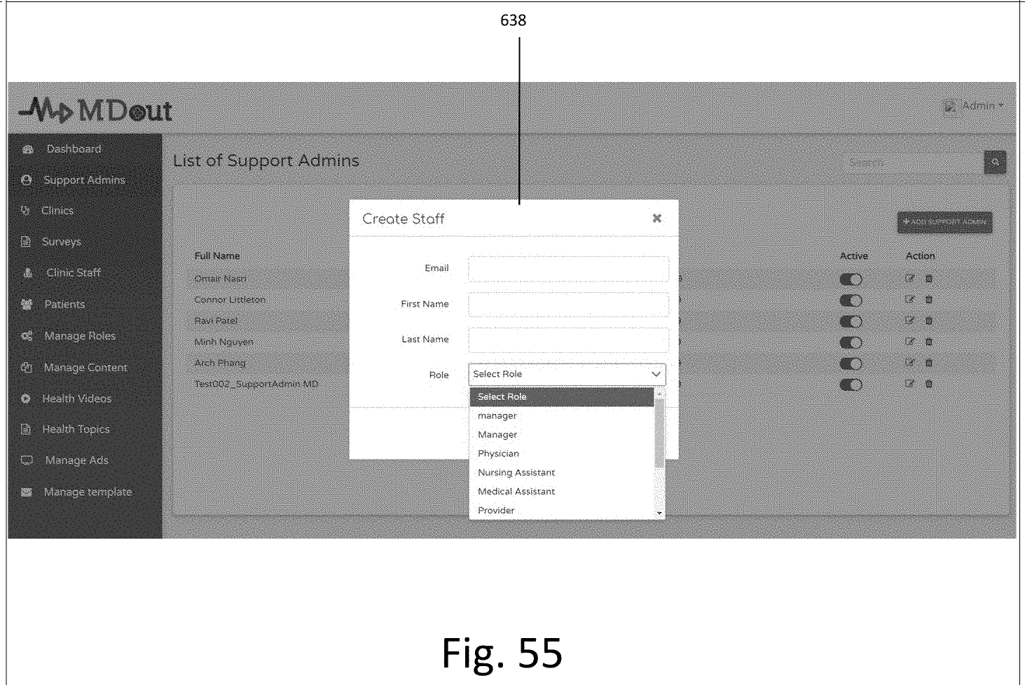

An “add support admin” button 636 is also displayed. Interacting with the “add support admin” button 636 causes the server to display one or more pages with prompts that enable the user to enter a support administrator in the support administrator database, which will be seen in FIG. 55.

FIG. 55 is a screenshot of a prompt window 638 that enables the support administrator to create a new support administrator in the database. The prompt window 638 may be displayed in the center of the screen, thereby reducing the list of support administrators to the background.

The prompt 638 for creating a support administrator allows the support administrator to enter the new administrator's email address, first name, last name, and role. Interaction with the prompt window 638 functions in the same manner as the prompt window 490 of FIG. 16.

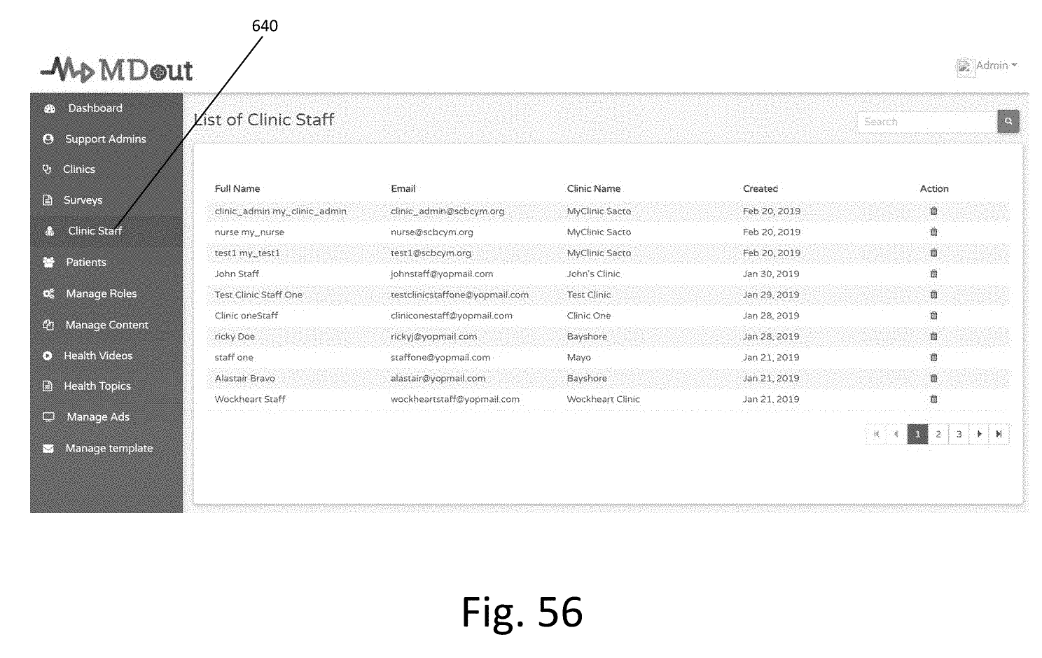

FIG. 56 is a screenshot of a list of clinic staff. The list is displayed when the support administrator interacts with the “clinic staff” link 640 on the left side of the screen.

A respective line is assigned to each staff member. In the list, at least the name of each staff member is listed along with other information, such as the email address, clinic name, and the date in which the clinic staff members were entered into the system (created).

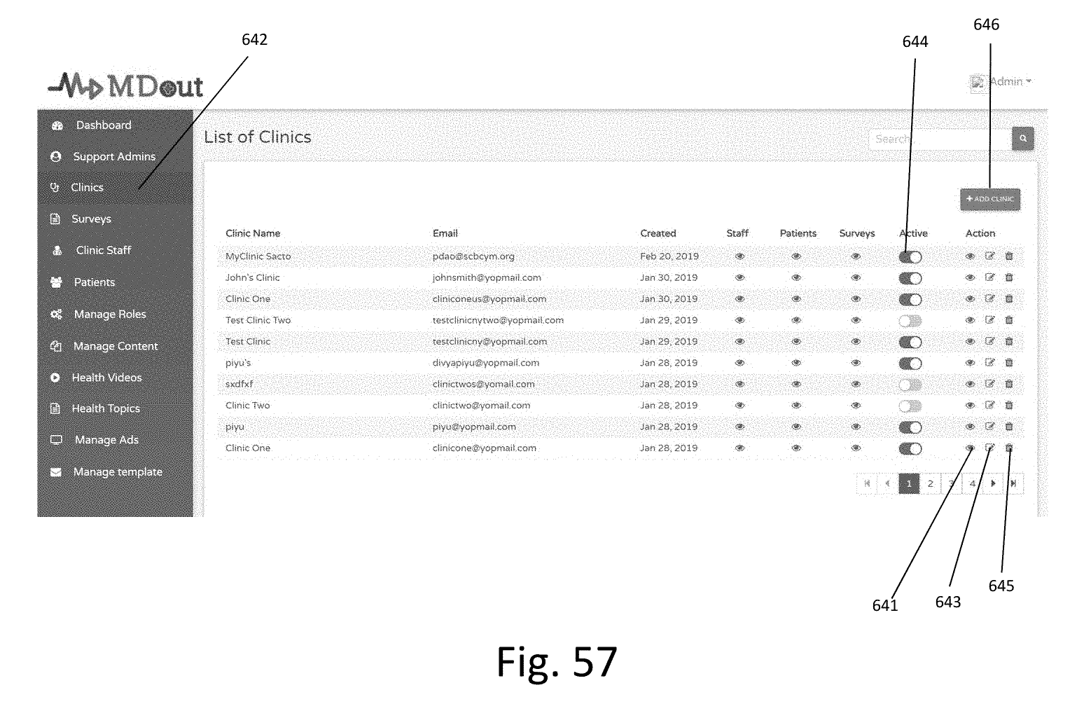

FIG. 57 is a screenshot of a list of clinics. The list is displayed when the support administrator interacts with the “clinics” link 642 on the left side of the screen.

A respective line is assigned to each clinic. In the list, at least the name of each clinic is listed along with other information, such as the email address of the clinic's administrator, and the date in which the clinics were entered into the system (created). The page also enables the support administrator to view the staff members, patients, and surveys for each respective clinic in the database.

In some embodiments of the present invention, an activity icon 644 may be interacted with to designate the clinic as active or passive. The activity icon has two configurations: an “on” configuration indicating that the clinic is active and an “off” configuration indicating that the clinic is passive. Interacting with the activity icon switches the icon from one configuration to the other.

An “add clinic” button 646 is also displayed. Interacting with the “add clinic” button 646 causes the server to display one or more pages with prompts that enable the user to enter a clinic in the database.

The page of FIG. 57 also includes a “view” button 641, an “edit” button 643, and a “delete” button 645 associated with each clinic. The “view” button 641 allows the support administrator to view information of a clinic. The “edit” 643 button allows the support administrator to edit information of a clinic. The “delete” 645 button allows the support administrator to delete information of a clinic from the clinic database. This is done in a manner similar viewing, editing, and deleting patients in FIG. 17.

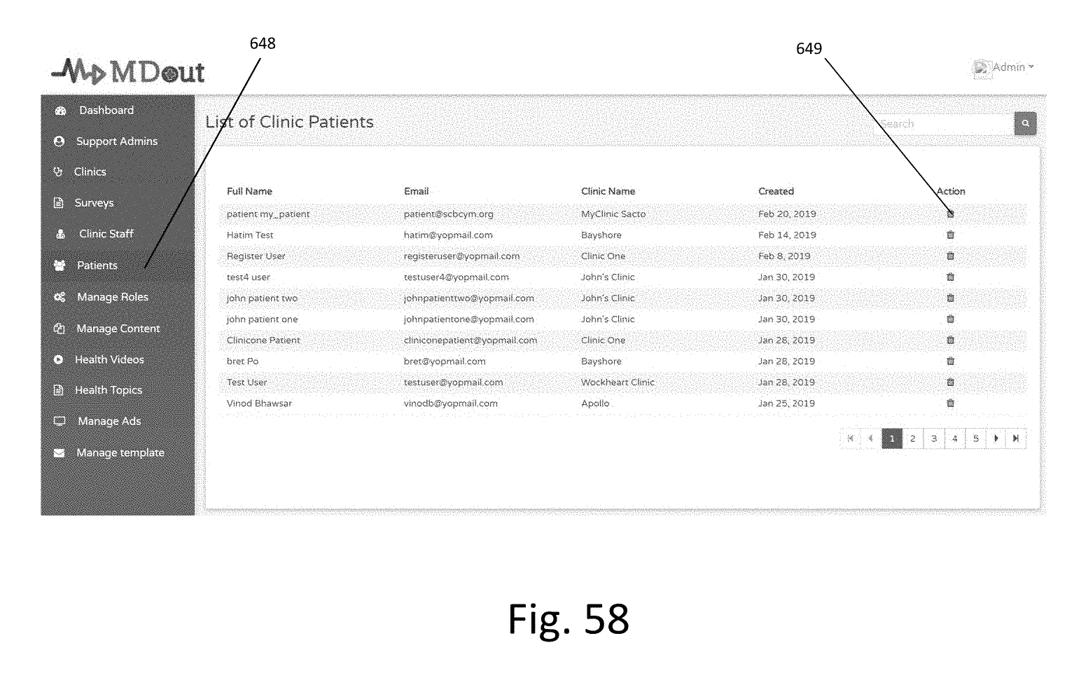

FIG. 58 is a screenshot of a list of clinic patients in the patient database. The list is displayed when the support administrator interacts with the “patients” link 648 on the left side of the screen.

A respective line is assigned to each patient. In the list, at least the name of each patient is listed along with other information, such as the email address, clinic name, and the date in which the patients were entered into the system (created). In some embodiments of the present invention, the support administrator can delete a patient from the patient database by interacting with a “delete” button 649 associated with the patient.

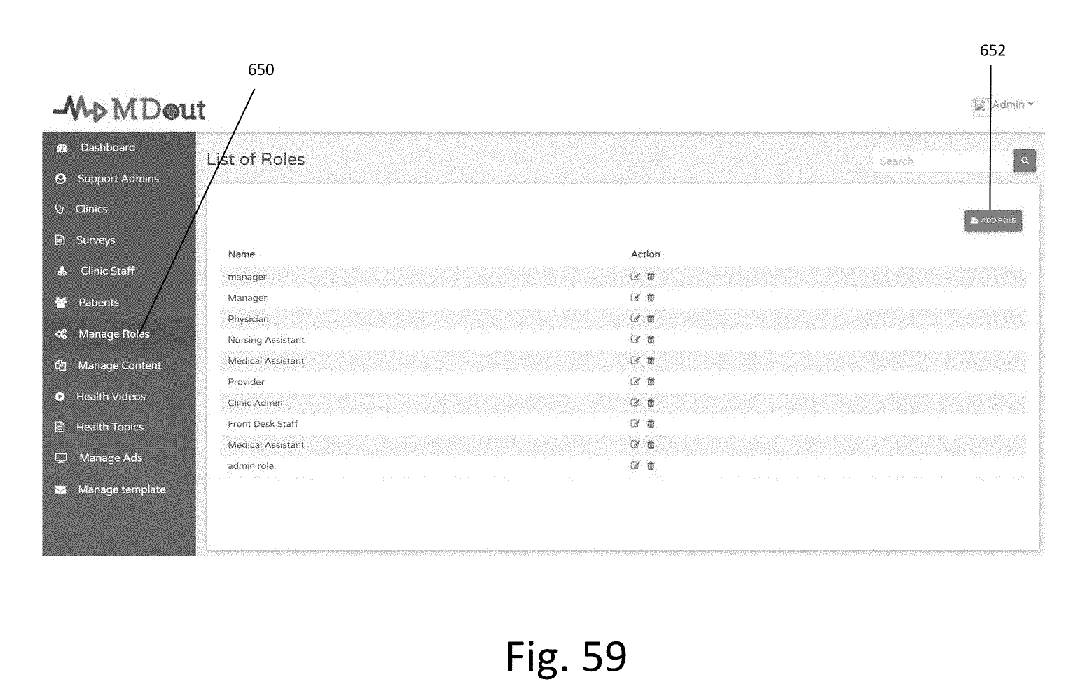

FIG. 59 is a screenshot of a list of roles of support administrators and clinic staff members. Once a new staff member role is created, the new staff member role is available to all clinic administrators of all clinics. The list is displayed when the support administrator interacts with the “manage roles” link 650 on the left side of the screen.

A respective line is assigned to each role. In the list, at least the name of each role is listed. The support administrator also has the ability to edit and delete roles using the pen and pad and trash can icons, respectively. These actions are performed in a manner similar for editing and deleting staff member roles by a clinic's administrator, as explained above.

An “add role” button 652 is also displayed. Interacting with the “add role” button 652 causes the server to display one or more pages with prompts that enable the support administrator to enter a role in the database. These actions are performed in a manner similar for creating staff member roles by a clinic's administrator, as explained above.

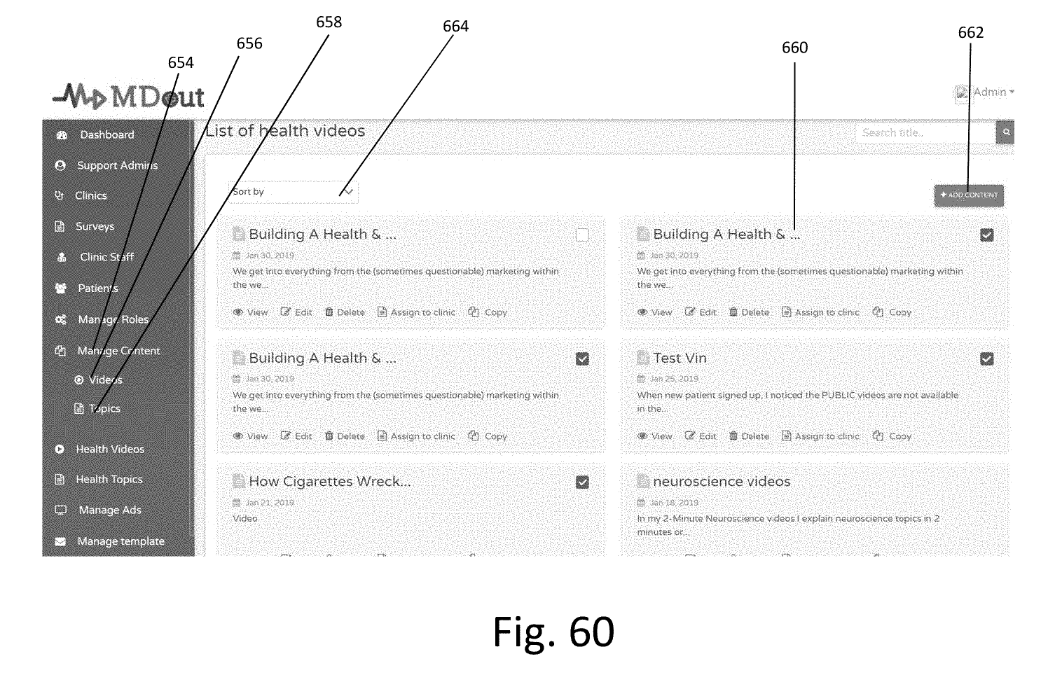

FIG. 60 is a screenshot of a list of health videos. When the “manage content” link 654 on the left side of the screen is interacted with, a “videos” sub-link 656 and “topics” sub-link 658 are displayed under the “manage content” link 654. The list of health videos is displayed when the support administrator interacts with the “videos” sub-link 656.

A respective video summary window 660 is assigned to each video. In the video summary window 660, at least the name of each video is listed along with icons such as “view,” “edit,” “delete,” “assign to clinic,” and “copy.” Other information may be displayed, such as the date in which the video was entered into the system (created) and the beginning of the description of the video. Interaction with the “view” icon associated with a video enables the support administrator to view the video, as explained above with reference to clinic administrator. Interaction with the “edit” icon associated with a video enables the support administrator to edit properties associated the video, such as the name and description of the video. Interaction with the “delete” icon associated with a video causes the video to be deleted. Interaction with the “assign to clinic” icon associated with a video enables the support administrator to assign the video to one or more clinics, making the video visible to the administrator of the clinic, the staff of the clinic, and to the patients of the clinic.

An “add content” button 662 is also displayed. Interaction with the “add content” button 662 causes the server to display one or more pages that enable the support administrator to create a video in the database. Videos ma be created by the support administrator in the same way as videos are created by a clinic's administrator, as described above.

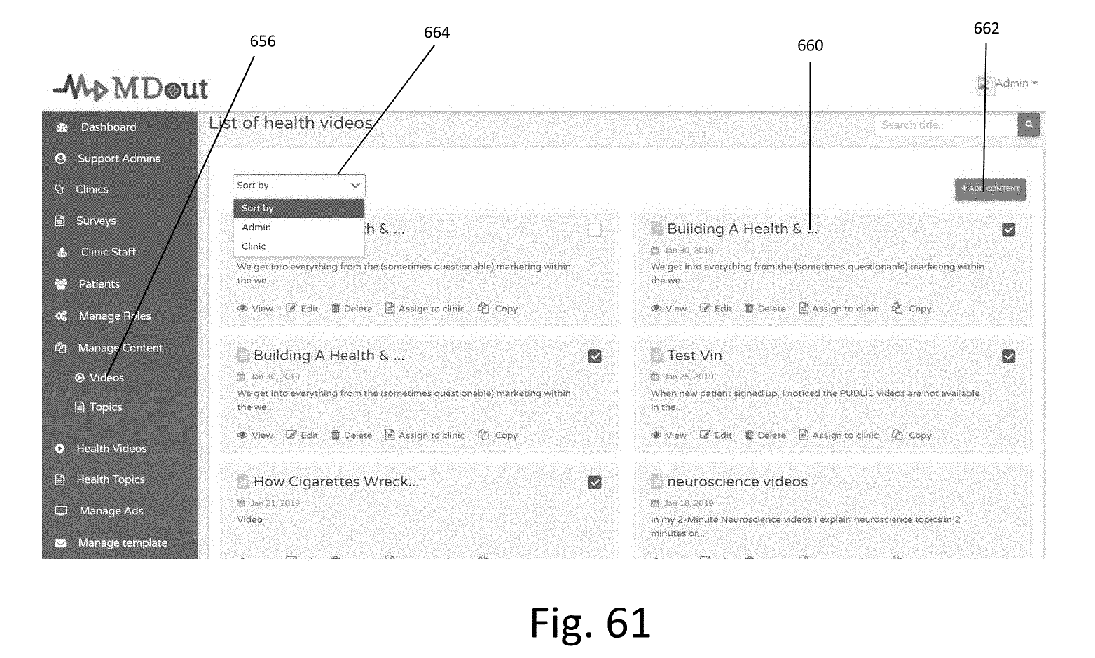

A filtering button 664 enables the support administrator to sort the videos via various filters such as “admin” and “clinic,” as will be seen in FIGS. 61-64.

FIG. 61 is a screenshot of the list of health videos. Interacting with the filtering button 664 causes the server to display a drop down list comprising “admin” and “clinic.”

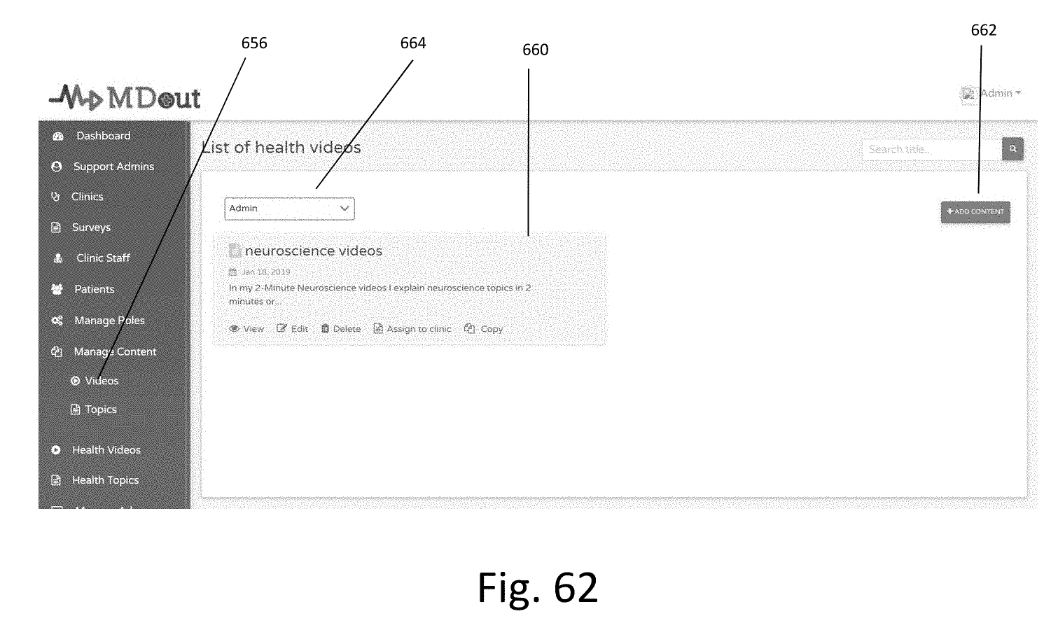

FIG. 62 is a screenshot of a list of health videos filtered via “admin.” Interacting with the “admin” option on the drop down list of the filtering button 664 causes the server to display the list of health videos created by the support administrator.

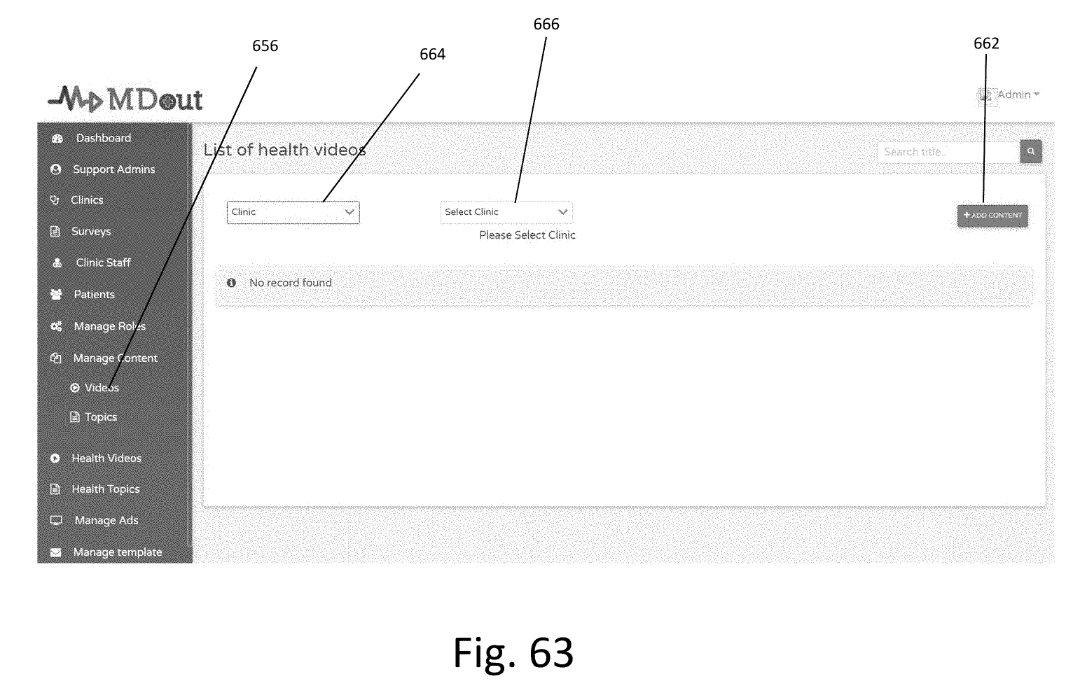

FIG. 63 is a screenshot of a list of health videos filtered via “clinic.” Interacting with the “clinic” option on the drop down list of the filtering button 664 causes the server to display a second button 666 enabling the support administrator to select a clinic name from a drop down list.

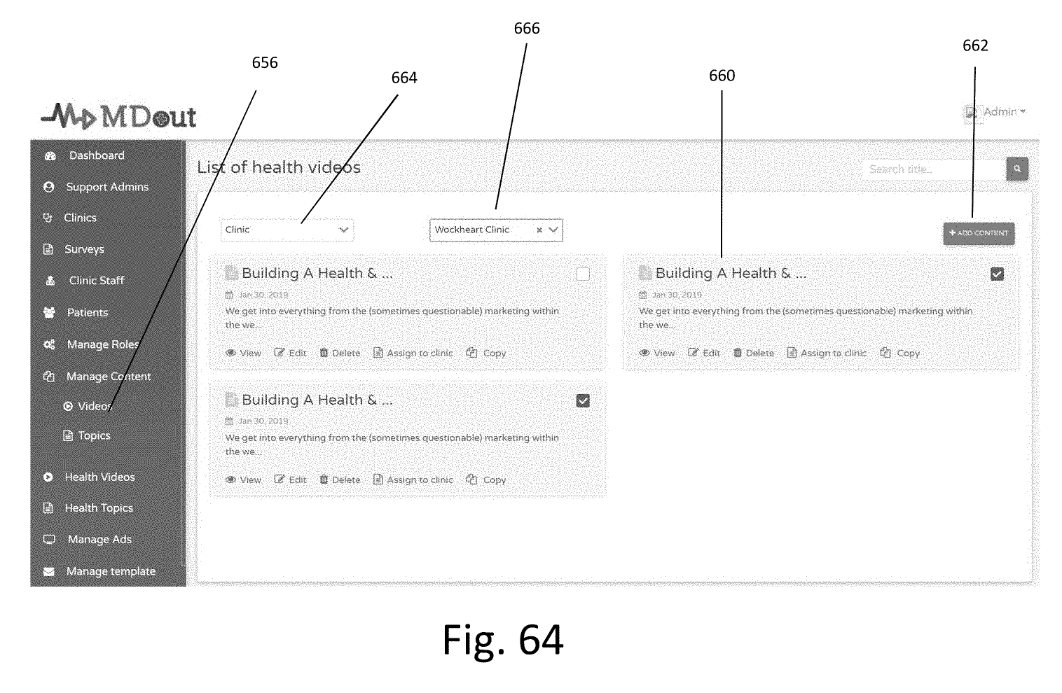

FIG. 64 is a screenshot of a list of health videos filtered via a selected clinic. Interacting with the second button 666 causes the server to display a list of videos associated with the content based on the type of clinic from the drop down list. The content will be specific to each particular clinic based on the category the clinic is associated themselves with. For example, an allergy clinic will see allergy content first or a dental clinic will see dental healthcare content first.

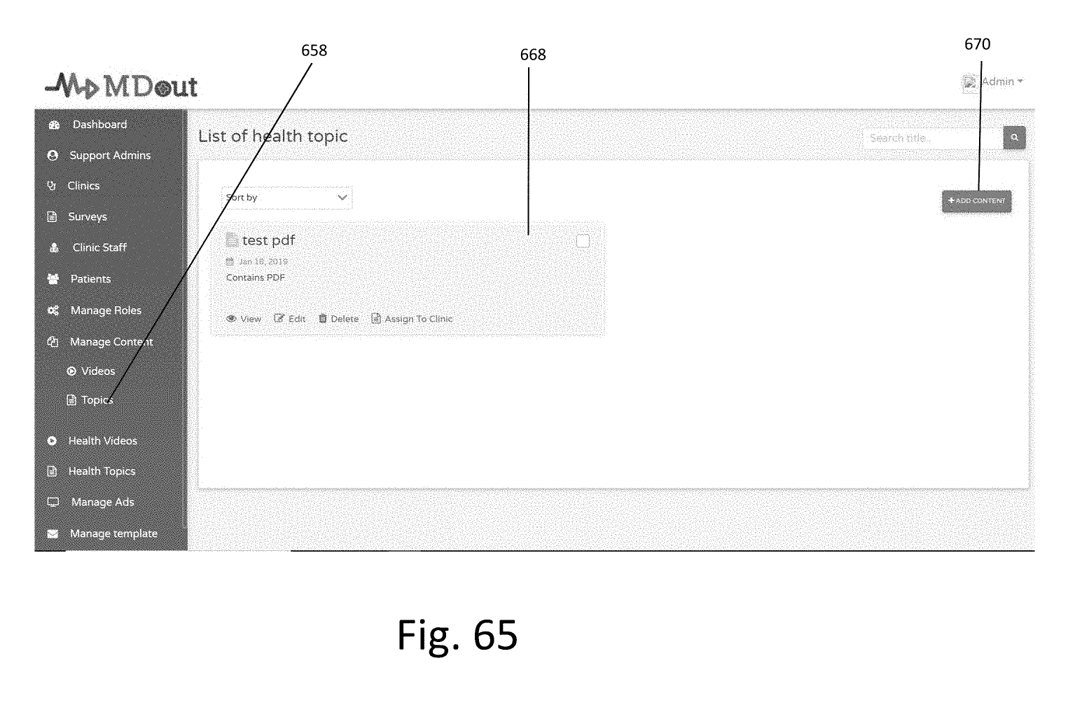

FIG. 65 is a screenshot of a list of health topics. The list of health topics/articles is displayed when the support administrator interacts with the “topics” sub-link 658 under the “manage content” link 654 on the left side of the screen.

A respective topic summary window 668 is assigned to each topic. In the topic summary window 668, at least the name of each video is listed along with icons such as “view,” “edit,” “delete,” and “assign to clinic.” Other information may be displayed, such as the date in which the video was entered into the system (created) and the beginning of the description of the topic. Interaction with the “view” icon associated with a topic enables the support administrator to view the topic, as explained above with reference to clinic administrator. Interaction with the “edit” icon associated with a topic enables the support administrator to edit properties associated the topic, such as the name and description of the video. Interaction with the “delete” icon associated with a topic causes the topic to be deleted. Interaction with the “assign to clinic” icon associated with a topic enables the support administrator to assign the topic to one or more clinics, making the topic visible to the administrator of the clinic, the staff of the clinic, and to the patients of the clinic.

An “add content” button 670 is also displayed. Interaction with the “add content” button 670 causes the server to display one or more pages that enable the support administrator to create a topic in the database in the same manner that a clinic's administrator creates a topic.

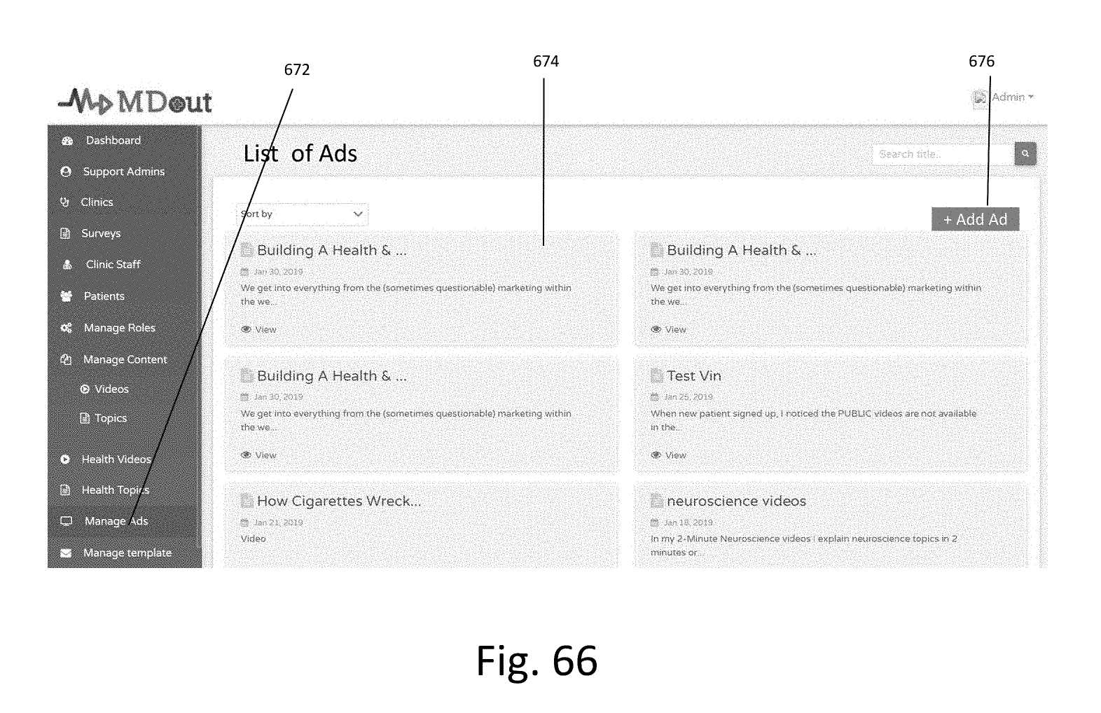

FIG. 66 is a screenshot of a list of ads. The list of ads is displayed when the support administrator interacts with the “manage ads” link 672 on the left side of the screen.

A respective ad summary window 674 is assigned to each ad. In the ad summary window 674, at least the name of each ad is listed along with a “view” icon. Other information may be displayed, such as the date in which the video was entered into the system (created) and the beginning of the description of the ad.

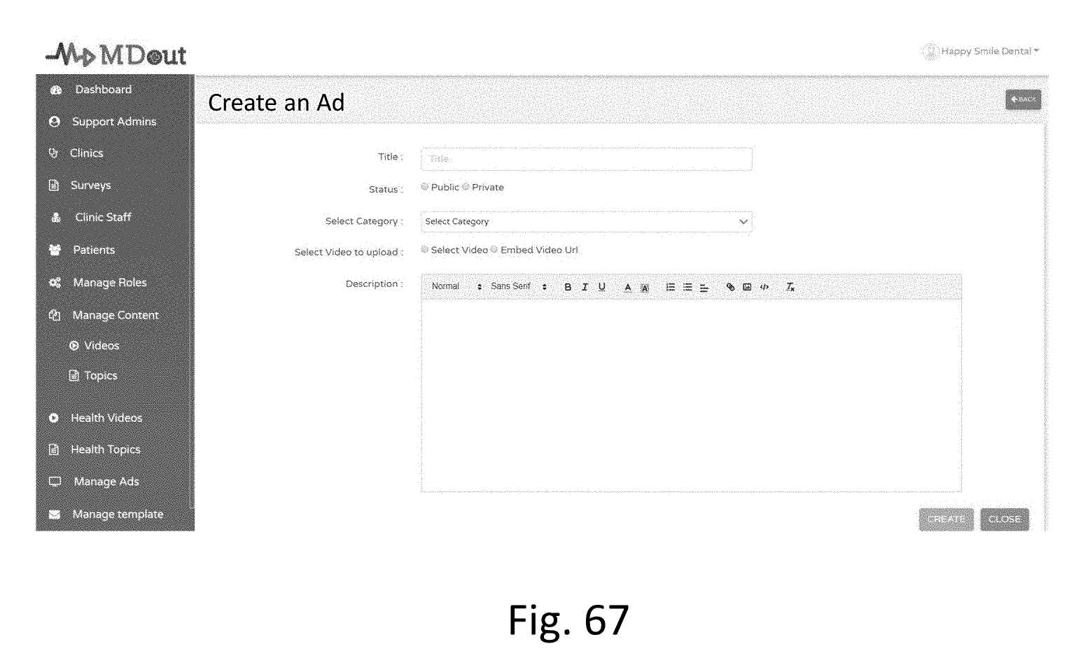

An “add ad” button 676 is also displayed. Interaction with the “add ad” button 676 causes the server to display one or more pages that enable the support administrator to create an ad in the database, which will be seen in FIG. 67.

FIG. 67 is a screenshot of a page that enables the support administrator to create an ad to add to the ad database. Interaction with the “add ad” button 676 of FIG. 66 causes the server to display this page.

The page enables the support administrator to create a title for the ad and to select whether the ad is public or private. A private ad is viewable by patients and staff of the clinic. A public video is meant to be viewable by all users registered in the server, and needs to be approved by a system's administrator. The support administrator may also select a category in which to place the ad and provide a description of the ad. The page enables the support administrator to further choose whether to “select video” (i.e. upload a video stored on the support administrator's device) or “embed video URL.”

Selecting the category of the ad associate the ad to videos and/or articles of the same category. The ad is shown to a patient when the patient views a video and/or reads an article having the same category as the video or article.

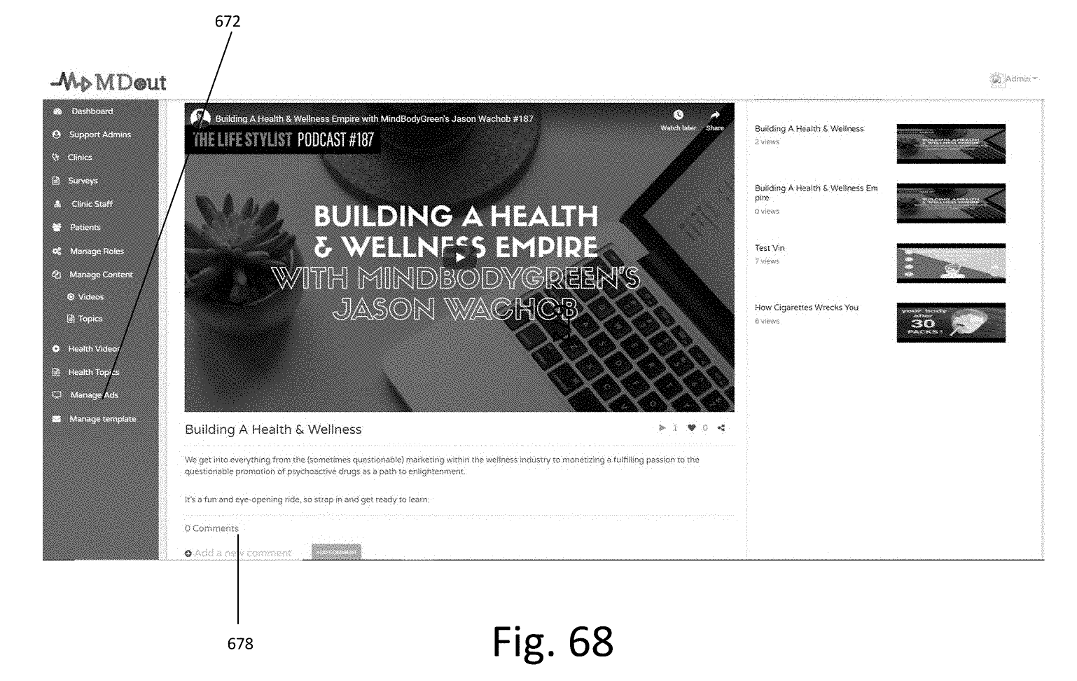

FIG. 68 is a screenshot of a selected ad. Interacting with an ad summary window 674 in FIG. 66 causes the server to display the selected ad. The page also enables the support administrator to view icons of other ads on a right side of the screen in list format. The page enables the support administrator to add a comment in an “add comment” box 678.

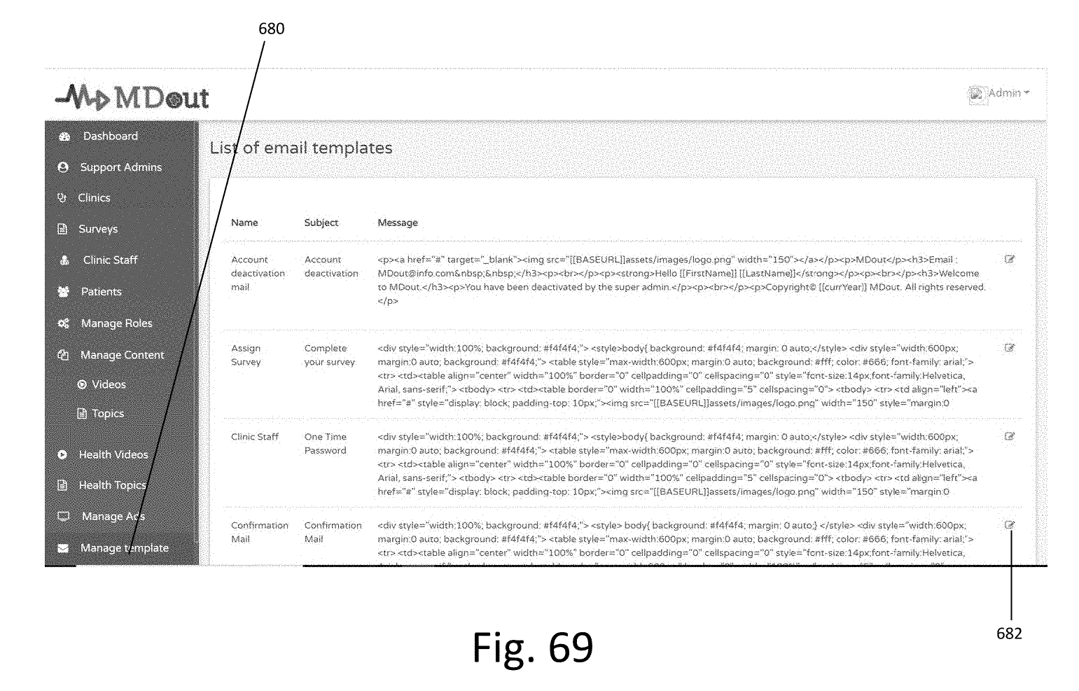

FIG. 69 is a screenshot of a list of email templates. The list of templates is displayed when the support administrator interacts with the “manage templates” link 680 on the left side of the screen.

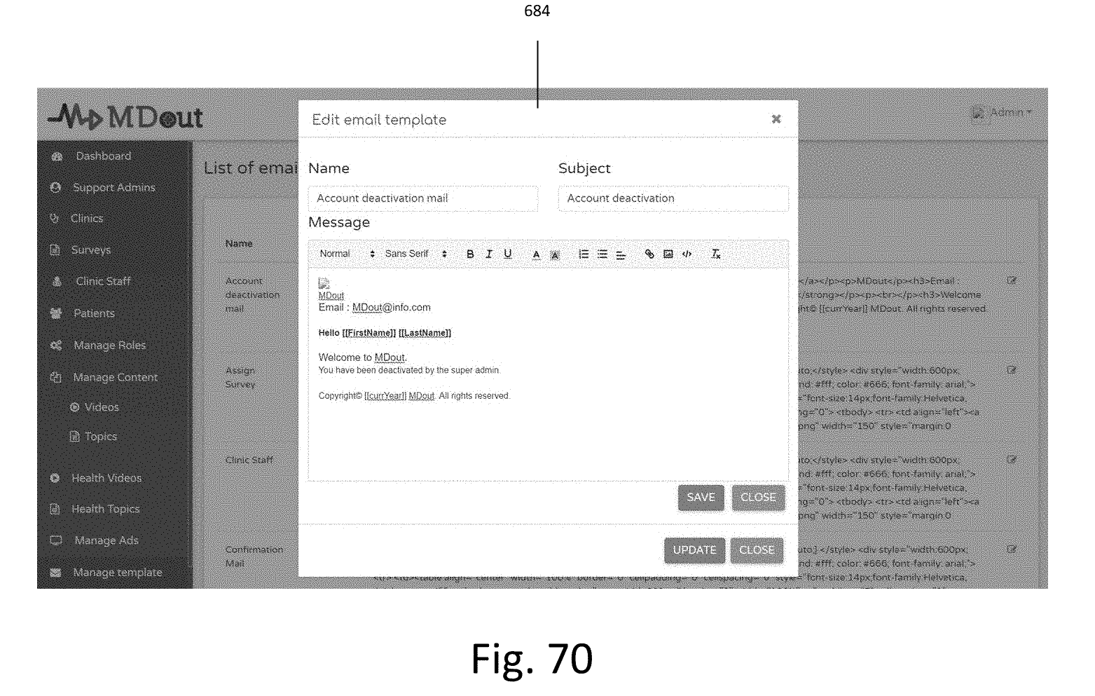

A respective line is assigned to each template. In the list, at least the name, subject, and message of each template is listed. The support administrator also has the ability to edit the templates via the pen and paper icon 682, which will be seen in FIG. 70.

FIG. 70 is a screenshot of a prompt 684 that enables the support administrator to edit a template. Interacting with the pen and paper icon 682 of FIG. 69 causes the server to display the prompt 684. The prompt 684 enables the support administrator to edit the name, subject, and message of the template. At the least, the support administrator has the option to select from various fonts, images, and style choices for the text.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

What is claimed is:

1. A web-based health care information management system comprising at least one computer server connected to a network, the server comprising a processor and a memory unit accessible to the processor, the processor being configured to:
i. generate an interactive website accessible to at least one patient of at least one clinic, at least one staff member of at least one clinic, and at least one administrator of at least one clinic, and at least one system administrator;
ii. provide via the interactive website a system management tool to each system administrator, the processor being configured to:
generate one or more clinic administrator profiles in a first database of clinic administrators, in response to an input from the system administrator;
receive from the system administrator informative videos and articles in a second database of content and receive a tag for each video and article with a category selected from a predetermined list of categories;
receive from the system administrator advertisement videos in a third database of advertisements and receive a tag for each advertisements video with a category selected from the list of categories;
iii. provide via the interactive website a clinic management tool to each clinic administrator, the processor being configured to:
generate one or more profiles of medical staff associated with the clinic to a fourth database of staff members, in response to an input from the clinic administrator;
generate one or more profiles of patients associated with the clinic in a fifth database of patients, in response to an input from the clinic administrator;
receive from the clinic administrator one or more informative videos and articles in the second database and a tag for each video and article with a category selected from the list of categories;
generate questionnaires from scratch or from predetermined questionnaire templates in response to an input from the clinic's administrator, and add the questionnaire to a sixth database of questionnaires;
assign one or more patients to one or more staff members of the clinic in response to an input from the clinic's administrator;
assign one or more of the questionnaires to one or more patients in response to an input from the clinic's administrator;
display to the clinic administrator a screen indicative of a completion status of the assigned questionnaires;
iv. provide via the interactive website a staff management tool to each staff member, the processor being configured to:
display a list of one or more patients assigned to the staff member;
display a list of surveys assigned to the one or more patients assigned to the staff member;
receive from the staff member notes associated with any survey assigned to a patient assigned to the staff member;
display notes previously added to any survey assigned to a patient assigned to the staff member;
v. provide via the interactive web site a patient management tool to each patient, the processor being configured to:
display to the patient a list of surveys assigned to the patient;
receive from the patient answers to one or more of the surveys assigned to the patient;
display one or more informative videos and/or articles from the second database, such that, when the patient views a video or reads an article tagged with a category from the list of categories, at least one advertisement tagged with a category which is the same as the category of the video viewed by the patient or the article read by the user is shown to the user.

2. The system of claim 1, wherein the processor is configured, via the system management tool, to generate one or more of the questionnaire templates and add the templates to the sixth database of questionnaires, in response to an input from the system administrator.

3. The system of claim 1, wherein:
each video and each article added to the system via the clinic management tool is designated as public or private;
a private video is a video viewable by medical staff and patients associated with the clinic;
a public video is a video viewable by all users of the server, pending review by a system administrator;
a private article is an article viewable by medical staff and patients associated with the clinic;
a public article is a video viewable by all users of the server, pending review by a system administrator.

4. The system of claim 1, wherein, when a new private video or a new private article is received in the second database by a clinic's administrator, the server is configured to send a message to each patient associated with the clinic's administrator's clinic to inform each patient that the new private video or the new private article has been added.

5. The system of claim 1, wherein when a questionnaire is assigned to a patient by a clinic administrator, the server is configured to send a message to the patient to inform the patient that the questionnaire has been assigned to the patient.
